(19) United States
(12) Patent Application Publication
(10) Pub. No.: US 2025/0101019 A1
(43) Pub. Date: Mar. 27, 2025

(54) ECTONUCLEOTIDE PYROPHOSPHATASE-PHOSPHODIESTERASE 1 (ENPP1) INHIBITORS AND USES THEREOF

(71) Applicant: Insilico Medicine IP Limited, Hong Kong (CN)

(72) Inventors: Xin CHENG, Shanghai (CN); Yingtao LIU, Shanghai (CN); Luoheng QIN, Shanghai (CN); Feng REN, Shanghai (CN); Huaxing YU, Shanghai (CN)

(21) Appl. No.: 18/832,564

(22) PCT Filed: Jan. 28, 2023
(86) PCT No.: PCT/CN2023/073579

§ 371 Date: Jul. 24, 2024

(30) Foreign Application Priority Data
Jan. 28, 2022 (WO) PCT/CN2022/074739
Dec. 20, 2022 (WO) PCT/CN2022/140318

(51) Int. Cl. C07D 471/04; A61K 31/437; A61K 31/444

(52) U.S. Cl. CPC C07D 471/04; A61K 31/437; A61K 31/444

(57) ABSTRACT

Described herein are ENPP1 inhibitors and pharmaceutical compositions comprising said inhibitors. The subject compounds and compositions are useful for the treatment of a disease or disorder associated with ENPP1.

### CROSS-REFERENCE

This patent application claims the benefit of International Application No. PCT/CN2022/074739, filed Jan. 28, 2022 and International Application No. PCT/CN2022/140318, filed Dec. 20, 2022; which are incorporated herein by reference in their entirety.

### BACKGROUND

Ectonucleotide pyrophosphatase-phosphodiesterase 1 (ENPP1) enzyme is present in a wide range of tissues and cell types, such as lymphocytes, macrophages, liver, brain, heart, kidney, vascular smooth muscle cells, and chondrocytes. ENPP1 hydrolyzes ATP and other nucleoside triphosphates and releases AMP or other nucleoside monophosphates as well as pyrophosphate (PPi). The enzyme can also hydrolyze other nucleoside monophosphate esters. ENPP1 has been identified as the dominant 2′-3′-cGAMP hydrolase in cultured cells, tissue extracts and blood. Tissues and blood from ENPP1 knockout mice lack 2′-3′-cGAMP hydrolase activity. Elevated levels of ENPP1 have been associated with calcific aortic valve disease (CAVD) and calcium pyrophosphate dihydrate (CPPD) disease, an inflammatory disease resulting from CPPD crystal deposits in the joint and surrounding tissues. ENPP1 expression is upregulated in certain hepatocellular carcinomas, glioblastomas, melanomas, testicular, pancreatic, thyroid, and breast cancers and has been associated with resistance to chemotherapy. ENPP1 upregulation and variants of ENPP1 are also associated with insulin resistance and type 2 diabetes and enzyme activity of ENPP1 was reported to be required for the inhibition of insulin receptor signaling.

Cyclic GMP-AMP synthase (cGAS) is a pattern recognition receptor that synthesizes the endogenous messenger molecule cGAMP from ATP and GTP in response to the presence of DNA derived from viruses, bacteria, damaged mitochondria, or cancer cells. The cGAMP molecule then binds to the stimulator of interferon genes (STING) protein, which initiates a signaling response that activates innate immunity and results in the production of type I interferon, antiviral, and immune-stimulatory cytokines. The cGAS enzyme, cGAMP messenger and STING are also involved in host defense against RNA viruses and the immune control of tumor development. ENPP1 has been identified as the enzyme that naturally hydrolyzes cGAMP and therefore counteracts the innate immune response against infectious agents, damaged cells, and cancer cells. The efficacy of non-hydrolyzable cGAMP analogs in inducing functional immune responses is higher than that of natural, hydrolysable cGAMP. Virus infection has been demonstrated to be facilitated by ENPP1 overexpression and is attenuated by silencing of ENPP1.

Inhibitors of cGAMP hydrolysis may therefore be used to increase the effectiveness of immune responses against cancer cells and tumors and against infections by RNA or DNA viruses or bacteria. Inhibitors of ENPP1 and of cGAMP or nucleoside triphosphate hydrolysis may also be used for the treatment of inflammatory diseases that are associated with elevated nucleotidase levels, reduced nucleoside triphosphate, reduced cGAMP or reduced nucleoside monophosphate ester levels or diseases associated with elevated nucleoside or nucleoside monophosphate levels. For these reasons, ENPP1 is an attractive therapeutic target for the treatment of diseases, such as cancers and viral infections.

### SUMMARY

Disclosed herein is a compound of Formula (I), or a pharmaceutically acceptable salt thereof:

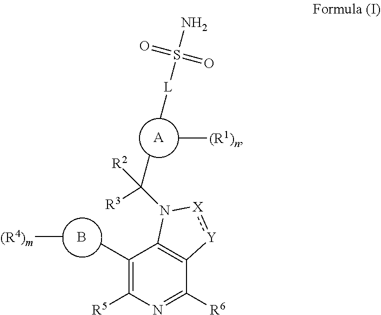

wherein:
Ring A is cycloalkyl, heterocycloalkyl, aryl, or heteroaryl;
L is a bond, —NH—, or —O—;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{c}R^{d}(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R¹ on the same atom are taken together to form an oxo;
n is 0-C₆;
R² is hydrogen, halogen, C₁-C₆ alkyl, C₁-C₆haloalkyl, C₁-C₆ hydroxyalkyl, C₁-C₆aminoalkyl, or C₁-C₆heteroalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
[symbol] is a single bond or a double bond; wherein:
   when [symbol] is a double bond, X is N or CR^{X} and Y is N or CR^{Y};
   when [symbol] is a single bond, X is C(═O) and Y is NR^{Y1} or C(R^{Y2})₂;
R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R^{Y} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R^{Y1} is hydrogen, C₁-C₆alkyl, C₁-C₆haloakyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
each R^{Y2} is independently hydrogen, halogen, —CN, —OH, —OR^{a}, —NR^{c}R^{d}, C₁-C₆ alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R;
Ring B is cycloalkyl, heterocycloalkyl, aryl, or heteroaryl;
each R⁴ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{c}R^{d}(═O) R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R⁴ on the same atom are taken together to form an oxo;
m is 0-4;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆ alkynyl, cycloalkyl, heterocycloalkyl, aryl, or heteroaryl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, or C₁-C₆heteroalkyl; or two R on the same atom form an oxo.

In some embodiments of a compound of Formula (I), the compound is of Formula (Ia):

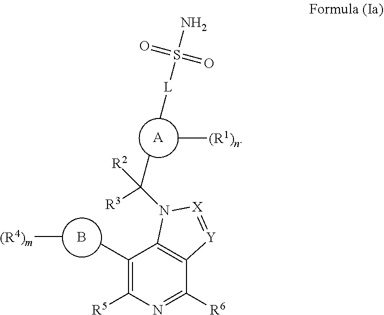

In some embodiments of a compound of Formula (I), the compound is of Formula (Ib):

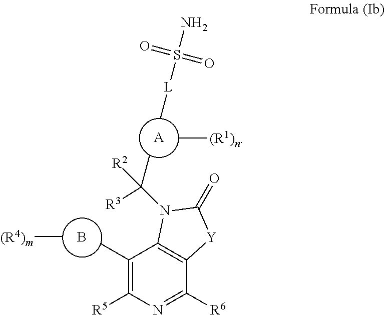

Also disclosed herein is a method of treating cancer in a subject, the method comprising administering to the subject a compound disclosed herein, or a pharmaceutically acceptable salt thereof, or a pharmaceutical composition disclosed herein. In some embodiments, the cancer is a solid tumor. In some embodiments, the solid tumor is breast cancer, lung cancer, ovarian cancer, head and neck cancer, melanoma, pancreatic cancer, liver cancer, gastric cancer, colorectal cancer, or sarcoma. In some embodiments, the cancer is a hematologic malignancy. In some embodiments, the hematologic malignancy is a leukemia, a lymphoma, or a myeloma. In some embodiments, the hematologic malignancy is a B-cell malignancy. In some embodiments, the hematologic malignancy is multiple myeloma. In some embodiments, the cancer is a relapsed or refractory cancer. In some embodiments, the cancer is a metastatic cancer.

Also disclosed herein is a method of treating an infection in a subject in need thereof comprising administering a compound disclosed herein, or a pharmaceutically acceptable salt thereof, or a pharmaceutical composition disclosed herein. In some embodiments, the infection is a viral infection. In some embodiments, the viral infection is due to a DNA virus. In some embodiments, the viral infection is due to a herpesvirus. In some embodiments, the herpesvirus is selected from herpes simplex viruses 1 (HSV-1), herpes simplex viruses 2 (HSV-2), varicella-zoster virus (VZV), Epstein-Barr virus (EBV), human cytomegalovirus (HCMV), human herpesvirus 6A (HHV-6A), human herpesvirus 6B (HHV-6B), human herpesvirus 7 (HHV-7), and Kaposi's sarcoma-associated herpesvirus (KSHV). In some embodiments, the herpesvirus is herpes simplex viruses 1 (HSV-1). In some embodiments, the viral infection is due to a retrovirus. In some embodiments, the retrovirus is human immunodeficiency virus (HIV). In some embodiments, the retrovirus is HIV-1, HIV-2, or human T-lymphotropic virus (HTLV). In some embodiments, the viral infection is due to a hepatitis virus. In some embodiments, the hepatitis virus is hepatitis B virus (HBV) or hepatitis D virus (HDV). In some embodiments, the viral infection is due to vaccinia virus (VACV), adenovirus, or human papillomaviruses (HPV). In some embodiments, the viral infection is due to a RNA virus. In some embodiments, the viral infection is due to dengue fever virus, yellow fever virus, Ebola virus, Marburg virus, Venezuelan encephalitis virus, or zika virus.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:
“oxo” refers to ═O.
“Carboxyl” refers to —COOH.
“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁-C₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁-C₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH—CH₂), 1-propenyl (—CH₂CH—CH₂), isopropenyl [—C(CH₃)—CH₂], butenyl, 1,3-butadienyl and the like. Whenever it appears herein, a numerical range such as “C₂-C₆ alkenyl” or “C₂-C₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂-C₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula-OR^{a} where R^{a} is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl(phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (C₃-C₁₅ fully saturated cycloalkyl or C₃-C₁₅ cycloalkenyl), from three to ten carbon atoms (C₃-C₁₀ fully saturated cycloalkyl or C₃-C₁₀ cycloalkenyl), from three to eight carbon atoms (C₃-C₈ fully saturated cycloalkyl or C₃-C₈ cycloalkenyl), from three to six carbon atoms (C₃-C₆ fully saturated cycloalkyl or C₃-C₆ cycloalkenyl), from three to five carbon atoms (C₃-C₅ fully saturated cycloalkyl or C₃-C₈ cycloalkenyl), or three to four carbon atoms (C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃) OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ fully saturated heterocycloalkyl or C₂-C₁₅ heterocycloalkenyl), from two to ten carbon atoms (C₂-C₁₀ fully saturated heterocycloalkyl or C₂-C₁₀ heterocycloalkenyl), from two to eight carbon atoms (C₂-C₈ fully saturated heterocycloalkyl or C₂-C₈ heterocycloalkenyl), from two to seven carbon atoms (C₂-C₇ fully saturated heterocycloalkyl or C₂-C₇ heterocycloalkenyl), from two to six carbon atoms (C₂-C₆ fully saturated heterocycloalkyl or C₂-C₆ heterocycloalkenyl), from two to five carbon atoms (C₂-C₅ fully saturated heterocycloalkyl or C₂-C₅ heterocycloalkenyl), or two to four carbon atoms (C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl [1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl(benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, four substituents, or more substituents. In some embodiments, the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

As used herein, a “disease or disorder associated with ENPP1” or, alternatively, “a ENPP1-mediated disease or disorder” means any disease or other deleterious condition in which ENPP1, or a mutant thereof, is known or suspected to play a role.

### Compounds

Described herein are compounds, or a pharmaceutically acceptable salt thereof useful in the treatment of a disease or disorder associated with ENPP1.

Disclosed herein is a compound of Formula (A), or a pharmaceutically acceptable salt thereof:

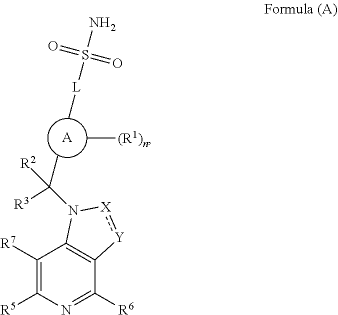

wherein:
Ring A is cycloalkyl, heterocycloalkyl, aryl, or heteroaryl;
L is a bond, —NH—, or —O—;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{c}R^{d}(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R¹ on the same atom are taken together to form an oxo;
n is 0-C₆;
R² is hydrogen, halogen, C₁-C₆alkyl, C₁-C₆haloalkyl, C₁-C₆ hydroxyalkyl, C₁-C₆aminoalkyl, or C₁-C₆heteroalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
[symbol] is a single bond or a double bond; wherein:
   when [symbol] is a double bond, X is N or CR^{X} and Y is N or CR^{Y};
   when [symbol] is a single bond, X is C(═O) and Y is NR^{Y1} or C(R^{Y2})₂;
R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R^{Y} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R^{Y1} is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
each R^{Y2} is independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R;
R⁷ is hydrogen, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is substituted with one or more R⁸;
each R⁸ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{c}R^{d}(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R⁸ on the same atom are taken together to form an oxo;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d},
C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆ alkynyl, cycloalkyl, heterocycloalkyl, aryl, or heteroaryl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆ alkynyl, cycloalkyl, heterocycloalkyl, aryl, or heteroaryl;
each R^{a} is independently C₁-C₆alkyl, C₁-C₆haloalkyl, C₁-C₆hydroxyalkyl, C₁-C₆aminoalkyl,
C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl,
C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆ hydroxyalkyl, C₁-C₆aminoalkyl, C₁-C₆heteroalkyl, cycloalkyl, or heterocycloalkyl;
or two R on the same atom form an oxo.

In some embodiments of a compound of Formula (A), R⁷ is C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is substituted with one or more R⁸.

In some embodiments of a compound of Formula (A), R⁷ is C₁-C₆alkyl, C₁-C₆haloalkyl, C₂-C₆ alkynyl, cycloalkyl, heterocycloalkyl, aryl, or heteroaryl; wherein the alkyl, alkynyl, cycloalkyl, heterocycloalkyl, aryl, and heteroaryl is substituted with one or more R⁸.

In some embodiments of a compound of Formula (A), R⁷ is C₁-C₆alkyl, C₂-C₆alkynyl, cycloalkyl, heterocycloalkyl, aryl, or heteroaryl; wherein the alkyl, alkynyl, cycloalkyl, heterocycloalkyl, aryl, and heteroaryl is substituted with one or more R⁸.

In some embodiments of a compound of Formula (A), R⁷ is C₁-C₆alkyl or C₂-C₆alkynyl; wherein the alkyl and alkynyl is substituted with one or more R⁸.

In some embodiments of a compound of Formula (A), R⁷ is cycloalkyl, heterocycloalkyl, aryl, or heteroaryl; wherein the cycloalkyl, heterocycloalkyl, aryl, and heteroaryl is substituted with one or more R⁸.

In some embodiments of a compound of Formula (A), R⁷ is aryl or heteroaryl; wherein the aryl and heteroaryl is substituted with one or more R⁸.

In some embodiments of a compound of Formula (A), R⁷ is aryl substituted with one or more R⁸.

Disclosed herein is a compound of Formula (I), or a pharmaceutically acceptable salt thereof:

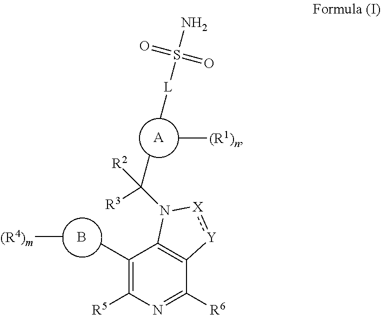

wherein:
Ring A is cycloalkyl, heterocycloalkyl, aryl, or heteroaryl;
L is a bond, —NH—, or —O—;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R¹ on the same atom are taken together to form an oxo;
n is 0-C₆;
R² is hydrogen, halogen, C₁-C₆alkyl, C₁-C₆haloalkyl, C₁-C₆hydroxyalkyl, C₁-C₆aminoalkyl, or C₁-C₆heteroalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
[symbol] is a single bond or a double bond; wherein:
   when [symbol] is a double bond, X is N or CR^{X} and Y is N or CR^{Y};
   when [symbol] is a single bond, X is C(═O) and Y is NR^{Y1} or C(R^{Y2})₂;
R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R^{Y} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R^{Y1} is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
each R^{Y2} is independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R;
Ring B is cycloalkyl, heterocycloalkyl, aryl, or heteroaryl;
each R⁴ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{c}R^{d}(═O) R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R⁴ on the same atom are taken together to form an oxo;
m is 0-4;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆ alkyl, C₁-C₆haloalkyl, C₁-C₆hydroxyalkyl, C₁-C₆aminoalkyl, C₁-C₆heteroalkyl, C₂-C₆alkenyl, C₂-C₆ alkynyl, cycloalkyl, heterocycloalkyl, aryl, or heteroaryl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆ alkyl, C₁-C₆haloalkyl, C₁-C₆hydroxyalkyl, C₁-C₆aminoalkyl, C₁-C₆heteroalkyl, cycloalkyl, or heterocycloalkyl;
or two R on the same atom form an oxo.

Disclosed herein is a compound of Formula (I), or a pharmaceutically acceptable salt thereof:

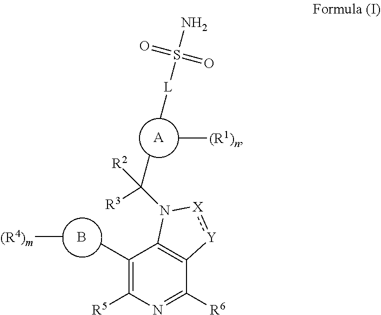

wherein:
Ring A is cycloalkyl, heterocycloalkyl, aryl, or heteroaryl;
L is a bond, —NH—, or —O—;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆ hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R¹ on the same atom are taken together to form an oxo;
n is 0-C₆;
R² is hydrogen, halogen, C₁-C₆alkyl, C₁-C₆haloalkyl, C₁-C₆ hydroxyalkyl, C₁-C₆aminoalkyl, or C₁-C₆heteroalkyl;
R³ is hydrogen, halogen, C₁-C₆alkyl, C₁-C₆haloalkyl, C₁-C₆hydroxyalkyl, C₁-C₆aminoalkyl, or C₁-C₆heteroalkyl;
[symbol] is a single bond or a double bond; wherein:
   when [symbol] is a double bond, X is N or CR^{X} and Y is N or CR^{Y};
   when [symbol] is a single bond, X is C(═O) and Y is NR^{Y1} or C(R^{Y2})₂;
R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R^{Y} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R^{Y1} is hydrogen, C₁-C₆ alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
each R^{Y2} is independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R;
Ring B is cycloalkyl, heterocycloalkyl, aryl, or heteroaryl;
each R⁴ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R⁴ on the same atom are taken together to form an oxo;
m is 0-4;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆ alkynyl, cycloalkyl, heterocycloalkyl, aryl, or heteroaryl;
each R^{a} is independently C₁-C₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, or C₁-C₆heteroalkyl;
or two R on the same atom form an oxo.

In some embodiments of a compound of Formula (I), the compound is of Formula (Ia):

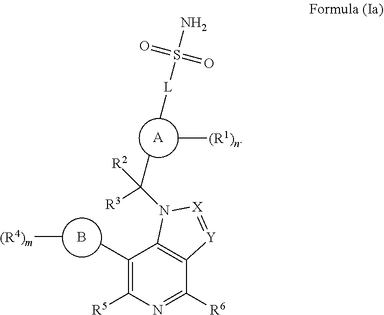

In some embodiments of a compound of Formula (A), (I), or (Ia), X is N and Y is N. In some embodiments of a compound of Formula (A), (I), or (Ia), X is CR^{X} and Y is N. In some embodiments of a compound of Formula (A), (I), or (Ia), X is C(C₁-C₃alkyl) and Y is N. In some embodiments of a compound of Formula (A), (I), or (Ia), X is N and Y is CR^{Y}. In some embodiments of a compound of Formula (A), (I), or (Ia), X is CR^{X} and Y is CR^{Y}.

In some embodiments of a compound of Formula (A), (I), or (Ia), R^{X} is hydrogen, halogen, C₁-C₆alkyl, or C₁-C₆haloalkyl. In some embodiments of a compound of Formula (A), (I), or (Ia), R^{X} is hydrogen, halogen, or C₁-C₆alkyl. In some embodiments of a compound of Formula (A), (I), or (Ia), R^{X} is hydrogen, halogen, C₁-C₆alkyl, C₁-C₆haloalkyl, or cycloalkyl. In some embodiments of a compound of Formula (A), (I), or (Ia), R^{X} is hydrogen, C₁-C₆ alkyl, C₁-C₆haloalkyl, or cycloalkyl. In some embodiments of a compound of Formula (A), (I), or (Ia), R^{X} is methyl. In some embodiments of a compound of Formula (A), (I), or (Ia), R^{X} is CD₃. In some embodiments of a compound of Formula (A), (I), or (Ia), R^{X} is cyclopropyl. In some embodiments of a compound of Formula (A), (I), or (Ia), R^{X} is halogen.

In some embodiments of a compound of Formula (A), (I), or (Ia), R^{Y} is hydrogen, halogen, C₁-C₆alkyl, C₁-C₆haloalkyl, or cycloalkyl. In some embodiments of a compound of Formula (A), (I), or (Ia), R^{Y} is hydrogen, halogen, C₁-C₆alkyl, or C₁-C₆haloalkyl. In some embodiments of a compound of Formula (A), (I), or (Ia), R^{Y} is hydrogen or C₁-C₆alkyl. n some embodiments of a compound of Formula (A), (I), or (Ia), R^{Y} is hydrogen. n some embodiments of a compound of Formula (A), (I), or (Ia), R^{Y} is C₁-C₆alkyl.

In some embodiments of a compound of Formula (I), the compound is of Formula (Ib):

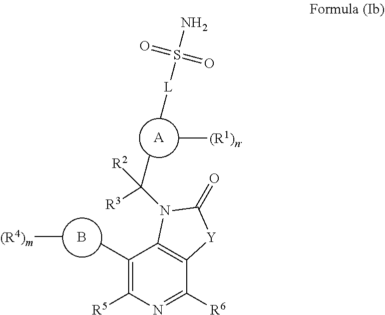

In some embodiments of a compound of Formula (A), (I), or (Ib), Y is NR^{Y1}. In some embodiments of a compound of Formula (A), (I), or (Ib), Y is C(R^{Y2})₂.

In some embodiments of a compound of Formula (A), (I), or (Ib), R^{Y1} is hydrogen, C₁-C₆alkyl, or C₁-C₆haloalkyl. In some embodiments of a compound of Formula (A), (I), or (Ib), R^{Y1} is C₁-C₆alkyl or C₁-C₆haloalkyl. In some embodiments of a compound of Formula (A), (I), or (Ib), R^{Y1} is C₁-C₃alkyl or C₁-C₃haloalkyl.

In some embodiments of a compound of Formula (A), (I), or (Ib), each R^{Y2} is independently hydrogen, halogen, C₁-C₆alkyl, or C₁-C₆haloalkyl. In some embodiments of a compound of Formula (A), (I), or (Ib), each R^{Y2} is independently hydrogen or C₁-C₆alkyl.

In some embodiments of a compound of Formula (A), (I), (Ia), or (Ib), Ring A is cycloalkyl or heterocycloalkyl.

In some embodiments of a compound of Formula (A), (I), (Ia), or (Ib), Ring A is cycloalkyl. In some embodiments of a compound of Formula (A), (I), (Ia), or (Ib), Ring A is 3- to 12-membered cycloalkyl. In some embodiments of a compound of Formula (A), (I), (Ia), or (Ib), Ring A is 5- or 6-membered cycloalkyl. In some embodiments of a compound of Formula (A), (I), (Ia), or (Ib), Ring A is 3- to 7-membered monocyclic cycloalkyl. In some embodiments of a compound of Formula (A), (I), (Ia), or (Ib), Ring A is 5- to 12-membered bicyclic cycloalkyl. In some embodiments of a compound of Formula (A), (I), (Ia), or (Ib), Ring A is 7- to 12-membered bicyclic cycloalkyl. In some embodiments of a compound of Formula (A), (I), (Ia), or (Ib), Ring A is 9- to 12-membered bicyclic cycloalkyl.

In some embodiments of a compound of Formula (A), (I), (Ia), or (Ib), Ring A is heterocycloalkyl. In some embodiments of a compound of Formula (A), (I), (Ia), or (Ib), Ring A is 3- to 12-membered heterocycloalkyl comprising 1 to 3 heteroatoms selected from N, O, or S. In some embodiments of a compound of Formula (A), (I), (Ia), or (Ib), Ring A is 5- or 6-membered heterocycloalkyl. In some embodiments of a compound of Formula (A), (I), (Ia), or (Ib), Ring A is 3- to 7-membered monocyclic heterocycloalkyl comprising 1 to 3 heteroatoms selected from N, O, or S. In some embodiments of a compound of Formula (A), (I), (Ia), or (Ib), Ring A is 5- to 12-membered bicyclic heterocycloalkyl comprising 1 to 3 heteroatoms selected from N, O, or S. In some embodiments of a compound of Formula (A), (I), (Ia), or (Ib), Ring A is 7- to 12-membered bicyclic heterocycloalkyl comprising 1 to 3 heteroatoms selected from N, O, or S. In some embodiments of a compound of Formula (A), (I), (Ia), or (Ib), Ring A is 9- to 12-membered bicyclic heterocycloalkyl comprising 1 to 3 heteroatoms selected from N, O, or S.

In some embodiments of a compound of Formula (A), (I), (Ia), or (Ib), Ring A is aryl or heteroaryl. In some embodiments of a compound of Formula (A), (I), (Ia), or (Ib), Ring A is aryl. In some embodiments of a compound of Formula (A), (I), (Ia), or (Ib), Ring A is phenyl. In some embodiments of a compound of Formula (A), (I), (Ia), or (Ib), Ring A is heteroaryl. In some embodiments of a compound of Formula (A), (I), (Ia), or (Ib), Ring A is 6 membered monocyclic heteroaryl. In some embodiments of a compound of Formula (A), (I), (Ia), or (Ib), Ring A is pyridinyl. In some embodiments of a compound of Formula (A), (I), (Ia), or (Ib), Ring A is pyridazinyl. In some embodiments of a compound of Formula (A), (I), (Ia), or (Ib), Ring A is pyrimidinyl. In some embodiments of a compound of Formula (A), (I), (Ia), or (Ib), Ring A is pyrazinyl.

In some embodiments of a compound of Formula (A), (I), (Ia), or (Ib), Ring A is a spiro bicyclic ring. In some embodiments of a compound of Formula (A), (I), (Ia), or (Ib), Ring A is a fused bicyclic ring.

In some embodiments of a compound of Formula (A), (I), (Ia), or (Ib), L is a bond. In some embodiments of a compound of Formula (A), (I), (Ia), or (Ib), L is —NH—. In some embodiments of a compound of Formula (A), (I), (Ia), or (Ib), L is —O—.

In some embodiments of a compound of Formula (A), (I), (Ia), or (Ib), each R¹ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl. In some embodiments of a compound of Formula (A), (I), (Ia), or (Ib), each R¹ is independently halogen, —CN, —OH, —OR^{a}, —NR^{c}R^{d}, C₁-C₆alkyl, or C₁-C₆haloalkyl. In some embodiments of a compound of Formula (A), (I), (Ia), or (Ib), each R¹ is independently halogen, C₁-C₆alkyl, or C₁-C₆haloalkyl. In some embodiments of a compound of Formula (A), (I), (Ia), or (Ib), each R¹ is independently halogen. In some embodiments of a compound of Formula (A), (I), (Ia), or (Ib), R¹ is F.

In some embodiments of a compound of Formula (A), (I), (Ia), or (Ib), n is 0-2. In some embodiments of a compound of Formula (A), (I), (Ia), or (Ib), n is 0 or 1. In some embodiments of a compound of Formula (A), (I), (Ia), or (Ib), n is 1 or 2. In some embodiments of a compound of Formula (A), (I), (Ia), or (Ib), n is 0. In some embodiments of a compound of Formula (A), (I), (Ia), or (Ib), n is 1. In some embodiments of a compound of Formula (A), (I), (Ia), or (Ib), n is 2. In some embodiments of a compound of Formula (A), (I), (Ia), or (Ib), n is 3.

In some embodiments of a compound of Formula (A), (I), (Ia), or (Ib),

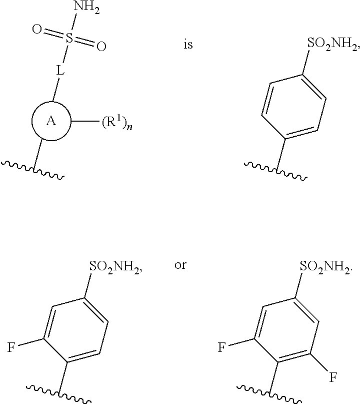

In some embodiments of a compound of Formula (A), (I), (Ia), or (Ib),

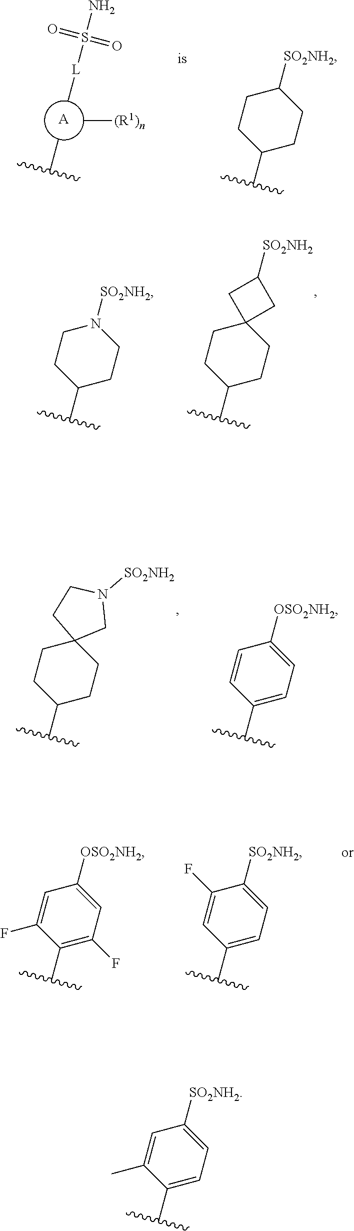

In some embodiments of a compound of Formula (A), (I), (Ia), or (Ib),

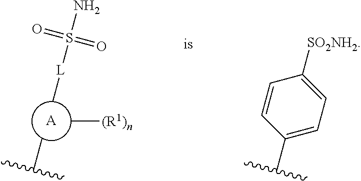

In some embodiments of a compound of Formula (A), (I), (Ia), or (Ib),

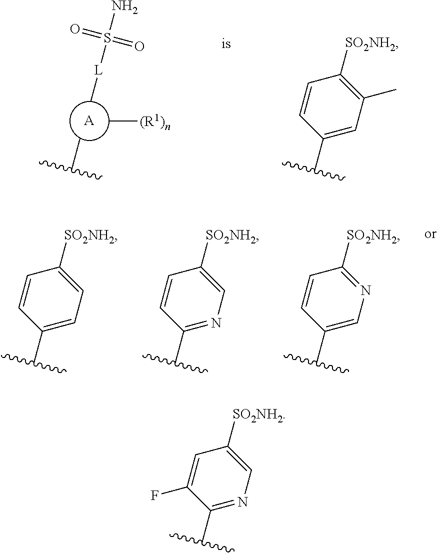

In some embodiments of a compound of Formula (A), (I), (Ia), or (Ib),

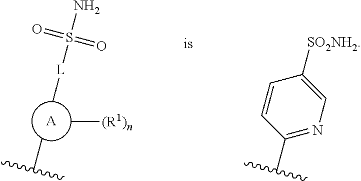

In some embodiments of a compound of Formula (A), (I), (Ia), or (Ib),

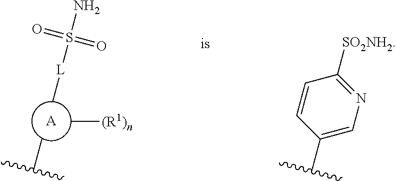

In some embodiments of a compound of Formula (A), (I), (Ia), or (Ib),

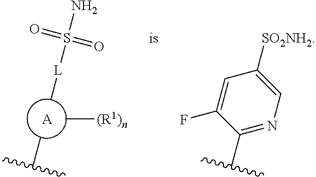

In some embodiments of a compound of Formula (A), (I), (Ia), or (Ib),

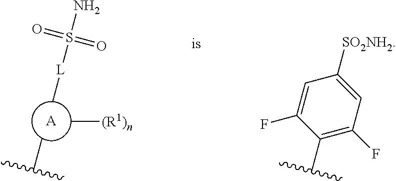

In some embodiments of a compound of Formula (A), (I), (Ia), or (Ib), R² is hydrogen, halogen, C₁-C₆ alkyl, or C₁-C₆haloalkyl. In some embodiments of a compound of Formula (A), (I), (Ia), or (Ib), R² is hydrogen or C₁-C₆alkyl. In some embodiments of a compound of Formula (A), (I), (Ia), or (Ib), R² is hydrogen. In some embodiments of a compound of Formula (A), (I), (Ia), or (Ib), R² is C₁-C₆alkyl.

In some embodiments of a compound of Formula (A), (I), (Ia), or (Ib), R³ is hydrogen, halogen, C₁-C₆alkyl, or C₁-C₆haloalkyl. In some embodiments of a compound of Formula (A), (I), (Ia), or (Ib), R³ is hydrogen or C₁-C₆alkyl. In some embodiments of a compound of Formula (A), (I), (Ia), or (Ib), R³ is hydrogen. In some embodiments of a compound of Formula (A), (I), (Ia), or (Ib), R³ is C₁-C₆alkyl.

In some embodiments of a compound of Formula (I), (Ia), or (Ib), Ring B is cycloalkyl or heterocycloalkyl. In some embodiments of a compound of Formula (I), (Ia), or (Ib), Ring B is cycloalkyl. In some embodiments of a compound of Formula (I), (Ia), or (Ib), Ring B is 5- or 6-membered cycloalkyl. In some embodiments of a compound of Formula (I), (Ia), or (Ib), Ring B is heterocycloalkyl. In some embodiments of a compound of Formula (I), (Ia), or (Ib), Ring B is 5- or 6-membered heterocycloalkyl. In some embodiments of a compound of Formula (I), (Ia), or (Ib), Ring B is a fully saturated ring. In some embodiments of a compound of Formula (I), (Ia), or (Ib), Ring B is partially saturated ring.

In some embodiments of a compound of Formula (I), (Ia), or (Ib), Ring B is aryl or heteroaryl. In some embodiments of a compound of Formula (I), (Ia), or (Ib), Ring B is aryl. In some embodiments of a compound of Formula (I), (Ia), or (Ib), Ring B is phenyl. In some embodiments of a compound of Formula (I), (Ia), or (Ib), Ring B is heteroaryl. In some embodiments of a compound of Formula (I), (Ia), or (Ib), Ring B is 5- or 6-membered heteroaryl. Ring B is 5-membered heteroaryl. Ring B is 6-membered heteroaryl. In some embodiments of a compound of Formula (I), (Ia), or (Ib), Ring B is heteroaryl containing 1-3 nitrogen and 0-1 oxygen. In some embodiments of a compound of Formula (I), (Ia), or (Ib), Ring B is heteroaryl containing 2-4 nitrogens. In some embodiments of a compound of Formula (I), (Ia), or (Ib), Ring B is heteroaryl containing 1 or 2 nitrogen.

In some embodiments of a compound of Formula (I), (Ia), or (Ib), each R⁴ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl. In some embodiments of a compound of Formula (I), (Ia), or (Ib), each R⁴ is independently halogen, —CN, —OH, —OR^{a}, —NR^{c}R^{d}, C₁-C₆alkyl, or C₁-C₆haloalkyl. In some embodiments of a compound of Formula (I), (Ia), or (Ib), each R⁴ is independently halogen, C₁-C₆alkyl, or C₁-C₆haloalkyl. In some embodiments of a compound of Formula (I), (Ia), or (Ib), each R⁴ is independently C₁-C₆alkyl.

In some embodiments of a compound of Formula (I), (Ia), or (Ib), wherein m is 0-2. In some embodiments of a compound of Formula (I), (Ia), or (Ib), m is 0 or 1. In some embodiments of a compound of Formula (I), (Ia), or (Ib), m is 0. In some embodiments of a compound of Formula (I), (Ia), or (Ib), m is 1. In some embodiments of a compound of Formula (I), (Ia), or (Ib), m is 2. In some embodiments of a compound of Formula (I), (Ia), or (Ib), m is 3.

In some embodiments of a compound of Formula (I), (Ia), or (Ib),

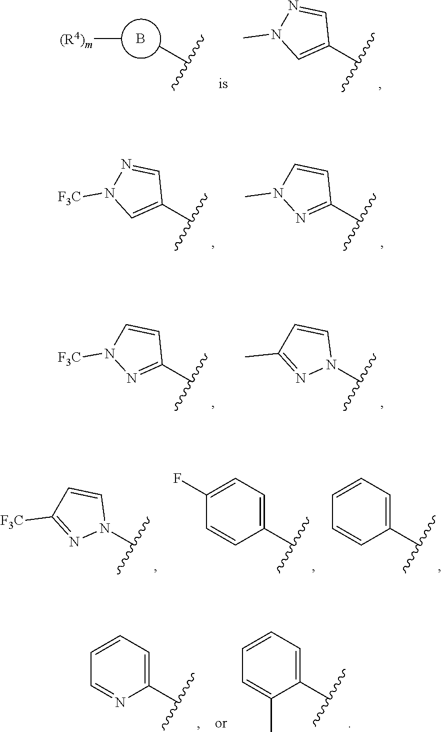

In some embodiments of a compound of Formula (I), (Ia), or (Ib),

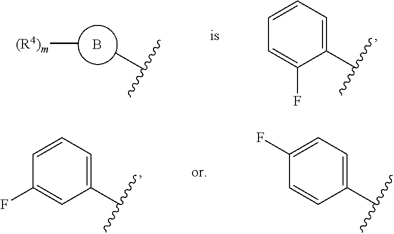

In some embodiments of a compound of Formula (I), (Ia), or (Ib),

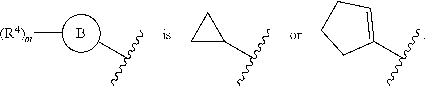

In some embodiments of a compound of Formula (I), (Ia), or (Ib),

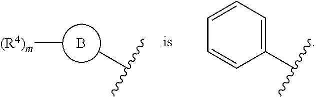

In some embodiments of a compound of Formula (A), (I), (Ia), or (Ib), R⁵ is hydrogen, halogen, —CN, —OR^{a}, —NR^{c}R^{d}, C₁-C₆alkyl, or C₁-C₆haloalkyl. In some embodiments of a compound of Formula (A), (I), (Ia), or (Ib), R⁵ is hydrogen, —CN, —OR^{a}, C₁-C₆alkyl, or C₁-C₆haloalkyl. In some embodiments of a compound of Formula (A), (I), (Ia), or (Ib), R⁵ is hydrogen, —CN, —OR^{a}, or C₁-C₆alkyl. In some embodiments of a compound of Formula (A), (I), (Ia), or (Ib), R⁵ is hydrogen, halogen, —OR^{a}, or C₁-C₆alkyl. In some embodiments of a compound of Formula (A), (I), (Ia), or (Ib), R⁵ is —OR^{a} or C₁-C₆alkyl. In some embodiments of a compound of Formula (A), (I), (Ia), or (Ib), R⁵ is C₁-C₆alkoxyl or C₁-C₆alkyl. In some embodiments of a compound of Formula (A), (I), (Ia), or (Ib), R⁵ is C₁-C₃alkoxyl or C₁-C₃alkyl. In some embodiments of a compound of Formula (A), (I), (Ia), or (Ib), R⁵ is —OR^{a}. In some embodiments of a compound of Formula (A), (I), (Ia), or (Ib), R⁵ is C₁-C₆alkoxyl. In some embodiments of a compound of Formula (A), (I), (Ia), or (Ib), R⁵ is OCH₃. In some embodiments, the OCH₃ is OCD₃. In some embodiments of a compound of Formula (A), (I), (Ia), or (Ib), R⁵ is C₁-C₃alkyl. In some embodiments of a compound of Formula (A), (I), (Ia), or (Ib), R⁵ is methyl. In some embodiments of a compound of Formula (A), (I), (Ia), or (Ib), R⁵ is —CN. In some embodiments of a compound of Formula (A), (I), (Ia), or (Ib), R⁵ is —O—C₁-C₃alkyl. In some embodiments of a compound of Formula (A), (I), (Ia), or (Ib), R⁵ is —OMe.

In some embodiments of a compound of Formula (A), (I), (Ia), or (Ib), R⁶ is hydrogen, halogen, —CN, —OH, —OR^{a}, —NR^{c}R^{d}, C₁-C₆alkyl, or C₁-C₆haloalkyl. In some embodiments of a compound of Formula (A), (I), (Ia), or (Ib), R⁶ is hydrogen, halogen, —CN, C₁-C₆alkyl, or C₁-C₆haloalkyl. In some embodiments of a compound of Formula (A), (I), (Ia), or (Ib), R⁶ is hydrogen, —CN, or C₁-C₆alkyl. In some embodiments of a compound of Formula (A), (I), (Ia), or (Ib), R⁶ is hydrogen or C₁-C₆alkyl. In some embodiments of a compound of Formula (A), (I), (Ia), or (Ib), R⁶ is hydrogen.

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkyl(cycloalkyl), C₁-C₆alkylene (heterocycloalkyl), C₁-C₆alkyl(aryl), or C₁-C₆alkyl(heteroaryl); wherein each alkyl, cycloalkyl, heterocycloalkyl, aryl, and heteroaryl is independently optionally substituted with one or more R. In some embodiments of a compound disclosed herein, each R^{a} is independently C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene (cycloalkyl), C₁-C₆alkylene (heterocycloalkyl), C₁-C₆alkylene (aryl), or C₁-C₆alkylene (heteroaryl). In some embodiments of a compound disclosed herein, each R^{a} is independently C₁-C₆alkyl, C₁-C₆haloalkyl, or cycloalkyl, heterocycloalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene (cycloalkyl), C₁-C₆alkylene (heterocycloalkyl), C₁-C₆alkylene (aryl), or C₁-C₆alkylene (heteroaryl); wherein each alkyl, cycloalkyl, heterocycloalkyl, aryl, and heteroaryl is independently optionally substituted with one or more R. In some embodiments of a compound disclosed herein, each R^{b} is independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{b} is independently hydrogen, C₁-C₆ alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene (cycloalkyl), C₁-C₆alkylene (heterocycloalkyl), C₁-C₆alkylene (aryl), or C₁-C₆alkylene (heteroaryl). In some embodiments of a compound disclosed herein, each R^{b} is independently hydrogen, C₁-C₆alkyl, C₁-C₆haloalkyl, or cycloalkyl, heterocycloalkyl. In some embodiments of a compound disclosed herein, each R^{b} is independently hydrogen, C₁-C₆alkyl or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆ hydroxyalkyl, C₁-C₆aminoalkyl, C₁-C₆heteroalkyl, cycloalkyl, heterocycloalkyl, aryl, heteroaryl, C₁-C₆alkylene (cycloalkyl), C₁-C₆alkylene (heterocycloalkyl), C₁-C₆alkylene (aryl), or C₁-C₆alkylene (heteroaryl); wherein each alkyl, cycloalkyl, heterocycloalkyl, aryl, and heteroaryl is independently optionally substituted with one or more R. In some embodiments of a compound disclosed herein, each R^{c} and R^{d} are independently hydrogen, C₁-C₆alkyl, C₁-C₆haloalkyl, or cycloalkyl, heterocycloalkyl; wherein each alkyl, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene (cycloalkyl), C₁-C₆alkylene (heterocycloalkyl), C₁-C₆alkylene (aryl), or C₁-C₆alkyl(heteroaryl). In some embodiments of a compound disclosed herein, each R^{c} and R^{d} are independently hydrogen, C₁-C₆alkyl, C₁-C₆haloalkyl, or cycloalkyl, heterocycloalkyl. In some embodiments of a compound disclosed herein, each R^{c} and R^{d} are independently hydrogen, C₁-C₆alkyl or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —OC₁-C₆alkyl, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, cycloalkyl or heterocycloalkyl. In some embodiments of a compound disclosed herein, each R is independently halogen, —CN, —OH, —OC₁-C₆alkyl, —NH₂, —NHC₁-C₆alkyl, —N(C₁-C₆alkyl)₂, —NHC(═O)OC₁-C₆alkyl, —C(═O)C₁-C₆alkyl, —C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, or C₁-C₆haloalkyl. In some embodiments of a compound disclosed herein, each R is independently halogen, —CN, —OH, —OC₁-C₆alkyl, —NH₂, —C(═O)C₁-C₆alkyl, —C(═O)OH, —C(═O)OC₁-C₆alkyl, —C(═O)NH₂, C₁-C₆alkyl, or C₁-C₆haloalkyl. In some embodiments of a compound disclosed herein, each R is independently halogen, —CN, —OH, —OC₁-C₆alkyl, —NH₂, C₁-C₆ alkyl, or C₁-C₆haloalkyl.

In some embodiments of a compound disclosed herein, one or more of R, R¹, R², R³, R⁴, R⁵, R⁶, R⁷, R⁸, R^{X}, R^{Y}, R^{Y1}, R^{Y2}, R^{a}, R^{b}, R^{c}, and R^{d} groups comprise deuterium at a percentage higher than the natural abundance of deuterium.

In some embodiments of a compound disclosed herein, one or more ¹H are replaced with one or more deuteriums in one or more of the following groups R, R¹, R², R³, R⁴, R⁵, R⁶, R⁷, R⁸, R^{X}, R^{Y}, R^{Y1}, R^{Y2}, R^{a}, R^{b}, R^{c}, and R^{d}.

In some embodiments of a compound disclosed herein, the abundance of deuterium in each of R, R¹, R², R³, R⁴, R⁵, R⁶, R⁷, R⁸, R^{X}, R^{Y}, R^{Y1}, R^{Y2}, R^{a}, R^{b}, Re, and R^{d} is independently at least 1%, at least 10%, at least 20%, at least 30%, at least 40%, at least 50%, at least 60%, at least 70%, at least 80%, at least 90%, or 100% by molar.

In some embodiments of a compound disclosed herein, one or more 1H of Ring A or Ring B are replaced with one or more deuteriums.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt thereof, is one of the compounds in Table 1.

TABLE 1

| Example | Structure |
|---|---|
| 1 | 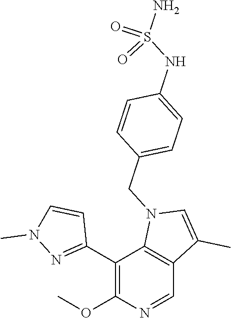 |
| 2 | 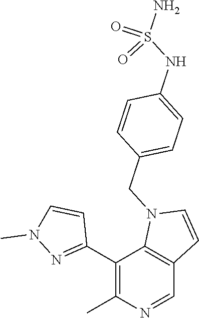 [SMILES: Cc1ncc2ccn(Cc3ccc(NS(N)(=O)=O)cc3)c2c1-c1ccn(C)n1] |
| 3 | 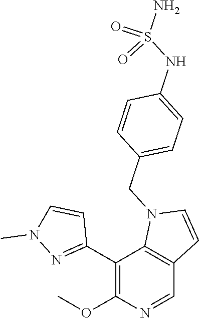 [SMILES: COc1ncc2ccn(Cc3ccc(NS(N)(=O)=O)cc3)c2c1-c1ccn(C)n1] |
| 4 | 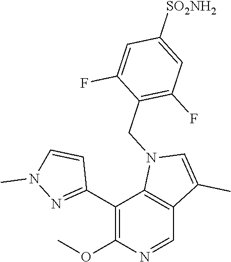 |
| 5 | 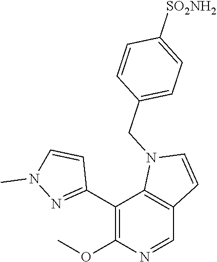 [SMILES: COc1ncc2ccn(Cc3ccc(S(N)(=O)=O)cc3)c2c1-c1ccn(C)n1] |
| 6 | 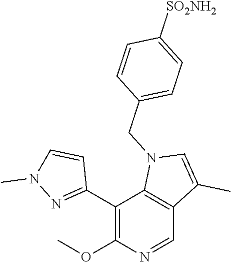 |
| 7 | 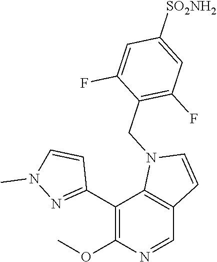 [SMILES: COc1ncc2ccn(Cc3c(F)cc(S(N)(=O)=O)cc3F)c2c1-c1ccn(C)n1] |
| 8 | 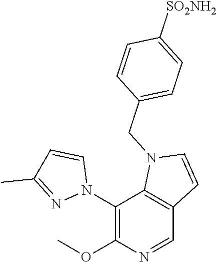 [SMILES: COc1ncc2ccn(Cc3ccc(S(N)(=O)=O)cc3)c2c1-n1ccc(C)n1] |
| 9 | 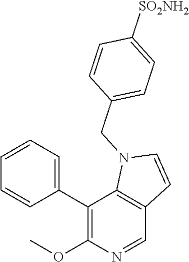 [SMILES: COc1ncc2ccn(Cc3ccc(S(N)(=O)=O)cc3)c2c1-c1ccccc1] |
| 10 | 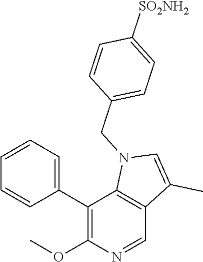 [SMILES: COc1ncc2c(C)cn(Cc3ccc(S(N)(=O)=O)cc3)c2c1-c1ccccc1] |
| 11 | 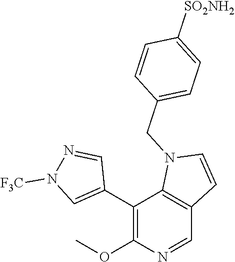 [SMILES: COc1ncc2ccn(Cc3ccc(S(N)(=O)=O)cc3)c2c1-c1cnn(C(F)(F)F)c1] |
| 12 | 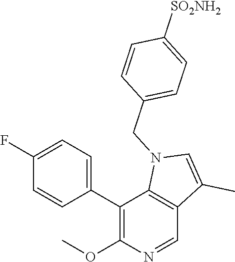 [SMILES: COc1ncc2c(C)cn(Cc3ccc(S(N)(=O)=O)cc3)c2c1-c1ccc(F)cc1] |
| 13 | 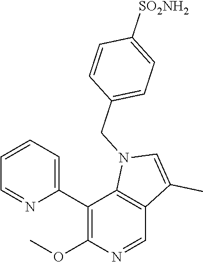 [SMILES: COc1ncc2c(C)cn(Cc3ccc(S(N)(=O)=O)cc3)c2c1-c1ccccn1] |
| 14 | 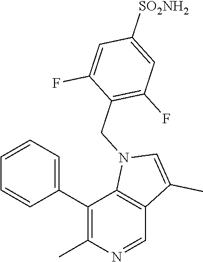 [SMILES: Cc1ncc2c(C)cn(Cc3c(F)cc(S(N)(=O)=O)cc3F)c2c1-c1ccccc1] |
| 15 | 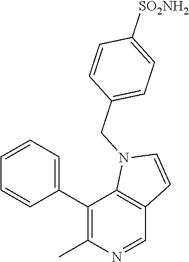 [SMILES: Cc1ncc2ccn(Cc3ccc(S(N)(=O)=O)cc3)c2c1-c1ccccc1] |
| 16 | 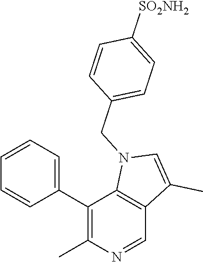 [SMILES: Cc1ncc2c(C)cn(Cc3ccc(S(N)(=O)=O)cc3)c2c1-c1ccccc1] |
| 17 | 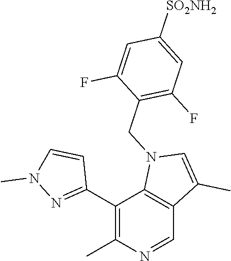 |
| 18 | 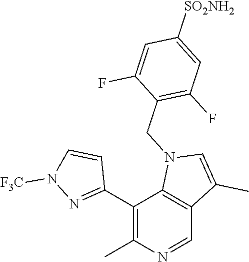 [SMILES: Cc1ncc2c(C)cn(Cc3c(F)cc(S(N)(=O)=O)cc3F)c2c1-c1ccn(C(F)(F)F)n1] |
| 19 | 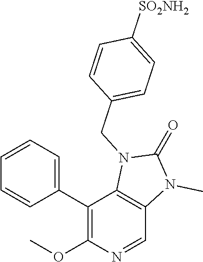 [SMILES: COc1ncc2c(c1-c1ccccc1)n(Cc1ccc(S(N)(=O)=O)cc1)c(=O)n2C] |
| 20 | 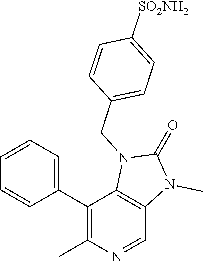 [SMILES: Cc1ncc2c(c1-c1ccccc1)n(Cc1ccc(S(N)(=O)=O)cc1)c(=O)n2C] |
| 21 | 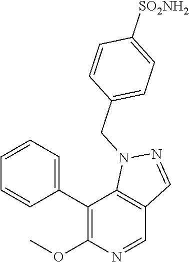 [SMILES: COc1ncc2cnn(Cc3ccc(S(N)(=O)=O)cc3)c2c1-c1ccccc1] |
| 22 | 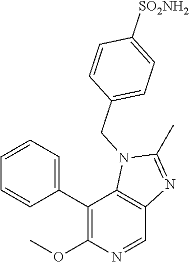 [SMILES: COc1ncc2nc(C)n(Cc3ccc(S(N)(=O)=O)cc3)c2c1-c1ccccc1] |
| 23 | 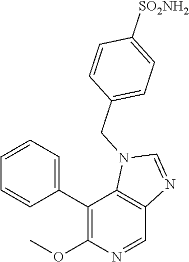 [SMILES: COc1ncc2ncn(Cc3ccc(S(N)(=O)=O)cc3)c2c1-c1ccccc1] |
| 24 | 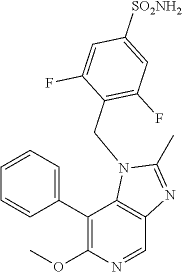 [SMILES: COc1ncc2nc(C)n(Cc3c(F)cc(S(N)(=O)=O)cc3F)c2c1-c1ccccc1] |
| 25 | 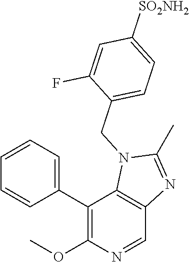 [SMILES: COc1ncc2nc(C)n(Cc3ccc(S(N)(=O)=O)cc3F)c2c1-c1ccccc1] |
| 26 | 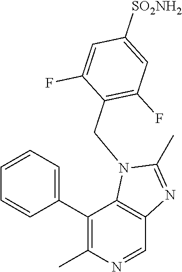 [SMILES: Cc1ncc2nc(C)n(Cc3c(F)cc(S(N)(=O)=O)cc3F)c2c1-c1ccccc1] |
| 27 | 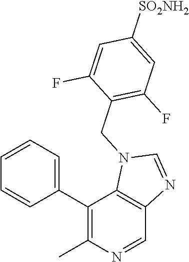 [SMILES: Cc1ncc2ncn(Cc3c(F)cc(S(N)(=O)=O)cc3F)c2c1-c1ccccc1] |
| 28 | 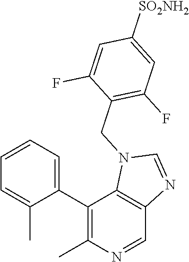 [SMILES: Cc1ccccc1-c1c(C)ncc2ncn(Cc3c(F)cc(S(N)(=O)=O)cc3F)c12] |
| 29 | 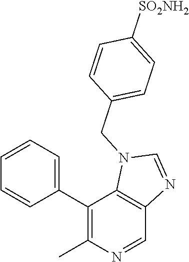 [SMILES: Cc1ncc2ncn(Cc3ccc(S(N)(=O)=O)cc3)c2c1-c1ccccc1] |
| 30 | 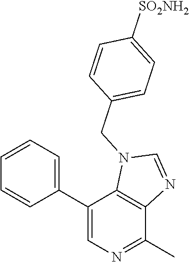 [SMILES: Cc1ncc(-c2ccccc2)c2c1ncn2Cc1ccc(S(N)(=O)=O)cc1] |
| 31 | 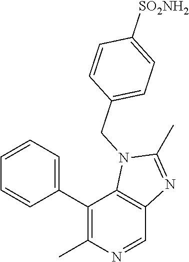 [SMILES: Cc1ncc2nc(C)n(Cc3ccc(S(N)(=O)=O)cc3)c2c1-c1ccccc1] |
| 32 | 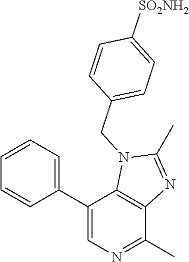 [SMILES: Cc1ncc(-c2ccccc2)c2c1nc(C)n2Cc1ccc(S(N)(=O)=O)cc1] |
| 33 | 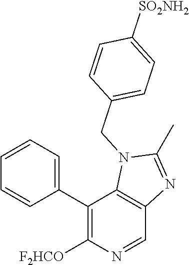 [SMILES: Cc1nc2cnc(OC(F)F)c(-c3ccccc3)c2n1Cc1ccc(S(N)(=O)=O)cc1] |
| 34 | 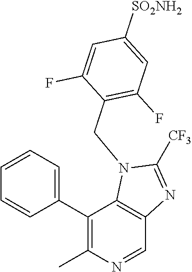 [SMILES: Cc1ncc2nc(C(F)(F)F)n(Cc3c(F)cc(S(N)(=O)=O)cc3F)c2c1-c1ccccc1] |
| 35 | 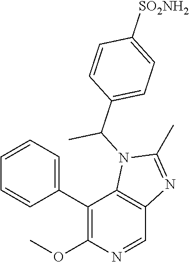 [SMILES: COc1ncc2nc(C)n(C(C)c3ccc(S(N)(=O)=O)cc3)c2c1-c1ccccc1] |
| 36 | 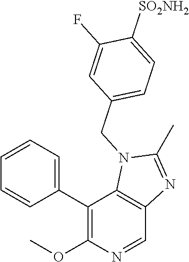 [SMILES: COc1ncc2nc(C)n(Cc3ccc(S(N)(=O)=O)c(F)c3)c2c1-c1ccccc1] |
| 37 | 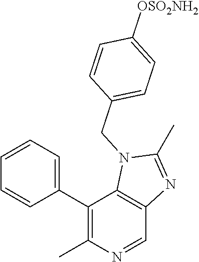 |
| 38 | 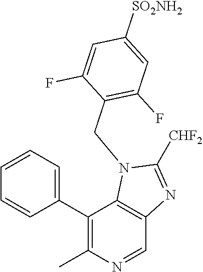 [SMILES: Cc1ncc2nc(C(F)F)n(Cc3c(F)cc(S(N)(=O)=O)cc3F)c2c1-c1ccccc1] |
| 39 | 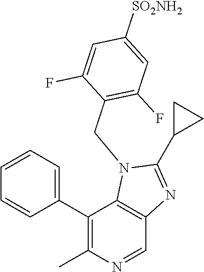 [SMILES: Cc1ncc2nc(C3CC3)n(Cc3c(F)cc(S(N)(=O)=O)cc3F)c2c1-c1ccccc1] |
| 40 | 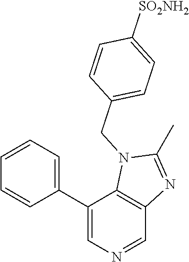 [SMILES: Cc1nc2cncc(-c3ccccc3)c2n1Cc1ccc(S(N)(=O)=O)cc1] |
| 41 | 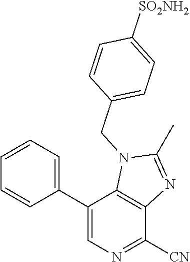 [SMILES: Cc1nc2c(C#N)ncc(-c3ccccc3)c2n1Cc1ccc(S(N)(=O)=O)cc1] |
| 42 | 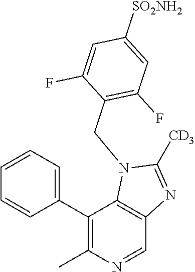 [SMILES: [2H]C([2H])([2H])c1nc2cnc(C)c(-c3ccccc3)c2n1Cc1c(F)cc(S(N)(=O)=O)cc1F] |
| 43 | 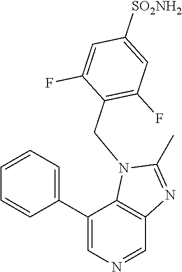 [SMILES: Cc1nc2cncc(-c3ccccc3)c2n1Cc1c(F)cc(S(N)(=O)=O)cc1F] |
| 44 | 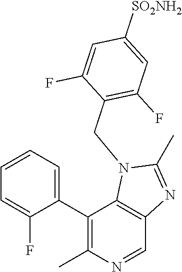 [SMILES: Cc1ncc2nc(C)n(Cc3c(F)cc(S(N)(=O)=O)cc3F)c2c1-c1ccccc1F] |
| 45 | 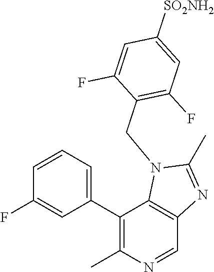 [SMILES: Cc1ncc2nc(C)n(Cc3c(F)cc(S(N)(=O)=O)cc3F)c2c1-c1cccc(F)c1] |
| 46 | 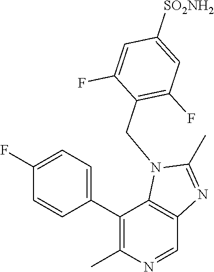 [SMILES: Cc1ncc2nc(C)n(Cc3c(F)cc(S(N)(=O)=O)cc3F)c2c1-c1ccc(F)cc1] |
| 47 | 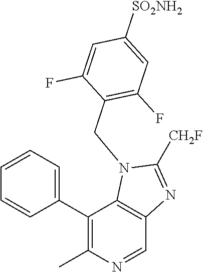 |
| 48 | 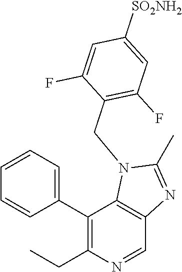 [SMILES: CCc1ncc2nc(C)n(Cc3c(F)cc(S(N)(=O)=O)cc3F)c2c1-c1ccccc1] |
| 49 | 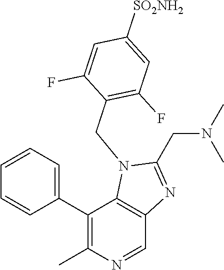 [SMILES: Cc1ncc2nc(CN(C)C)n(Cc3c(F)cc(S(N)(=O)=O)cc3F)c2c1-c1ccccc1] |
| 50 | 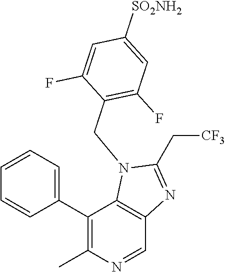 [SMILES: Cc1ncc2nc(CC(F)(F)F)n(Cc3c(F)cc(S(N)(=O)=O)cc3F)c2c1-c1ccccc1] |
| 51 | 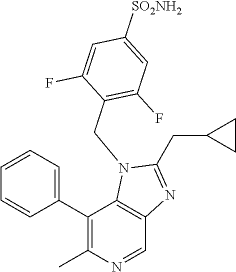 [SMILES: Cc1ncc2nc(CC3CC3)n(Cc3c(F)cc(S(N)(=O)=O)cc3F)c2c1-c1ccccc1] |
| 52 | 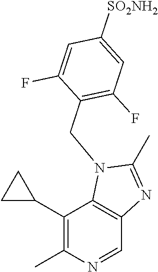 [SMILES: Cc1ncc2nc(C)n(Cc3c(F)cc(S(N)(=O)=O)cc3F)c2c1C1CC1] |
| 53 | 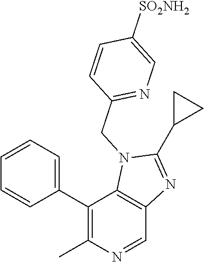 [SMILES: Cc1ncc2nc(C3CC3)n(Cc3ccc(S(N)(=O)=O)cn3)c2c1-c1ccccc1] |
| 54 | 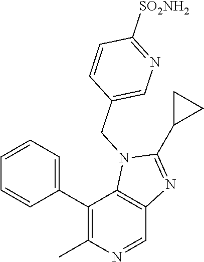 [SMILES: Cc1ncc2nc(C3CC3)n(Cc3ccc(S(N)(=O)=O)nc3)c2c1-c1ccccc1] |
| 55 | 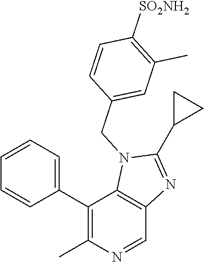 [SMILES: Cc1cc(Cn2c(C3CC3)nc3cnc(C)c(-c4ccccc4)c32)ccc1S(N)(=O)=O] |
| 56 | 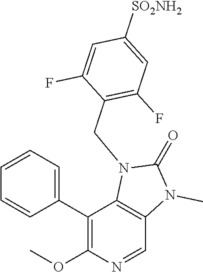 [SMILES: COc1ncc2c(c1-c1ccccc1)n(Cc1c(F)cc(S(N)(=O)=O)cc1F)c(=O)n2C] |
| 57 | 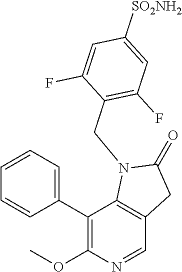 [SMILES: COc1ncc2c(c1-c1ccccc1)N(Cc1c(F)cc(S(N)(=O)=O)cc1F)C(=O)C2] |
| 58 | 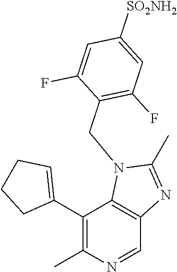 [SMILES: Cc1ncc2nc(C)n(Cc3c(F)cc(S(N)(=O)=O)cc3F)c2c1C1=CCCC1] |
| 59 | 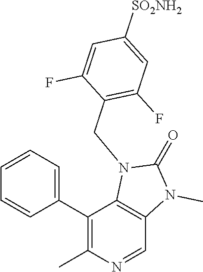 [SMILES: Cc1ncc2c(c1-c1ccccc1)n(Cc1c(F)cc(S(N)(=O)=O)cc1F)c(=O)n2C] |
| 60 | 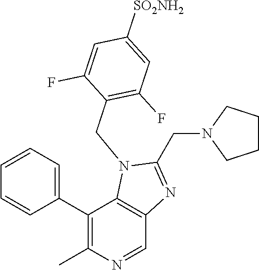 [SMILES: Cc1ncc2nc(CN3CCCC3)n(Cc3c(F)cc(S(N)(=O)=O)cc3F)c2c1-c1ccccc1] |
| 61 | 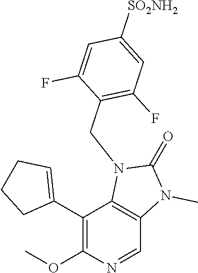 [SMILES: COc1ncc2c(c1C1=CCCC1)n(Cc1c(F)cc(S(N)(=O)=O)cc1F)c(=O)n2C] |
| 62 | 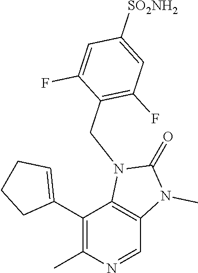 [SMILES: Cc1ncc2c(c1C1=CCCC1)n(Cc1c(F)cc(S(N)(=O)=O)cc1F)c(=O)n2C] |
| 63 | 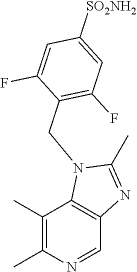 [SMILES: Cc1ncc2nc(C)n(Cc3c(F)cc(S(N)(=O)=O)cc3F)c2c1C] |
| 64 | 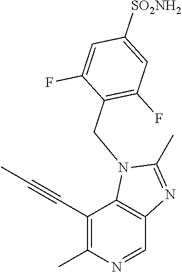 [SMILES: CC#Cc1c(C)ncc2nc(C)n(Cc3c(F)cc(S(N)(=O)=O)cc3F)c12] |
| 65 | 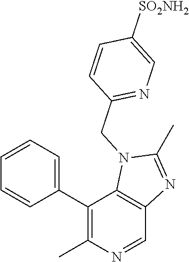 [SMILES: Cc1ncc2nc(C)n(Cc3ccc(S(N)(=O)=O)cn3)c2c1-c1ccccc1] |
| 66 | 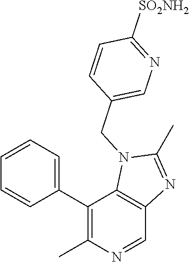 [SMILES: Cc1ncc2nc(C)n(Cc3ccc(S(N)(=O)=O)nc3)c2c1-c1ccccc1] |
| 67 | 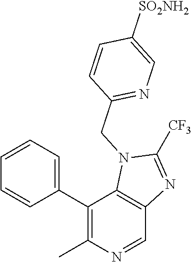 [SMILES: Cc1ncc2nc(C(F)(F)F)n(Cc3ccc(S(N)(=O)=O)cn3)c2c1-c1ccccc1] |
| 68 | 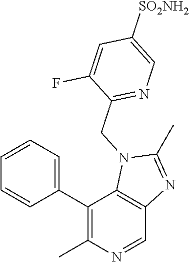 [SMILES: Cc1ncc2nc(C)n(Cc3ncc(S(N)(=O)=O)cc3F)c2c1-c1ccccc1] |
| 69 | 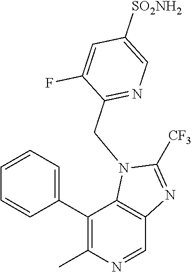 [SMILES: Cc1ncc2nc(C(F)(F)F)n(Cc3ncc(S(N)(=O)=O)cc3F)c2c1-c1ccccc1] |
| 70 | 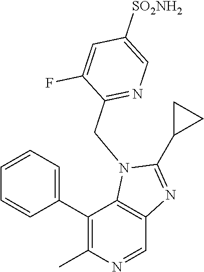 [SMILES: Cc1ncc2nc(C3CC3)n(Cc3ncc(S(N)(=O)=O)cc3F)c2c1-c1ccccc1] |
| 71 | 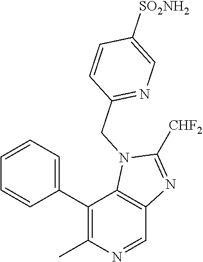 [SMILES: Cc1ncc2nc(C(F)F)n(Cc3ccc(S(N)(=O)=O)cn3)c2c1-c1ccccc1] |
| 72 | 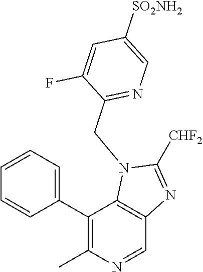 [SMILES: Cc1ncc2nc(C(F)F)n(Cc3ncc(S(N)(=O)=O)cc3F)c2c1-c1ccccc1] |
| 73 | 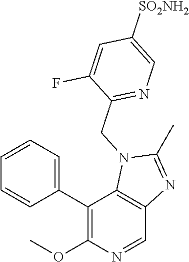 [SMILES: COc1ncc2nc(C)n(Cc3ncc(S(N)(=O)=O)cc3F)c2c1-c1ccccc1] |
| 74 | 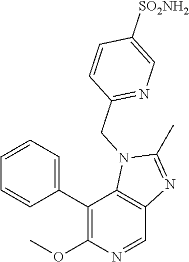 [SMILES: COc1ncc2nc(C)n(Cc3ccc(S(N)(=O)=O)cn3)c2c1-c1ccccc1] |
| 75 | 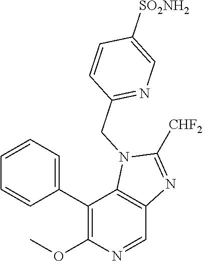 [SMILES: COc1ncc2nc(C(F)F)n(Cc3ccc(S(N)(=O)=O)cn3)c2c1-c1ccccc1] |
| 76 | 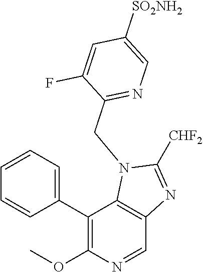 [SMILES: COc1ncc2nc(C(F)F)n(Cc3ncc(S(N)(=O)=O)cc3F)c2c1-c1ccccc1] |
| 77 | 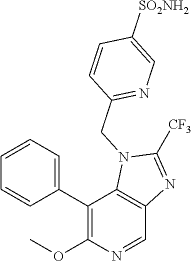 [SMILES: COc1ncc2nc(C(F)(F)F)n(Cc3ccc(S(N)(=O)=O)cn3)c2c1-c1ccccc1] |
| 78 | 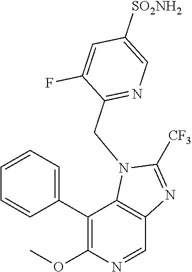 [SMILES: COc1ncc2nc(C(F)(F)F)n(Cc3ncc(S(N)(=O)=O)cc3F)c2c1-c1ccccc1] |
| 79 | 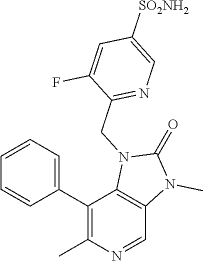 [SMILES: Cc1ncc2c(c1-c1ccccc1)n(Cc1ncc(S(N)(=O)=O)cc1F)c(=O)n2C] |
| 80 | 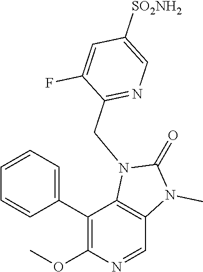 [SMILES: COc1ncc2c(c1-c1ccccc1)n(Cc1ncc(S(N)(=O)=O)cc1F)c(=O)n2C] |
| 81 | 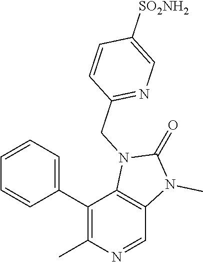 [SMILES: Cc1ncc2c(c1-c1ccccc1)n(Cc1ccc(S(N)(=O)=O)cn1)c(=O)n2C] |
| 82 | 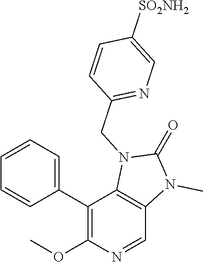 [SMILES: COc1ncc2c(c1-c1ccccc1)n(Cc1ccc(S(N)(=O)=O)cn1)c(=O)n2C] |
| 83 | 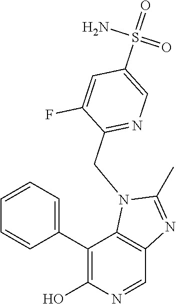 [SMILES: Cc1nc2cnc(O)c(-c3ccccc3)c2n1Cc1ncc(S(N)(=O)=O)cc1F] |
| 84 | 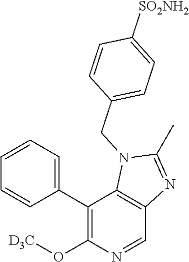 [SMILES: [2H]C([2H])([2H])Oc1ncc2nc(C)n(Cc3ccc(S(N)(=O)=O)cc3)c2c1-c1ccccc1] |
| 85 | 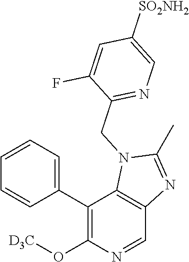 [SMILES: [2H]C([2H])([2H])Oc1ncc2nc(C)n(Cc3ncc(S(N)(=O)=O)cc3F)c2c1-c1ccccc1] |
| 86 | 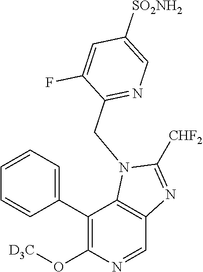 [SMILES: [2H]C([2H])([2H])Oc1ncc2nc(C(F)F)n(Cc3ncc(S(N)(=O)=O)cc3F)c2c1-c1ccccc1] |

In some embodiments the compound disclosed herein, or a pharmaceutically acceptable salt thereof, is one of the following compounds:

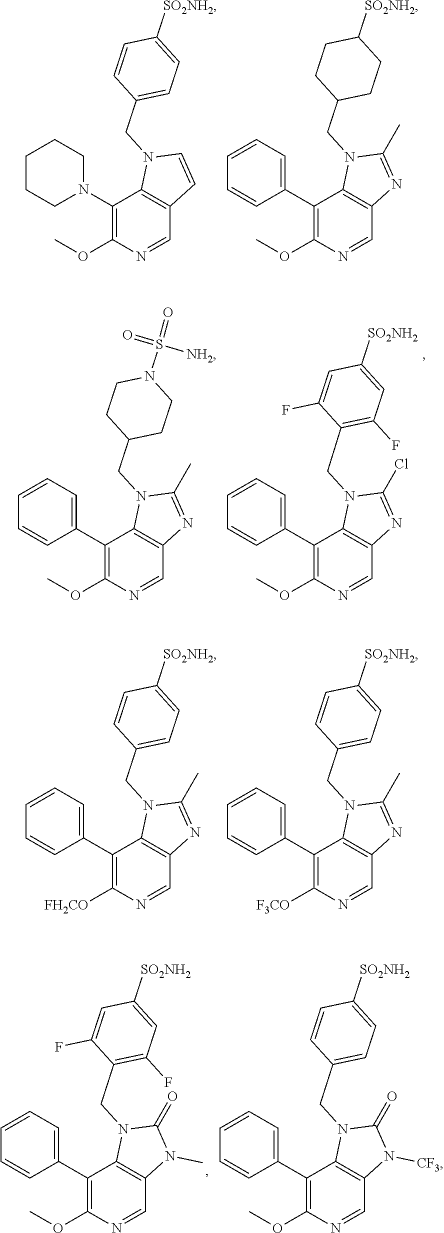

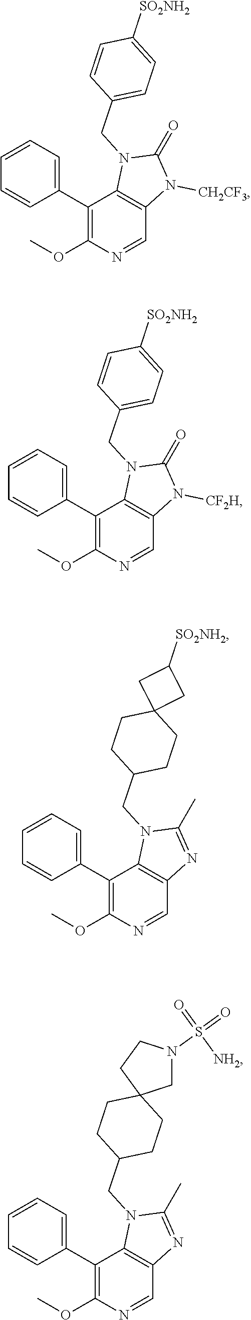

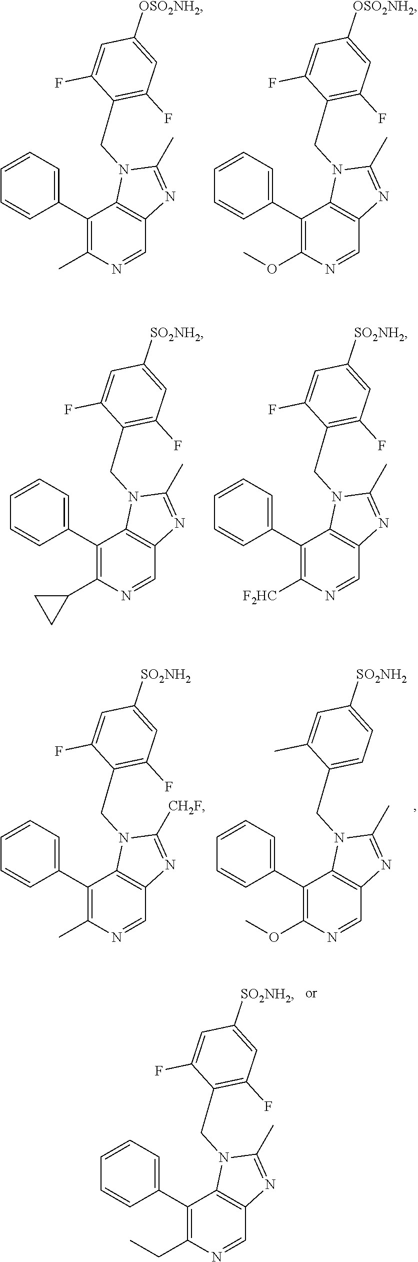

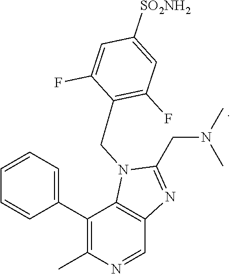 [SMILES: Cc1ncc2nc(CN(C)C)n(Cc3c(F)cc(S(N)(=O)=O)cc3F)c2c1-c1ccccc1]

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H(D), ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% by molar. In some embodiments, one or more of the substituents disclosed herein comprise deuterium at a percentage higher than the natural abundance of deuterium. In some embodiments, one or more 1H are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. In some embodiments, the disclosure provides for methods of treating diseases by administering the compounds in the form of such solvates. In some embodiments, the disclosure provides for methods of treating diseases by administering a composition comprising the compounds in the form of such solvates. Solvates contain either stoichiometric or non-stoichiometric amounts of a solvent, and, in some embodiments, are formed during the process of crystallization with pharmaceutically acceptable solvents.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein are methods of treating a disease modulated at least in part by ENPP1 in a subject in need thereof, comprising administering to the subject a therapeutically affective amount of a compound, or a pharmaceutically acceptable salt thereof, disclosed herein.

Disclosed herein are methods of treating cancer in a subject, the method comprising administering to the subject a therapeutically affective amount of a compound, or a pharmaceutically acceptable salt thereof, disclosed herein.

Disclosed herein are methods of inhibiting ENPP1 in a subject in need thereof, the method comprising administering to the subject an amount of a compound, or a pharmaceutically acceptable salt thereof. Disclosed herein are methods of regulating the STING pathway in a subject in need thereof by hydrolyzing cGAMP and/or generating immune suppressor adenosine, the method comprising administering to the subject an amount of a compound, or a pharmaceutically acceptable salt thereof. In some embodiments, the subject has cancer.

In some embodiments, the cancer is a solid tumor. In some embodiments, the solid tumor is breast cancer, lung cancer, ovarian cancer, head and neck cancer, melanoma, pancreatic cancer, liver cancer, gastric cancer, colorectal cancer, or sarcoma.

In some embodiments, the cancer is a hematologic malignancy. In some embodiments, the hematologic malignancy is a leukemia, a lymphoma, or a myeloma. In some embodiments, the hematologic malignancy is a B-cell malignancy. In some embodiments, the hematologic malignancy is multiple myeloma.

In some embodiments, the cancer is a relapsed or refractory cancer. In some embodiments, the cancer is a metastatic cancer.

In some embodiment, the cancer is hepatocellular carcinomas, glioblastomas, melanomas, testicular, pancreatic, thyroid, or breast cancer. In some embodiments, the cancer is basal cell carcinoma, biliary tract cancer, bone cancer, brain cancer (e.g., glioblastoma multiforme, glioma, medulloblastoma, primitive neuroectodermal tumor (PNET), acoustic neuroma, glioma, meningioma, pituitary adenoma, schwannoma, CNS lymphoma, primitive neuroectodermal tumor, craniopharyngioma, chordoma, medulloblastoma, cerebral neuroblastoma, central neurocytoma, pineocytoma, pineoblastoma, atypical teratoid rhabdoid tumor, chondrosarcoma, chondroma, choroid plexus carcinoma, choroid plexus papilloma, craniopharyngioma, dysembryoplastic neuroepithelial tumor, gangliocytoma, germinoma, hemangioblastoma, hemangiopercytoma, metastatic brain tumor, ependymoma, astrocytoma, oligodendroglioma, oligoastrocytoma, juvenile pilocytic astrocytoma, subependymal giant cell astrocytoma, ganglioglioma, subependymoma, pleomorphic xanthoastrocytom, anaplastic astrocytoma, glioblastoma multiforme, brain stem glioma, oligodendroglioma, ependymoma, oligoastrocytoma, cerebellar astrocytoma, desmoplastic infantile astrocytoma, subependymal giant cell astrocytoma, diffuse astrocytoma, mixed glioma, optic glioma, gliomatosis cerebri, multifocal gliomatous tumor, multicentric glioblastoma multiforme tumor, paraganglioma, ganglioglioma), breast cancer, cancer of the head and neck, cancer of the respiratory system, cancer of the urinary system, choriocarcinoma, colon cancer, connective tissue cancer, endometrial cancer, esophageal cancer, eye cancer, gastric cancer, gastrointestinal tract cancer, genitourinary tract cancer, hematological cancer (e.g., acute myeloid leukemia (AML), acute lymphoblastic leukemia (ALL), chronic myeloid leukemia (CML), chronic lymphocytic leukemia (CLL), hairy cell leukemia, chronic myelomonocytic leukemia (CMML), juvenile myelomonocytic leukemia (JMML), Hodgkin lymphoma, Non-Hodgkin lymphoma, multiple myeloma, solitary myeloma, localized myeloma, extramedullary myeloma, small lymphocytic lymphoma, B-cell non-Hodgkin lymphoma, and large B-cell lymphoma), intra-epithelial neoplasm, kidney cancer, larynx cancer, lung cancer, lymphatic system cancer, melanoma, myeloma, neuroblastoma, oral cavity cancer (e.g. lip, tongue, mouth, and pharynx), ovarian cancer, pancreatic cancer, prostate cancer, rectum cancer, rhabdomyosarcoma, sarcoma, skin cancer, stomach cancer, testicular cancer, or uterine cancer.

Disclosed herein are methods of treating an infection in a subject in need thereof comprising administering a therapeutically affective amount of a compound disclosed herein. In some embodiments, the infection is a viral infection. In some embodiments, the viral infection is due to a DNA virus. In some embodiments, the viral infection is due to a herpesvirus. In some embodiments, the herpesvirus is selected from herpes simplex viruses 1 (HSV-1), herpes simplex viruses 2 (HSV-2), varicella-zoster virus (VZV), Epstein-Barr virus (EBV), human cytomegalovirus (HCMV), human herpesvirus 6A (HHV-6A), human herpesvirus 6B (HHV-6B), human herpesvirus 7 (HHV-7), and Kaposi's sarcoma-associated herpesvirus (KSHV). In some embodiments, the herpesvirus is herpes simplex viruses 1 (HSV-1). In some embodiments, the viral infection is due to a retrovirus. In some embodiments, the retrovirus is human immunodeficiency virus (HIV). In some embodiments, the retrovirus is HIV-1, HIV-2, or human T-lymphotropic virus (HTLV). In some embodiments, the viral infection is due to a hepatitis virus. In some embodiments, the hepatitis virus is hepatitis B virus (HBV) or hepatitis D virus (HDV). In some embodiments, the viral infection is due to vaccinia virus (VACV), adenovirus, or human papillomaviruses (HPV). In some embodiments, the viral infection is due to a RNA virus. In some embodiments, the viral infection is due to dengue fever virus, yellow fever virus, Ebola virus, Marburg virus, Venezuelan encephalitis virus, or zika virus.

### Dosing

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of or risk factor for the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage, or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the subject every 12 hours; (v) the compound is administered to the subject every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In one embodiment, the compounds of this disclosure may be administered to animals. The compounds can be administered orally or parenterally, including the intravenous, intramuscular, intraperitoneal, subcutaneous, rectal, and topical routes of administration.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### Combination

Disclosed herein are methods of treating a disease or disorder associated with ENPP1 using a compound disclosed herein, or a pharmaceutically acceptable salt thereof, in combination with an additional therapeutic agent.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

In some embodiments, the additional therapeutic agent is an anti-cancer agent.

In some embodiments, the additional therapeutic agent is an ALK inhibitor: e.g. crizotinib, NVP-TAE684, ceritinib, alectinib, brigatinib, entrecinib, or lorlatinib; an androgen receptor inhibitor: e.g. enzalutamide, apalutamide, abiraterone acetate, orteronel, galeterone, seviteronel, bicalutamide, or flutamide; an antineoplastic agent: e.g. oxaliplatin, carboplatin, or cisplatin; an aromatase inhibitor: exemestane, letrozole, anastrozole, fulvestrant, or tamoxifen; a BCL-2 inhibitor: e.g. venetoclax; a BCR-ABL inhibitor: e.g. imatinib, inilotinib, nilotinib, dasatinib, bosutinib, ponatinib, bafetinib, danusertib, saracatinib, or PF03814735; a BRAF inhibitor: e.g. vemurafenib or dabrafenib; a CD20 antibody: e.g. rituximab, tositumomab, or ofatumumab; a CDK4/6 inhibitor: e.g. alvocidib, palbociclib, ribociclib, trilaciclib, or abemaciclib; a CTLA-4 inhibitor: e.g. tremelimumab or ipilimumab; a DNA synthesis inhibitor: e.g. capecitabine, gemcitabine, nelarabine, or hydroxycarbamide; an epidermal growth factor receptor (EGFR) inhibitor: e.g. gefitinib, osimertinib, cetuximab, or panitumumab; an ERK inhibitor: e.g. ulixertinib, MK 8353, or LY 3214996; a KRAS inhibitor: e.g. AMG-510, MRTX849, or ARS-3248; an FGFR inhibitor: e.g. infigratinib, dovitinib, erdafitinib, TAS-120, pemigatinib, BLU-554, or AZD4547; an FLT3 inhibitor: e.g. sunitinib, midostaurin, tanutinib, sorafenib, lestaurtinib, quizartinib, or crenolanib; a Heat Shock Protein (HSP) inhibitor: e.g. tanespimycin; a Hedgehog antagonist: e.g. vismodegib; an HER2 receptor inhibitor: e.g. trastuzumab, pertuzumab, neratinib, lapatinib, or lapatinib; a Histone deacetylase inhibitor (HDI): e.g. vorinostat; Immunomodulators: e.g. afutuzumab, lenalidomide, thalidomide, or pomalidomide; a CD40 inhibitor: e.g. dacetuzumab; a MEK inhibitor: e.g. trametinib, cobimetinib, binimetinib, or selumetinib; a MET inhibitor: e.g. crizotinib or cabozantinib; an mTOR inhibitor: e.g. temsirolimus, ridaforolimus, everolimus, or sirolimus; a PD1 inhibitor: e.g. nivolumab, or pembrolizumab; a PDL1 inhibitor: e.g. MSB0010718C; YW243.55.S70, MPDL3280A, MEDI-4736, MSB-0010718C, or MDX-1105; a PI3K inhibitor: e.g. pictilisib, dactolisib, alpelisib, buparlisib, taselisib, idelalisib, duvelisib, or umbralisib; a PIK3CA inhibitor; a Pro-apoptotic receptor agonists (PARAs): e.g. dulanermin; a proteasome inhibitor: e.g. bortezomib; a SHP2 inhibitor; or a Tyrosine kinase inhibitor: e.g. erlotinib, linifanib, sunitinib, or pazopanib.

### Examples

### Example 1: Synthesis of N-(4-((6-methoxy-3-methyl-7-(1-methyl-1H-pyrazol-3-yl)-1H-pyrrolo[3,2-c]pyridin-1-yl)methyl)phenyl) sulfamide

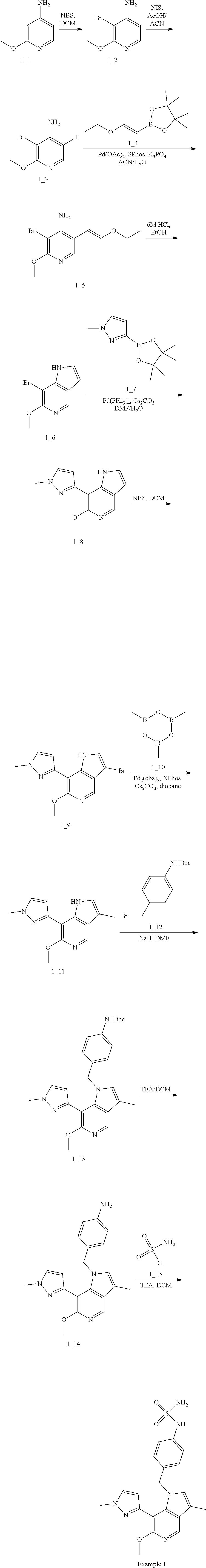

### Step 1: General procedure for preparation of 3-bromo-2-methoxypyridin-4-amine

To a solution of Compound 1_1 (5 g, 40.3 mmol) in DCM (100 mL) was added NBS (7.17 g, 40.3 mmol) at 0° C. The mixture was stirred at room temperature for 2 h. After the solvent was removed under reduced pressure, the obtained residue was purified by flash silica gel chromatography to provide the corresponding Compound 1_2 (7 g, 85.9% yield) as a yellow solid. LCMS: [M+H]⁺=203.0. ¹H NMR: (400 MHZ, DMSO-d₆) δ 7.60 (d, J=5.6 Hz, 1H), 6.37 (d, J=5.6 Hz, 1H), 6.20 (s, 2H), 3.81 (s, 3H).

### Step 2: General procedure for preparation of 3-bromo-5-iodo-2-methoxypyridin-4-amine (Compound 1_3)

To a solution of Compound 1_2 (7 g, 34.7 mmol) in MeCN/AcOH(70 mL/7 mL) was added NIS (8.60 g, 38.2 mmol) at 0° C. The mixture was stirred at room temperature for 16 h. After the solvent was removed under reduced pressure, the obtained residue was diluted with water (100 mL). The aqueous phase was extracted with DCM (100 mL×2). The combined organic phase was washed by brine (100 mL), dried over anhydrous sodium sulfate, filtered, and concentrated in vacuum to provide the corresponding Compound 1_3 (10.3 g, 90% yield) as a yellow solid. The crude product was directly used for the next step. LCMS: [M+H]⁺=328.8.

### Step 3: General procedure for preparation of (E)-3-bromo-5-(2-ethoxyvinyl)-2-methoxypyridin-4-amine (Compound 1_5)

A mixture of Compound 1_3 (10 g, 30.5 mmol), Compound 1_4 (6.65 g, 33.6 mmol), K₃PO₄ (19.40 g, 91.5 mmol), SPhos (1.3 g, 3.1 mmol) and Pd(OAc)₂ (697.5 mg, 3.1 mmol) in MeCN/H₂O (80 mL/20 mL) was stirred at 110° C. for 10 h under N₂ atmosphere. After the solvent was removed under reduced pressure, the obtained residue was purified by flash silica gel chromatography to provide the corresponding Compound 1_5 (3.5 g, 42% yield) as a yellow solid. LCMS: [M+H]⁺=273.0.

### Step 4: General procedure for preparation of 7-bromo-6-methoxy-1H-pyrrolo[3,2-c]pyridine (Compound 1_6)

To a solution of Compound 1_5 (3.5 g, 12.9 mmol) in EtOH(20 mL) was added concentrated hydrochloric acid (20 mL). The mixture was stirred at 80° C. for 16 h. Solvent was removed under reduced pressure to provide the corresponding Compound 1_6 (2 g, 69.0% yield) as a brown solid. The crude product was directly used for the next step.

LCMS: [M+H]⁺=227.0.

### Step 5: General procedure for preparation of 6-methoxy-7-(1-methyl-1H-pyrazol-3-yl)-1H-pyrrolo[3,2-c]pyridine (Compound 1_8)

A mixture of Compound 1_6 (2 g, 8.85 mmol), Compound 1_7 (2.75 g, 13.2 mmol), Cs₂CO₃ (8.66 g, 26.55 mmol) and Pd(PPh₃)₄ (1.03 g, 0.89 mmol) in DMF (20 mL) and H₂O (5 mL) was stirred at 90° C. for 16 h under N₂ atmosphere. After the resulting mixture was concentrated under reduced pressure, the obtained residue purified by flash silica gel chromatography to provide the corresponding Compound 1_8 (500 mg, 25.0% yield) as a purple solid. LCMS: [M+H]⁺=229.1. ¹H NMR: (400 MHZ, DMSO-d₆) δ 11.04 (s, 1H), 8.39 (s, 1H), 7.80 (d, J=2.4 Hz, 1H), 7.38-7.30 (m, 1H), 6.90 (d, J=2.4 Hz, 1H), 6.55-C_{6.49} (m, 1H), 4.00 (s, 6H).

### Step 6: General procedure for preparation of 3-bromo-6-methoxy-7-(1-methyl-1H-pyrazol-3-yl)-1H-pyrrolo[3,2-c]pyridine (Compound 1_9)

To a solution of Compound 1_8 (500 mg, 2.19 mmol) in DCM (10 mL) was added NBS (312 mg, 1.75 mmol) at 0° C. The reaction mixture was stirred at 0° C. for 2 s. After the solvent was removed under reduced pressure, the obtained residue purified by flash silica gel chromatography to provide the corresponding Compound 1_9 (180 mg, 26.9% yield) as a yellow solid. LCMS: [M+H]⁺=307.0.

### Step 7: General procedure for preparation of 6-methoxy-3-methyl-7-(1-methyl-1H-pyrazol-3-yl)-1H-pyrrolo[3,2-c]pyridine (Compound 1_11)

A mixture of Compound 1_9 (180 mg, 0.59 mmol), Compound 1_10 (0.5 mL), Cs₂CO₃ (577 mg, 1.77 mmol), XPhos (87 mg, 0.18 mmol) and Pd₂ (dba) 3 (55 mg, 0.06 mmol) in dioxane (10 mL) was stirred at 110° C. for 16 h under N₂ atmosphere. After the solvent was removed under reduced pressure, the obtained residue purified by flash silica gel chromatography to provide the corresponding Compound 1_11 (120 mg) as a yellow solid. LCMS: [M+H]⁺=243.1.

### Step 8: General procedure for preparation of tert-butyl (4-((6-methoxy-3-methyl-7-(1-methyl-1H-pyrazol-3-yl)-1H-pyrrolo[3,2-c]pyridin-1-yl)methyl)phenyl) carbamate (Compound 1_13)

To a solution of Compound 1_11 (120 mg, 0.49 mmol) in DMF (5 mL) was added NaH(59 mg, 1.47 mmol, 60% in oil) at 0° C. The reaction mixture was stirred at this temperature for 0.5 h under N₂ atmosphere. Then Compound 1_12 (211 mg, 0.74 mmol) was added. The reaction mixture was stirred at room temperature for 2 h. Then the reaction mixture was diluted with water (30 mL). The aqueous phase was extracted with ethyl acetate (50 mL×2). The combined organic phase was washed by brine (30 mL), dried over anhydrous sodium sulfate, filtered, and concentrated in vacuum. The obtained residue was purified by flash silica gel chromatography to provide the corresponding Compound 1_13 (80 mg) as a yellow solid. LCMS: [M+H]⁺=448.3.

### Step 9: General procedure for preparation of 4-((6-methoxy-3-methyl-7-(1-methyl-1H-pyrazol-3-yl)-1H-pyrrolo[3,2-c]pyridin-1-yl)methyl) aniline (Compound 1_14)

A solution of Compound 1_13 (80 mg, 0.18 mmol) in DCM (5 mL) and TFA (1 mL) was stirred at room temperature for 2 h. After the solvent was removed under reduced pressure, the obtained residue was purified by Prep-HPLC to afford the corresponding Compound 1_14 (40 mg, 64.5% yield) as a white solid. LCMS: [M+H]⁺=348.2.

### Step 10: General procedure for preparation of N-(4-((6-methoxy-3-methyl-7-(1-methyl-1H-pyrazol-3-yl)-1H-pyrrolo[3,2-c]pyridin-1-yl)methyl)phenyl) sulfamide

To a solution of Compound 1_14 (40 mg, 0.12 mmol) in DCM (5 mL) were added Compound 1_15 (28 mg, 0.24 mmol) and TEA (36 mg, 0.36 mmol). The reaction was stirred at room temperature for 2 h. After the solvent was removed under reduced pressure, the obtained residue was purified by Prep-HPLC to provide Example 1 (1.5 mg, 2.9% yield). LCMS: [M+H]⁺=427. ¹H NMR: (400 MHZ, DMSO-d₆) δ 9.36 (s, 1H), 8.37 (s, 1H), 7.69 (d, J=2.0 Hz, 1H), 7.02-C_{6.96} (m, 3H), 6.93 (d, J=8.4 Hz, 2H), 6.65 (d, J=8.4 Hz, 2H), 6.20 (d, J=2.0 Hz, 1H), 4.83 (s, 2H), 3.86 (s, 3H), 3.78 (s, 3H), 2.25 (s, 3H).

### Example 2: Synthesis of N-(4-((6-methyl-7-(1-methyl-1H-pyrazol-3-yl)-1H-pyrrolo[3,2-c]pyridin-1-yl)methyl)phenyl) sulfamide

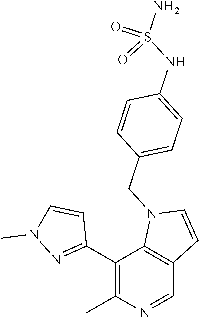 [SMILES: Cc1ncc2ccn(Cc3ccc(NS(N)(=O)=O)cc3)c2c1-c1ccn(C)n1]

The title compound was synthesized as described in example 1. LCMS: [M+H]⁺=397.1. ¹H NMR: (400 MHZ, DMSO-d₆) δ 9.39 (s, 1H), 8.71 (s, 1H), 7.77 (d, J=2.0 Hz, 1H), 7.31 (d, J=3.2 Hz, 1H), 7.01 (s, 2H), 6.96 (d, J=8.4 Hz, 2H), 6.67-C_{6.61} (m, 3H), 6.21 (d, J=2.0 Hz, 1H), 4.83 (s, 2H), 3.86 (s, 3H), 2.28 (s, 3H).

### Example 3: Synthesis of N-(4-((6-methoxy-7-(1-methyl-1H-pyrazol-3-yl)-1H-pyrrolo[3,2-c]pyridin-1-yl)methyl)phenyl) sulfamide

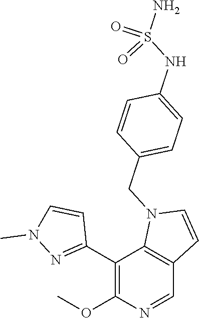 [SMILES: COc1ncc2ccn(Cc3ccc(NS(N)(=O)=O)cc3)c2c1-c1ccn(C)n1]

The title compound was synthesized as described in example 1. LCMS: [M+H]⁺=413.1. ¹H NMR: (400 MHz, DMSO-d₆) δ 8.44 (s, 1H), 7.69 (d, J=2.0 Hz, 1H), 7.28 (d, J=3.2 Hz, 1H), 6.93 (d, J=8.4 Hz, 2H), 6.65-C_{6.55} (m, 3H), 6.18 (d, J=2.0 Hz, 1H), 6.05 (s, 1H), 4.94 (s, 2H), 3.87 (s, 3H), 3.77 (s, 3H).

### Example 4: Synthesis of 3,5-difluoro-4-((6-methoxy-3-methyl-7-(1-methyl-1H-pyrazol-3-yl)-1H-pyrrolo[3,2-c]pyridin-1-yl)methyl)benzene sulfonamide

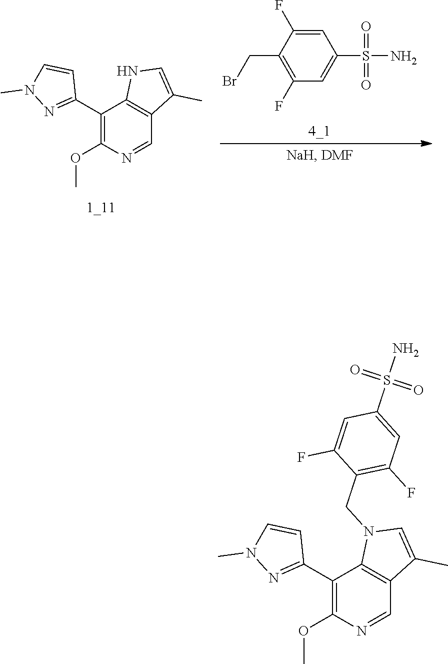

To a solution of Compound 1_11 (90 mg, 0.37 mmol) in DMF (5 mL) was added NaH(44 mg, 1.11 mmol, 60% in oil) at 0° C. The mixture was stirred at 0° C. for 0.5 h under N₂ atmosphere. Then Compound 4_1 (116 mg, 0.41 mmol) was added. The reaction mixture was stirred at room temperature for 2 h. After the reaction mixture was diluted with water (30 mL), the aqueous phase was extracted with ethyl acetate (50 mL×2). The combined organic phase was washed by brine (10 mL), dried over anhydrous sodium sulfate, filtered, and concentrated in vacuum. The obtained residue purified by Prep-HPLC to afford example 4 (17 mg, 10.3%). LCMS: [M+H]⁺=448.1. ¹H NMR: (400 MHZ, DMSO-d₆) δ 8.38 (s, 1H), 7.61 (d, J=2.4 Hz, 1H), 7.58 (s, 2H), 7.31 (d, J=7.2 Hz, 2H), 6.90 (s, 1H), 6.11 (d, J=2.4 Hz, 1H), 5.13 (s, 2H), 3.81 (s, 3H), 3.77 (s, 3H), 2.25 (s, 3H).

### Example 5: Synthesis of 4-((6-methoxy-7-(1-methyl-1H-pyrazol-3-yl)-1H-pyrrolo[3,2-c]pyridin-1-yl)methyl)benzenesulfonamide

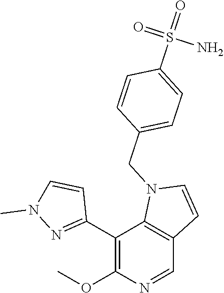 [SMILES: COc1ncc2ccn(Cc3ccc(S(N)(=O)=O)cc3)c2c1-c1ccn(C)n1]

The title compound was synthesized as described in example 4. LCMS: [M+H]⁺=398.1. ¹H NMR: (400 MHZ, DMSO-d₆) δ 8.48 (s, 1H), 7.62-7.56 (m, 3H), 7.36 (d, J=3.2 Hz, 1H), 7.24 (s, 2H), 6.72 (d, J=8.4 Hz, 2H), 6.64 (d, J=3.2 Hz, 1H), 6.04 (d, J=2.0 Hz, 1H), 5.16 (s, 2H), 3.80 (s, 3H), 3.76 (s, 3H).

### Example 6: Synthesis of 4-((6-methoxy-3-methyl-7-(1-methyl-1H-pyrazol-3-yl)-1H-pyrrolo[3,2-c]pyridin-1-yl)methyl)benzenesulfonamide

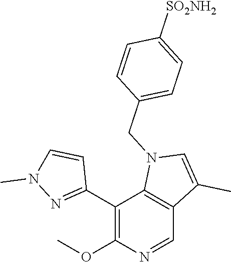

The title compound was synthesized as described in example 4. LCMS: [M+H]⁺=412.1. ¹H NMR: (400 MHZ, DMSO-d₆) δ 8.41 (s, 1H), 7.62-7.54 (m, 3H), 7.23 (s, 2H), 7.07 (s, 1H), 6.76 (d, J=8.4 Hz, 2H), 6.05 (d, J=2.0 Hz, 1H), 5.05 (s, 2H), 3.79 (s, 3H), 3.77 (s, 3H), 2.29 (s, 3H).

### Example 7: Synthesis of 3,5-difluoro-4-((6-methoxy-7-(1-methyl-1H-pyrazol-3-yl)-1H-pyrrolo[3,2-c]pyridin-1-yl)methyl)benzenesulfonamide

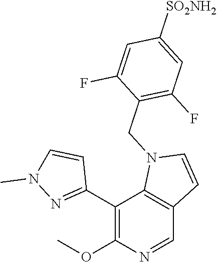 [SMILES: COc1ncc2ccn(Cc3c(F)cc(S(N)(=O)=O)cc3F)c2c1-c1ccn(C)n1]

The title compound was synthesized as described in example 4. LCMS: [M+H]⁺=434.2. ¹H NMR: (400 MHZ, DMSO-d₆) δ 8.45 (s, 1H), 7.60 (d, J=2.0 Hz, 1H), 7.57 (s, 2H), 7.29 (d, J=7.2 Hz, 2H), 7.21 (d, J=3.2 Hz, 1H), 6.58 (d, J=3.2 Hz, 1H), 6.08 (d, J=2.0 Hz, 1H), 5.26 (s, 2H), 3.81 (s, 3H), 3.77 (s, 3H).

### Example 8: Synthesis of 4-((6-methoxy-7-(3-methyl-1H-pyrazol-1-yl)-1H-pyrrolo[3,2-c]pyridin-1-yl)methyl)benzenesulfonamide

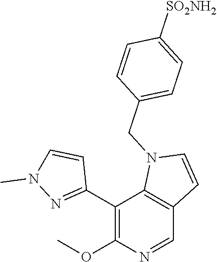 [SMILES: COc1ncc2ccn(Cc3ccc(S(N)(=O)=O)cc3)c2c1-c1ccn(C)n1]

The title compound was synthesized as described in example 4. LCMS: [M+H]⁺=398.1. ¹H NMR: (400 MHZ, CD₃OD) δ 8.53 (s, 1H), 7.71 (d, J=6.8 Hz, 2H), 7.26 (s, 1H), 7.22 (s, 1H), 6.83 (d, J=7.6 Hz, 2H), 6.73 (s, 1H), 6.12 (s, 1H), 4.83 (s, 2H), 3.88 (s, 3H), 2.23 (s, 3H).

### Example 9: Synthesis of 4-((6-methoxy-7-phenyl-1H-pyrrolo[3,2-c]pyridin-1-yl)methyl)benzene sulfonamide

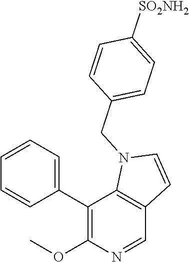 [SMILES: COc1ncc2ccn(Cc3ccc(S(N)(=O)=O)cc3)c2c1-c1ccccc1]

The title compound was synthesized as described in example 4. LCMS: [M+H]⁺=394.1. ¹H NMR: (400 MHZ, DMSO-d₆) δ 8.51 (s, 1H), 7.56 (d, J=8.4 Hz, 2H), 7.34-7.27 (m, 2H), 7.29-7.19 (m, 4H), 7.09-7.04 (m, 2H), 6.67 (d, J=3.6 Hz, 1H), 6.56 (d, J=8.0 Hz, 2H), 4.88 (s, 2H), 3.74 (s, 3H).

### Example 10: Synthesis of 4-((6-methoxy-3-methyl-7-phenyl-1H-pyrrolo[3,2-c]pyridin-1-yl)methyl)benzene sulfonamide

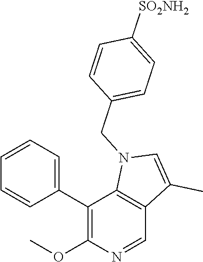 [SMILES: COc1ncc2c(C)cn(Cc3ccc(S(N)(=O)=O)cc3)c2c1-c1ccccc1]

The title compound was synthesized as described in example 4. LCMS: [M+H]⁺=408.1. ¹H NMR: (400 MHZ, CD₃OD) δ 8.56 (s, 1H), 7.64 (d, J=8.8 Hz, 2H), 7.40-7.30 (m, 1H), 7.30-7.23 (m, 2H), 7.16 (s, 1H), 7.10-7.04 (m, 2H), 6.560 (d, J=8.0 Hz, 2H), 4.94 (s, 2H), 3.86 (s, 3H).

### Example 11: Synthesis of 4-((6-methoxy-7-(1-(trifluoromethyl)-1H-pyrazol-4-yl)-1H-pyrrolo[3,2-c]pyridin-1-yl)methyl)benzenesulfonamide

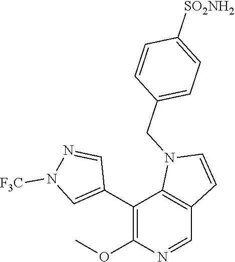 [SMILES: COc1ncc2ccn(Cc3ccc(S(N)(=O)=O)cc3)c2c1-c1cnn(C(F)(F)F)c1]

The title compound was synthesized as described in example 4. LCMS: [M+H]⁺=452.1. ¹H NMR: (400 MHZ, DMSO-d₆) δ 8.54 (s, 1H), 8.16 (s, 1H), 7.69 (s, 1H), 7.60 (d, J=8.4 Hz, 2H), 7.44 (d, J=3.2 Hz, 1H), 7.26 (s, 2H), 6.69 (d, J=3.2 Hz, 1H), 6.62 (d, J=8.4 Hz, 2H), 5.16 (s, 2H), 3.77 (s, 3H).

### Example 12: Synthesis of 4-((6-methoxy-7-(1-(trifluoromethyl)-1H-pyrazol-4-yl)-1H-pyrrolo[3,2-c]pyridin-1-yl)methyl)benzenesulfonamide

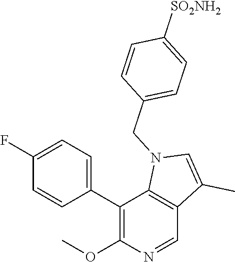 [SMILES: COc1ncc2c(C)cn(Cc3ccc(S(N)(=O)=O)cc3)c2c1-c1ccc(F)cc1]

The title compound was synthesized as described in example 4. LCMS: [M+H]⁺=426.1.

¹H NMR: (400 MHZ, DMSO-d₆) § 8.45 (s, 1H), 7.57 (d, J=8.0 Hz, 2H), 7.27 (s, 2H), 7.09-6.97 (m, 5H), 6.60 (d, J=8.0 Hz, 2H), 4.84 (s, 2H), 3.74 (s, 3H), 2.31 (s, 3H).

### Example 13: Synthesis of 4-((6-methoxy-3-methyl-7-(pyridin-2-yl)-1H-pyrrolo[3,2-c]pyridin-1-yl)methyl)benzenesulfonamide

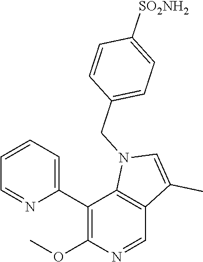 [SMILES: COc1ncc2c(C)cn(Cc3ccc(S(N)(=O)=O)cc3)c2c1-c1ccccn1]

The title compound was synthesized as described in example 4. LCMS: [M+H]⁺=409.2. ¹H NMR: (400 MHZ, DMSO-d₆) δ 8.56 (d, J=4.0 Hz, 1H), 8.49 (s, 1H), 7.57-7.47 (m, 3H), 7.32-7.20 (m, 3H), 7.10 (s, 1H), 7.03 (d, J=8.0 Hz, 1H), 6.55 (d, J=8.4 Hz, 2H), 4.89 (s, 2H), 3.76 (s, 3H), 2.32 (s, 3H).

### Example 14: Synthesis of 4-[(3,6-dimethyl-7-phenylpyrrolo[3,2-c]pyridin-1-yl)methyl]-3,5-difluorobenzene-1-sulfonamide

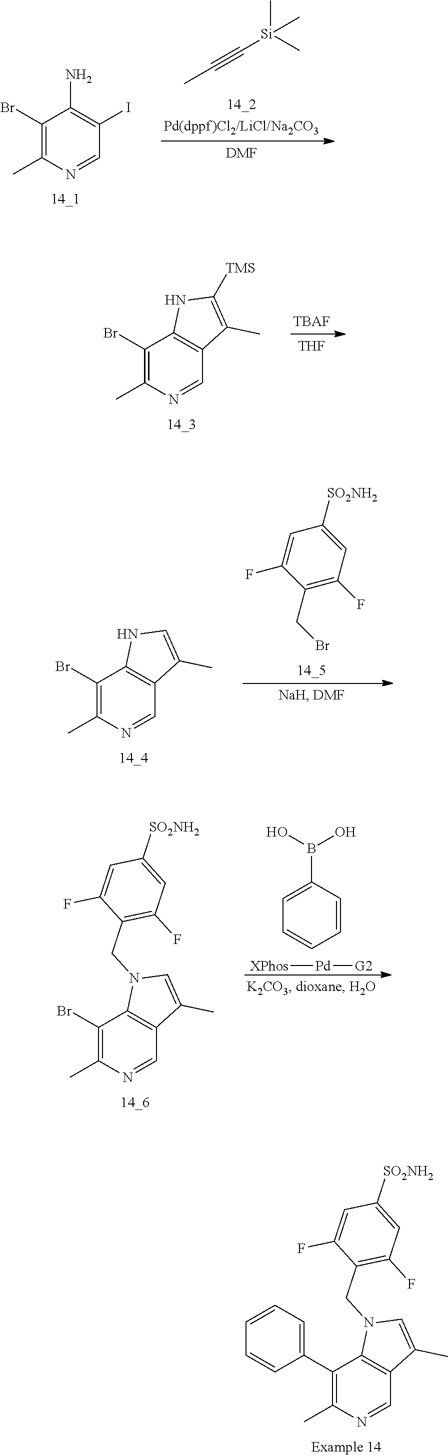

### Step 1: General procedure for preparation of 7-bromo-3,6-dimethyl-2-(trimethylsilyl)-1H-pyrrolo[3,2-c]pyridine (Compound 14_3)

To a solution of Compound 14_1 (4.8 g, 15.4 mmol) in DMF (50 mL) were added LiCl (652.26 mg, 15.4 mmol), Na₂CO₃ (1.6 g, 15.4 mmol), Pd(dppf) Cl₂ (1.12 g, 15.4 mmol) and Compound 14_2 (5.2 g, 46.2 mmol). The reaction mixture was stirred at 100° C. overnight under N₂ atmosphere. After the solvent was concentrated under reduced pressure, the obtained residue was purified by flash silica gel chromatography to provide the corresponding Compound 14_3 (2.7 g, 59.0% yield) as a yellow solid. LCMS: [M+H]⁺=299.0.

### Step 2: General procedure for preparation of 7-bromo-3,6-dimethyl-1H-pyrrolo[3,2-c]pyridine (Compound 14_4)

To a solution of Compound 14_3 (2.7 g, 9.1 mmol) in THF (30 mL) was added TBAF (7.1 g, 27.4 mmol). The reaction mixture was stirred at room temperature for 16 h. After the solvent was concentrated under reduced pressure, the obtained residue was purified by flash silica gel chromatography to provide the corresponding Compound 14_4 (1.2 g, 58.7% yield) as a white solid.

LCMS: [M+H]⁺=226.9.

### Step 3: General procedure for preparation of 4-(((3-bromo-2-methoxy-5-nitropyridin-4-yl)amino) methyl)benzenesulfonamide (Compound 14_6)

To a solution of Compound 14_4 (750.0 mg, 3.3 mmol) in DMF (8 mL) was added NaH (240.0 mg, 10.0 mmol, 60% in oil) at 0° C. The mixture was stirred at 0° C. for 30 min. Then Compound 14_5 (953.3 mg, 3.3 mmol) was added, and the reaction mixture was stirred at room temperature for 30 min. After the reaction mixture was quenched by water (10 mL). The aqueous layer was extracted with ethyl acetate (3×25 mL). The combined organic layer was washed with saturated NaCl solution (3×50 mL), dried over Na₂SO₄, filtered, and concentrated under reduced pressure. The obtained residue was purified by flash silica gel chromatography to provide the corresponding Compound 14_6 (330 mg, 23.0% yield) as a yellow solid. LCMS: [M+H]⁺=429.9.

### Step 4: General procedure for preparation of 4-[(3,6-dimethyl-7-phenylpyrrolo[3,2-c]pyridin-1-yl)methyl]-3,5-difluorobenzene-1-sulfonamide

To a solution of Compound 14_6 (220.0 mg, 0.5 mmol) in dioxane (5 mL) and H₂O (0.5 mL) were added K₂CO₃ (212.0 mg, 1.5 mmol), XPhos-Pd-G2 (40.2 mg, 0.05 mmol) and phenylboronic acid (124.7 mg, 1.0 mmol). The reaction mixture was stirred at 100° C. for 16 h under N₂ atmosphere. After the solvent was concentrated under reduced pressure, the obtained residue was purified by Prep-HPLC to afford example 14 (22.7 mg, 10.4% yield). LCMS: [M+H]⁺=428.1. ¹H NMR: (400 MHZ, DMSO-d₆) δ 8.68 (s, 1H), 7.61 (s, 2H), 7.43-7.38 (m, 3H), 7.35 (d, J=7.2 Hz, 2H), 7.23 (s, 2H), 6.88 (s, 1H), 4.78 (s, 2H), 2.27 (s, 3H), 2.19 (s, 3H).

### Example 15: Synthesis of 4-((6-methyl-7-phenyl-1H-pyrrolo[3,2-c]pyridin-1-yl)methyl)benzene sulfonamide

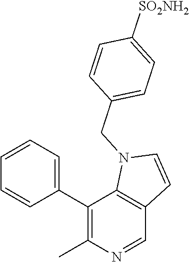 [SMILES: Cc1ncc2ccn(Cc3ccc(S(N)(=O)=O)cc3)c2c1-c1ccccc1]

The title compound was synthesized as described in example 14. LCMS: [M+H]⁺=378.1.

¹H NMR: (400 MHZ, DMSO-d₆) δ 8.77 (s, 1H), 7.58 (d, J=8.4 Hz, 2H), 7.37 (d, J=3.2 Hz, 2H), 7.32-7.25 (m, 4H), 7.07-7.02 (m, 2H), 6.73 (d, J=3.2 Hz, 1H), 6.56 (d, J=8.0 Hz, 2H), 4.87 (s, 2H), 2.16 (s, 3H).

### Example 16: Synthesis of 4-((3,6-dimethyl-7-phenyl-1H-pyrrolo[3,2-c]pyridin-1-yl)methyl)benzene sulfonamide

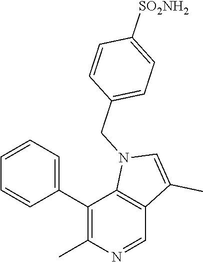 [SMILES: Cc1ncc2c(C)cn(Cc3ccc(S(N)(=O)=O)cc3)c2c1-c1ccccc1]

The title compound was synthesized as described in example 14. LCMS: [M+H]⁺=392.1. ¹H NMR: (400 MHZ, DMSO-d₆) δ 8.72 (s, 1H), 7.58 (d, J=8.4 Hz, 2H), 7.36 (t, J=7.2 Hz, 1H), 7.28 (s, 4H), 7.09-7.03 (m, 3H), 6.60 (d, J=8.4 Hz, 2H), 4.77 (s, 2H), 2.33 (s, 3H), 2.16 (s, 3H).

### Example 17: Synthesis of 4-((3,6-dimethyl-7-(1-methyl-1H-pyrazol-3-yl)-1H-pyrrolo[3,2-c]pyridin-1-yl)methyl)-3,5-difluorobenzenesulfonamide

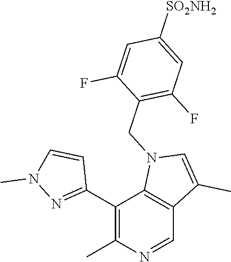

The title compound was synthesized as described in example 14. LCMS: [M+H]⁺=432.2. ¹H NMR: (400 MHZ, DMSO-d₆) § 8.66 (s, 1H), 7.72 (d, J=2.0 Hz, 1H), 7.59 (s, 2H), 7.35 (d, J=7.0 Hz, 2H), 6.94 (s, 1H), 6.18 (d, J=2.1 Hz, 1H), 5.02 (s, 2H), 3.81 (s, 3H), 2.27 (s, 6H).

### Example 18: Synthesis of 4-((3,6-dimethyl-7-(1-(trifluoromethyl)-1H-pyrazol-3-yl)-1H-pyrrolo[3,2-c]pyridin-1-yl)methyl)-3,5-difluorobenzenesulfonamide

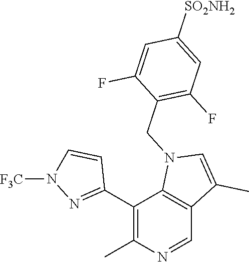 [SMILES: Cc1ncc2c(C)cn(Cc3c(F)cc(S(N)(=O)=O)cc3F)c2c1-c1ccn(C(F)(F)F)n1]

The title compound was synthesized as described in example 14. LCMS: [M+H]⁺=486.1. ¹H NMR: (400 MHZ, DMSO-d₆) δ 8.74 (s, 1H), 8.56 (s, 1H), 7.54 (s, 2H), 7.36 (d, J=6.8 Hz, 2H), 7.00 (s, 1H), 6.69 (s, 1H), 5.01 (s, 2H), 2.29 (s, 6H).

### Example 19: Synthesis of 4-[(6-methoxy-3-methyl-2-oxo-7-phenylimidazo[5,4-c]pyridin-1-yl)methyl]benzene-1-sulfonamide

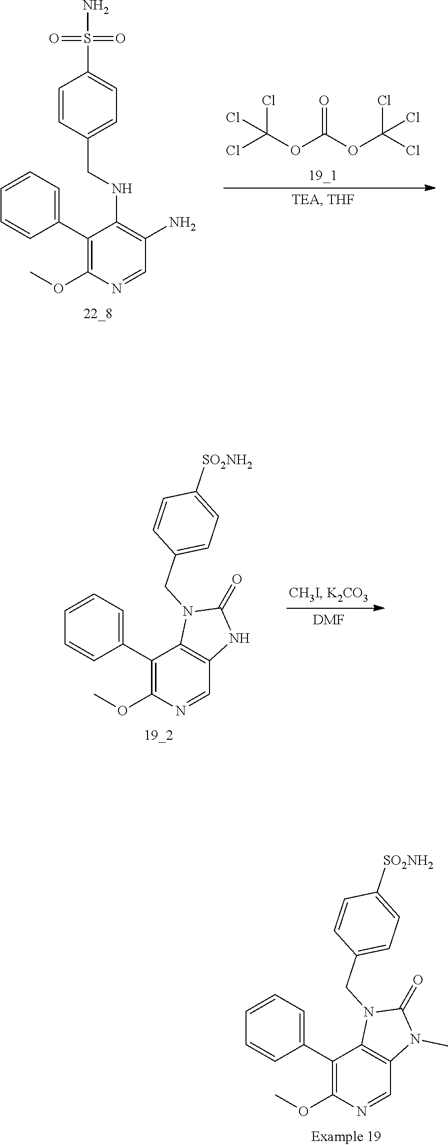

### Step 1: General procedure for preparation of 4-{[(5-amino-2-methoxy-3-phenylpyridin-4-yl)amino]methyl}benzene-1-sulfonamide (Compound 19_2)

To a solution of Compound 22_8 (160.0 mg, 0.42 mmol) in THF (3 mL) were added TEA (126.3 mg, 1.25 mmol) and Compound 19_1 (145.0 mg, 0.83 mmol). The reaction mixture was stirred at 0° C. for 1 h. After the solvent was concentrated under reduced pressure, the obtained residue was purified by flash silica gel chromatography to provide the corresponding Compound 19_2 (120 mg, 70.3% yield) as a yellow solid. LCMS: [M+H]⁺=411.0.

### Step 2: General procedure for preparation of 4-[(6-methoxy-3-methyl-2-oxo-7-phenylimidazo[5,4-c]pyridin-1-yl)methyl]benzene-1-sulfonamide

To a solution of Compound 19_2 (80.0 mg, 0.20 mmol) in DMF (2 mL) were added K₂CO₃ (80.8 mg, 0.59 mmol) and methyl iodide (30.43 mg, 0.214 mmol). The reaction mixture was stirred at room temperature for 6 h. After the solvent was concentrated under reduced pressure, the obtained residue was purified by Prep-HPLC to afford example 19 (15.74 mg, 19.0% yield). LCMS: [M+H]⁺=425.1. ¹H NMR: (400 MHZ, DMSO-d₆) δ 8.02 (s, 1H), 7.54 (d, J=8.4 Hz, 2H), 7.33-7.25 (m, 3H), 7.21 (t, J=7.6 Hz, 2H), 7.01 (d, J=7.2 Hz, 2H), 6.68 (d, J=8.4 Hz, 2H), 4.63 (s, 2H), 3.69 (s, 3H), 3.43 (s, 3H).

### Example 20: Synthesis of 4-((3,6-dimethyl-2-oxo-7-phenyl-2,3-dihydro-1H-imidazo[4,5-c]pyridin-1-yl)methyl)benzenesulfonamide

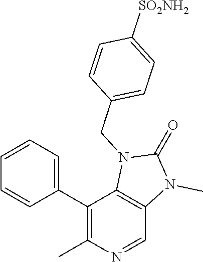 [SMILES: Cc1ncc2c(c1-c1ccccc1)n(Cc1ccc(S(N)(=O)=O)cc1)c(=O)n2C]

The title compound was synthesized as described in example 19. LCMS: [M+H]⁺=409.2. ¹H NMR: (400 MHZ, DMSO-d₆) δ 8.36 (s, 1H), 7.56 (d, J=8.4 Hz, 2H), 7.25 (d, J=7.6 Hz, 5H), 7.01 (d, J=7.2 Hz, 2H), 6.70 (d, J=8.4 Hz, 2H), 4.58 (s, 2H), 3.47 (s, 3H), 2.09 (s, 3H).

### Example 21: Synthesis of 4-((6-methoxy-7-phenyl-1H-pyrazolo[4,3-c]pyridin-1-yl)methyl)benzenesulfonamide

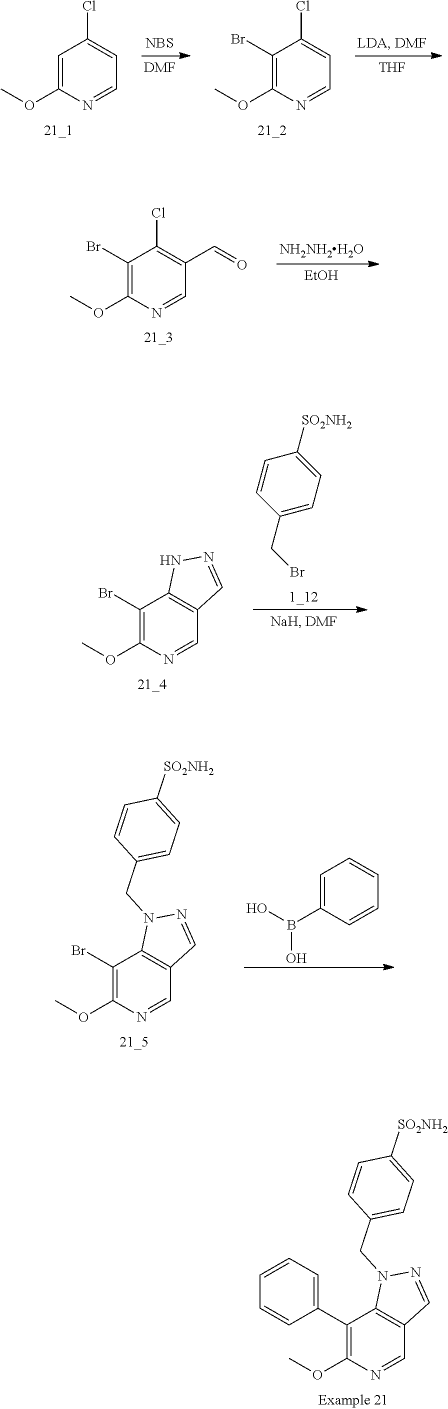

### Step 1: General procedure for preparation of 3-bromo-4-chloro-2-methoxypyridine (Compound 21_2)

To a solution of Compound 21_1 (5.0 g, 34.83 mmol) in DMF (20 mL) was added N-bromosuccinimide (6.2 g, 34.83 mmol). The reaction mixture was stirred at 90° C. for 16 h. After the reaction was quenched with water (30 mL), the resulting aqueous solution was extracted with ethyl acetate (30 mL×3). The combined organic layer was washed with saturated NaCl (30 mL×2), dried over anhydrous Na₂SO₄, and concentrated in vacuum. The obtained residue was purified by flash silica gel chromatography to provide the corresponding Compound 21_2 (2.4 g, 31.0% yield) as a white solid. ¹H NMR: (400 MHz, CDCl₃) § 8.27 (s, 1H), 6.88 (s, 1H), 3.91 (s, 3H).

### Step 2: General Procedure for Preparation of 5-Bromo-4-Chloro-6-Methoxynicotinaldehyde (Compound 21_3)

Under N₂ atmosphere to a solution of Compound 21_2 (1.0 g, 4.5 mmol) in anhydrous THF (20 mL) at −78° C. was slowly added lithium diisopropylamide in n-hexane (2M, 3.4 mL, 6.8 mmol) and the mixture was stirred at −78° C. for 1 h. Anhydrous DMF (1 mL) was added and stirring was continued for 30 min. The mixture was slowly warmed to room temperature and quench by saturated NH₄Cl aq. solution (30 mL). Then the mixture was extracted with ethyl acetate (30 mL×3). The organic phases were combined, washed with water (30 mL×1) and brine (30 mL×1), dried over anhydrous sodium sulfate, filtered, and concentrated under reduced pressure. The obtained residue was purified by flash silica gel chromatography to provide the corresponding Compound 21_3 (0.32 g, yield 28.4%) as a white solid. ¹H NMR: (400 MHZ, CDCl₃) δ 10.39 (s, 1H), 8.46 (s, 1H), 4.06 (s, 3H).

### Step 3: General procedure for preparation of 7-bromo-6-methoxy-1H-pyrazolo[4,3-c]pyridine (Compound 21_4)

To a stirred solution of Compound 21_3 (350 mg, 1.40 mmol) in EtOH(5 mL) was added hydrazine monohydrate (350 mg, 7.0 mmol, 98%). The resulted mixture was stirred at 70° C. overnight in a sealed tube. Water was added to dilute the mixture and then extracted with ethyl acetate (20 mL×3). The combined organic phase was washed with brine (20 mL×1), dried over anhydrous Na₂SO₄, filtered and concentrated under reduced pressure. The obtained residue was purified by flash silica gel chromatography to provide the corresponding Compound 21_4 (130 mg, 40.8%) as a white solid. LCMS: [M+H]⁺=227.9.

### Step 4: General procedure for preparation of 4-((7-bromo-6-methoxy-1H-pyrazolo[4,3-c]pyridin-1-yl)methyl)benzenesulfonamide (Compound 21_4)

To a solution of Compound 21_4 (130 mg, 0.57 mmol) in DMF (5 mL) was added NaH(35 mg, 0.86 mmol, 60% in oil) and the mixture was stirred at 0° C. for 0.5 h under N₂ atmosphere. Compound 1_12 (171 mg, 0.68 mmol) was added to the reaction mixture which was stirred at room temperature for 2 h. After the reaction was completed, the reaction mixture was diluted with water (30 mL). The aqueous phase was extracted with ethyl acetate (20 mL×2). The combined organic phase was washed by brine (20 mL), dried over anhydrous sodium sulfate, filtered, and concentrated in vacuum. The obtained residue was purified by flash silica gel chromatography to provide the corresponding Compound 21_4 (60 mg, 26.5%) as a white solid. LCMS: [M+H]⁺=396.9.

### Step 5: General procedure for preparation of 4-((6-methoxy-7-phenyl-1H-pyrazolo[4,3-c]pyridin-1-yl)methyl)benzenesulfonamide

To a solution of Compound 21_5 (60.0 mg, 0.15 mmol) in dioxane (5 mL) and H₂O (0.5 mL) were added K₂CO₃ (21.8 mg, 0.30 mmol), XPhos-Pd-G2 (40.2 mg, 0.05 mmol) and phenylboronic acid (36.6 mg, 0.30 mmol). The reaction mixture was stirred at 100° C. for 6 h under N₂ atmosphere. After the solvent was concentrated under reduced pressure, the obtained residue was purified by Prep-HPLC to afford example 21 (8.66 mg, 14.5% yield). LCMS: [M+H]⁺=395.2. ¹H NMR: (400 MHz, DMSO-d₆) δ 8.36 (s, 1H), 7.71 (s, 1H), 7.57 (d, J=8.4 Hz, 2H), 7.48-7.36 (m, 3H), 7.34-7.18 (m, 4H), 6.66 (d, J=8.4 Hz, 2H), 5.31 (s, 2H), 4.07 (s, 3H).

### Example 22: Synthesis of 4-((6-methoxy-2-methyl-7-phenyl-1H-imidazo[4,5-c]pyridin-1-yl)methyl)benzenesulfonamide

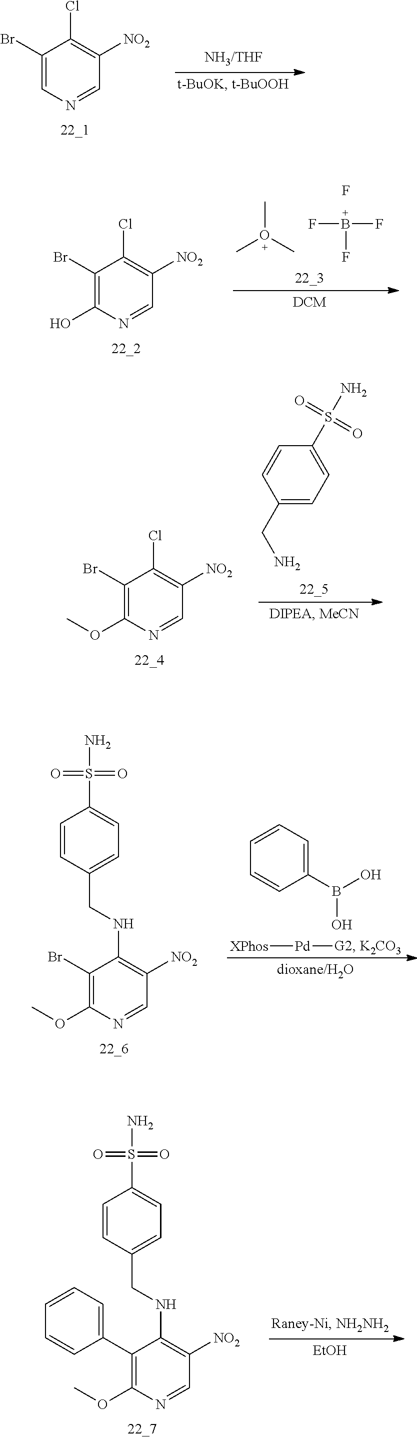

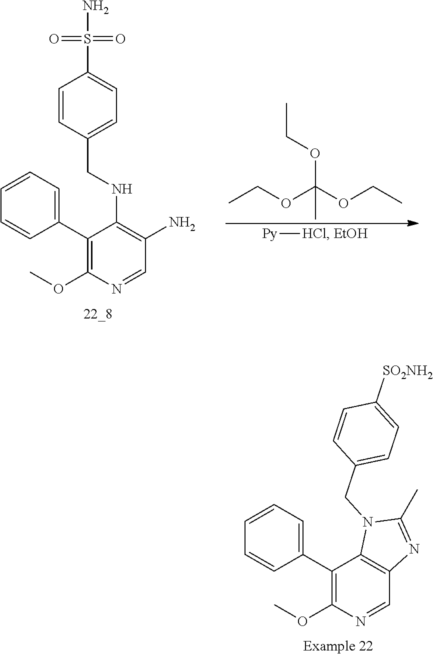

### Step 1: General procedure for preparation of 3-bromo-4-chloro-5-nitropyridin-2-ol (Compound 22_2)

THF (50 mL) was cooled to −78° C. and anhydrous NH₃ (20 mL) was condensed into the THF. Potassium t-butoxide (3.1 g, 27.5 mmol) was added and the mixture was allowed to warm to −35° C. Compound 22_1 (2.6 g, 11 mmol) in THF (20 mL) was cooled to 0° C. and a solution of t-BuOOH(5 M in decane, 2.2 mL, 11 mmol) was added over 5 min. This solution was then added dropwise to the KOt-Bu solution over 0.5 h, then stirred for 1 h at −35° C. and then carefully quenched with 10 mL of sat. NH₄Cl solution. The mixture was warmed to room temperature and stirred overnight. Then the organics were concentrated and the residue was made acidic with NH₄Cl solution and filtered. The solid was washed with cold H₂O and dried to give the title Compound 22_2 (2.5 g, 92%) as a dark brown solid. LCMS: [M−H]⁻=250.9.

### Step 2: General procedure for preparation of 3-bromo-4-chloro-2-methoxy-5-nitropyridine (Compound 22_4)

To a solution of Compound 22_2 (872 mg, 3.45 mmol) in dichloromethane (10 mL) was added Compound 22_3 (1.02 g, 6.89 mmol). The mixture was stirred at 55° C. for 16 h under N₂ atmosphere. After the solvent was concentrated under reduced pressure, the obtained residue was purified by flash silica gel chromatography to provide the corresponding Compound 22_4 (400 mg, 43.5% yield) as a yellow solid. ¹H NMR: (400 MHz, CDCl₃) δ 8.97 (s, 1H), 4.08 (s, 3H).

### Step 3: General procedure for preparation of 4-(((3-bromo-2-methoxy-5-nitropyridin-4-yl)amino) methyl)benzenesulfonamide (Compound 22_6)

To a solution of Compound 22_4 (400 mg, 1.49 mmol) in acetonitrile (5 mL) were added Compound 22_5 (334 mg, 1.49 mmol) and DIPEA (773 mg, 5.99 mmol). The mixture was stirred at 60° C. for 2 h. After the solvent was concentrated under reduced pressure, the obtained residue was purified by flash silica gel chromatography to provide the corresponding Compound 22_6 (288 mg, 46% yield) as a yellow solid. LCMS: [M+H]⁺=416.8, 5.

### Step 4: General procedure for preparation of 4-(((2-methoxy-5-nitro-3-phenylpyridin-4-yl)amino) methyl)benzenesulfonamide (Compound 22_7)

To a solution of Compound 22_6 (288 g, 0.69 mmol) in dioxane-H₂O (5 mL) were added phenylboronic acid (168 mg, 1.38 mmol), XPhos-Pd-G2 (54 mg, 0.068 mmol) and K₂CO₃ (286 mg, 2.07 mmol). The mixture was stirred at 100° C. for 16 h under N₂ atmosphere. After the solvent was concentrated under reduced pressure, the obtained residue was purified by flash silica gel chromatography to provide the corresponding Compound 22_7 (228 g, 79.6% yield) as a green solid. LCMS: [M+H]⁺=415.0, 5.

### Step 5: General procedure for preparation of 4-(((5-amino-2-methoxy-3-phenylpyridin-4-yl)amino) methyl)benzenesulfonamide (Compound 22_8)

To a solution of Compound 22_7 (228 mg, 0.55 mmol) in ethanol (5 mL) were added Raney-Ni (50 mg) and hydrazine monohydrate (138 mg, 2.76 mmol, 98%). The mixture was stirred at room temperature for 1 h. The mixture was filtered, and the filtrate was concentrated under vacuum to give a light yellow solid. The obtained solid was purified by flash silica gel chromatography to provide the corresponding Compound 22_8 (100 mg, 47.3% yield). LCMS: [M+H]⁺=385.1.

### Step 6: General procedure for preparation of 4-((6-methoxy-2-methyl-7-phenyl-1H-imidazo[4,5-c]pyridin-1-yl)methyl)benzenesulfonamide

To a solution of Compound 22_8 (100 mg, 0.26 mmol) in ethanol (3 mL) were added triethyl orthoacetate (422 mg, 2.6 mmol) and Py-HCl (30 mg, 0.26 mmol). The reaction mixture was stirred at 80° C. for 2 h. Solvent was concentrated under reduced pressure and the obtained residue was purified by Prep-HPLC to afford example 22 (73 mg, 68.6% yield). LCMS: [M+H]⁺=409.1. ¹H NMR: (400 MHZ, DMSO-d₆) δ 8.52 (s, 1H), 7.60 (d, J=8.4 Hz, 2H), 7.35-7.27 (m, 3H), 7.22 (t, J=7.6 Hz, 2H), 7.07 (d, J=6.8 Hz, 2H), 6.66 (d, J=8.4 Hz, 2H), 4.93 (s, 2H), 3.76 (s, 3H), 2.37 (s, 3H).

### Example 23: Synthesis of 4-((6-methoxy-7-phenyl-1H-imidazo[4,5-c]pyridin-1-yl)methyl)benzenesulfonamide

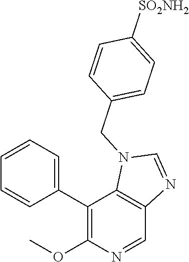 [SMILES: COc1ncc2ncn(Cc3ccc(S(N)(=O)=O)cc3)c2c1-c1ccccc1]

The title compound was synthesized as described in example 22. LCMS: [M+H]⁺=395.1. ¹H NMR: (400 MHZ, DMSO-d₆) δ 8.64 (s, 1H), 8.37 (s, 1H), 7.53 (d, J=8.2 Hz, 2H), 7.35-7.25 (m, 5H), 7.06 (d, J=7.3 Hz, 2H), 6.53 (d, J=8.2 Hz, 2H), 5.07 (s, 2H), 3.77 (s, 3H).

### Example 24: Synthesis of 3,5-difluoro-4-((6-methoxy-2-methyl-7-phenyl-1H-imidazo[4,5-c]pyridin-1-yl)methyl)benzenesulfonamide

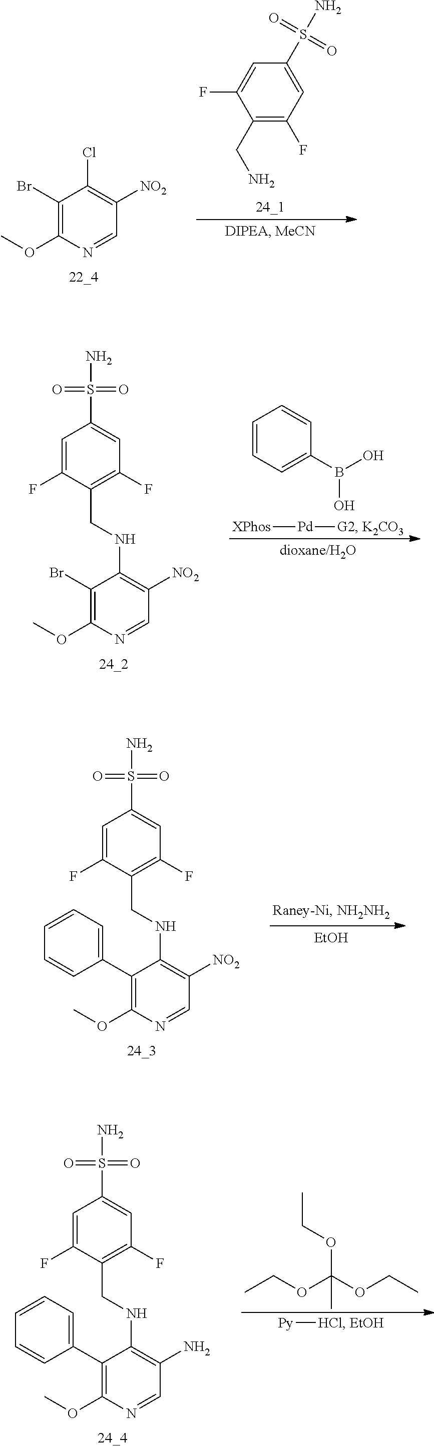

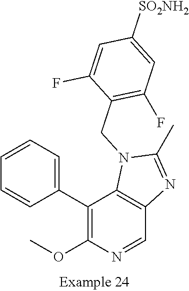 [SMILES: COc1ncc2nc(C)n(Cc3c(F)cc(S(N)(=O)=O)cc3F)c2c1-c1ccccc1]

### Step 1: General procedure for preparation of 4-(((3-bromo-2-methoxy-5-nitropyridin-4-yl)amino) methyl)-3,5-difluorobenzenesulfonamide (Compound 24_3)

To a solution of Compound 22-4 (128 mg, 0.48 mmol) in acetonitrile (3 mL) were added Compound 24_1 (107 mg, 0.48 mmol) and DIPEA (248 mg, 1.93 mmol). The mixture was stirred at 60° C. for 2 h. Solvent was concentrated under reduced pressure and the residue was purified by flash silica gel chromatography to provide the corresponding Compound 24_2 (98 mg, 45.3% yield) as a yellow solid. LCMS: [M+H]⁺=452.8.

### Step 2: General procedure for preparation of 3,5-difluoro-4-(((2-methoxy-5-nitro-3-phenyl pyridin-4-yl)amino) methyl)benzenesulfonamide (Compound 24_3)

To a solution of Compound 24_2 (98 mg, 0.22 mmol) in dioxane-H₂O (3 mL) were added phenylboronic acid (53 mg, 0.44 mmol), XPhos-Pd-G2 (17 mg, 0.02 mmol) and K₂CO₃ (90 mg, 0.065 mmol). The mixture was stirred under 100° C. for 16 h under N₂ atmosphere. Solvent concentrated under reduced pressure and the residue was purified by flash silica gel chromatography to provide the corresponding Compound 24_3 (89 mg, 91.3% yield) as a yellow solid. LCMS: [M+H]⁺=450.9.

### Step 3: General procedure for preparation of 4-(((5-amino-2-methoxy-3-phenylpyridin-4-yl)amino) methyl)-3,5-difluorobenzenesulfonamide (Compound 24_4)

To a solution of Compound 24_3 (89 mg, 0.19 mmol) in ethanol (2 mL) were added Raney-Ni (50 mg) and hydrazine monohydrate (49 mg, 0.98 mmol). The mixture was stirred at room temperature for 1 h. The mixture was filtered and the filtrate was concentrated under vacuum to give a light yellow solid which was purified by flash silica gel chromatography to provide the corresponding Compound 24_4 (28 mg, 33.7% yield) as a yellow solid. LCMS: [M+H]⁺=420.9.

### Step 4: General procedure for preparation of 3,5-difluoro-4-((6-methoxy-2-methyl-7-phenyl-1H-imidazo[4,5-c]pyridin-1-yl)methyl)benzenesulfonamide

To a solution of Compound 24_4 (28 mg, 0.06 mmol) in acetonitrile (2 mL) were added triethyl orthoacetate (108 mg, 0.66 mmol) and Py-HCl (1 mg, 0.006 mmol). The reaction was stirred at 80° C. for 3 h. The reaction mixture was concentrated under reduced pressure and the residue was purified by Prep-HPLC to afford the corresponding example 24 (20 mg, 66.6% yield). LCMS: [M+H]⁺=416.1. ¹H NMR: (400 MHZ, DMSO-d₆) δ 8.46 (s, 1H), 7.61 (s, 2H), 7.35-7.29 (m, 1H), 7.28-7.17 (m, 4H), 7.02 (d, J=7.2 Hz, 2H), 5.12 (s, 2H), 3.74 (s, 3H), 2.48 (s, 3H).

### Example 25: Synthesis of 4-((3,6-dimethyl-2-oxo-7-phenyl-2,3-dihydro-1H-imidazo[4,5-c]pyridin-1-yl)methyl)benzenesulfonamide

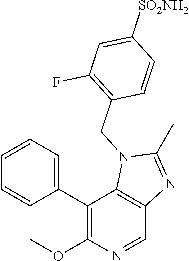 [SMILES: COc1ncc2nc(C)n(Cc3ccc(S(N)(=O)=O)cc3F)c2c1-c1ccccc1]

The title compound was synthesized as described in example 24. LCMS: [M+H]⁺=427.1. ¹H NMR: (400 MHZ, DMSO-d₆) δ 8.53 (s, 1H), 7.52-7.42 (m, 3H), 7.37 (d, J=9.6 Hz, 1H), 7.30 (t, J=7.6 Hz, 1H), 7.17 (t, J=7.6 Hz, 2H), 6.94 (d, J=7.2 Hz, 2H), 6.42 (t, J=7.6 Hz, 1H), 4.97 (s, 2H), 3.75 (s, 3H), 2.44 (s, 3H).

### Example 26: Synthesis of 4-((2,6-dimethyl-7-phenyl-1H-imidazo[4,5-c]pyridin-1-yl)methyl)-3,5-difluorobenzenesulfonamide

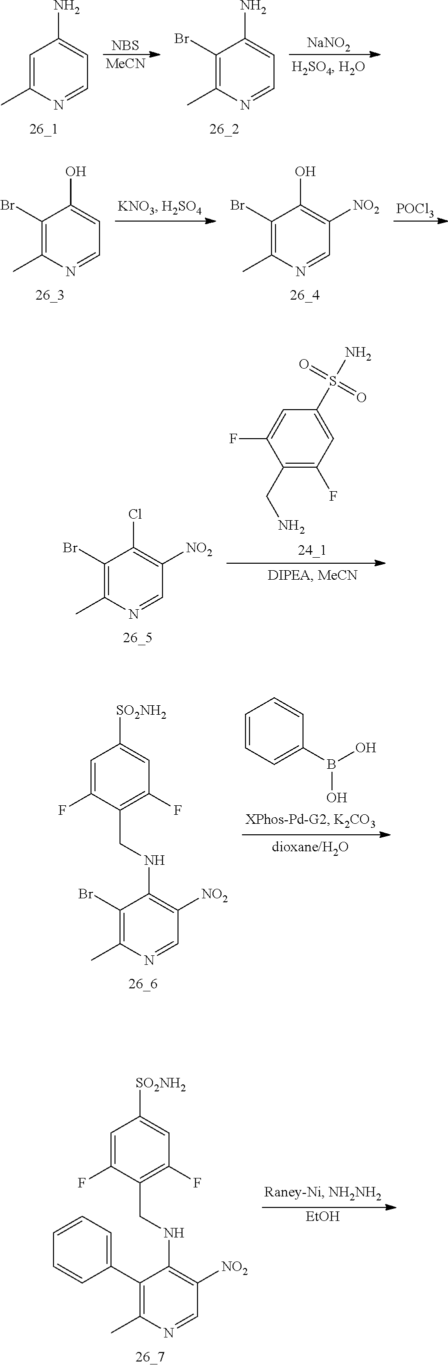

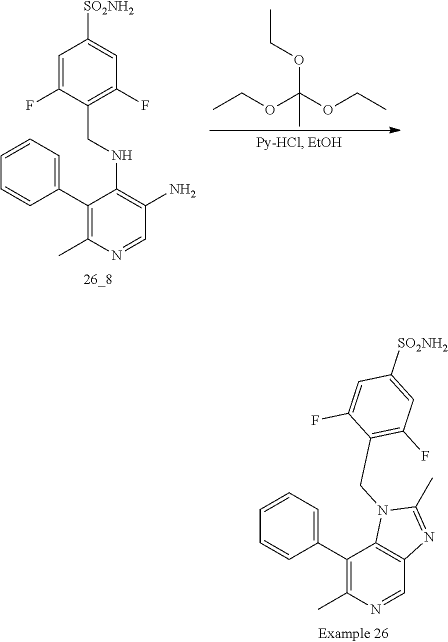 [SMILES: Cc1ncc(N)c(NCc2c(F)cc(S(N)(=O)=O)cc2F)c1-c1ccccc1 ; CCOC(C)(OCC)OCC ; Cc1ncc2nc(C)n(Cc3c(F)cc(S(N)(=O)=O)cc3F)c2c1-c1ccccc1]

### Step 1: General procedure for preparation of 3-bromo-2-methylpyridin-4-amine (Compound 26_2)

To a solution of Compound 26_1 (7.6 g, 70.4 mmol) in acetonitrile (80 mL) was added NBS (37 g, 207.8 mmol) at −50° C. The mixture was stirred at −50° C. for 3 h. Solvent was concentrated under reduced pressure and the residue was purified by flash silica gel chromatography to provide the corresponding Compound 26_2 (7.4 g, 56% yield) as a white solid. ¹H NMR: (400 MHZ, DMSO-d₆) δ 7.82 (d, J=5.6 Hz, 1H), 6.51 (d, J=5.6 Hz, 1H), 6.15 (s, 2H), 2.42 (s, 3H).

### Step 2: General procedure for preparation of 3-bromo-2-methylpyridin-4-ol (Compound 26_3)

To a solution of Compound 26_2 (7.4 g, 39.8 mmol) in H₂O (60 mL) and H₂SO₄ (30 mL) was added NaNO₂ (5.5 g, 79.6 mmol) at 0° C. The mixture was stirred at room temperature for 3 h. After heated at 100° C. for 6 h, solvent was removed under reduced pressure and the residue was purified by flash silica gel chromatography to provide the corresponding Compound 26_3 (3.8 g, 51% yield) as a white solid.

### Step 3: General procedure for preparation of 3-bromo-2-methyl-5-nitropyridin-4-ol (Compound 26_4)

To a solution of Compound 26_3 (3.8 g, 20.3 mmol) in H₂SO₄ (20 mL) was added KNO₃ (4.02 g, 40.6 mmol). The mixture was stirred at 80° C. for 16 h. After the reaction mixture was cooled to 0° C., ammonium hydroxide was carefully added to adjust pH to about 5˜6. The title compound precipitated and was collected by filtration. The obtained solid was then dried in vacuo at 50° C. to give Compound 26_4 (2.65 g, 48.3% yield) as a pale yellow solid. LCMS: [M+H]⁺=232.9.

### Step 4: General procedure for preparation of 3-bromo-4-chloro-2-methyl-5-nitropyridine (Compound 26_5)

To a solution of Compound 26_4 (2.65 g, 11.4 mmol) in POCl₃ (20 mL) was added N,N-dimethylaniline (1.38 g, 11.4 mmol) at 0° C. This reaction mixture was stirred at 100° C. for 1 h. Solvent was removed in vacuum to give crude product which was purified by flash silica gel chromatography to afford Compound 26_5 (1.46 g, 51.1% yield) as a yellow solid. LCMS: [M+H]⁺=251.0.

### Step 5: General procedure for preparation of 4-(((3-bromo-2-methyl-5-nitropyridin-4-yl)amino) methyl)-3,5-difluorobenzenesulfonamide (Compound 26_6)

To a solution of Compound 26_5 (1.46 g, 5.84 mmol) in acetonitrile (12 mL) were added Compound 24_2 (1.30 g, 5.84 mmol) and DIPEA (2.63 g, 11.68 mmol). This reaction mixture was stirred at 60° C. for 2 h. Solvent was removed under reduced pressure and the residue was purified by flash silica gel chromatography to provide the corresponding Compound 26_6 (624 mg, 24.7% yield) as a white solid. LCMS: [M+H]⁺=437.0.

### Step 6: General procedure for preparation of 3,5-difluoro-4-(((2-methyl-5-nitro-3-phenyl pyridin-4-yl)amino) methyl)benzenesulfonamide (Compound 26_7)

To a solution of Compound 26_6 (624 mg, 1.43 mmol) in dioxane-H₂O (10 mL/1 mL) were added phenylboronic acid (349 mg, 2.86 mmol), XPhos-Pd-G2 (60 mg, 0.14 mmol), Pd₂ (dba) 3 (131 mg, 0.14 mmol) and K₂CO₃ (624 mg, 4.29 mmol). This reaction mixture was stirred at 100° C. for 16 h under N₂ atmosphere. Solvent was removed under reduced pressure and the residue was purified by flash silica gel chromatography to provide the corresponding Compound 26_9 (372 mg, 0.16 mmol, 42.7% yield) as a white solid. LCMS: [M+H]⁺=435.3.

### Step 7: General procedure for preparation of 4-(((5-amino-2-methyl-3-phenylpyridin-4-yl)amino) methyl)-3,5-difluorobenzenesulfonamide (Compound 26_8)

To a solution of Compound 26_7 (372 mg, 0.85 mmol) in ethanol (5 mL) were added Raney-Ni (100 mg, 1.6 mmol) and hydrazine monohydrate (214 mg, 4.25 mmol). This reaction mixture was stirred at room temperature for 10 min. The mixture was filtered and the filtrate was concentrated in vacuum to give a light yellow solid which was purified by flash silica gel chromatography to provide the corresponding Compound 26_8 (173 mg, 50% yield) as a white solid. LCMS: [M+H]⁺=404.9.

### Step 8: General procedure for preparation of 4-((2,6-dimethyl-7-phenyl-1H-imidazo[4,5-c]pyridin-1-yl)methyl)-3,5-difluorobenzenesulfonamide

To a solution of Compound 26_8 (173 mg, 0.43 mmol) in ethanol (3 mL) were added Compound 24_7 (693 mg, 4.3 mmol) and Py-HCl (49 mg, 0.43 mmol). The reaction mixture was stirred at 80° C. for 2 h. Solvent was removed under reduced pressure and the residue was purified by Prep-HPLC to afford the corresponding example 26 (40 mg, 0.12 mmol, 21.8% yield). LCMS: [M+H]⁺=429.1. ¹H NMR: (400 MHZ, DMSO-d₆) δ 8.73 (s, 1H), 7.62 (s, 2H), 7.40-7.30 (m, 1H), 7.33-7.21 (m, 4H), 7.07-7.02 (m, 2H), 5.09 (s, 2H), 2.49 (s, 3H), 2.16 (s, 3H).

### Example 27: Synthesis of 3,5-difluoro-4-((6-methyl-7-phenyl-1H-imidazo[4,5-c]pyridin-1-yl)methyl)benzene sulfonamide

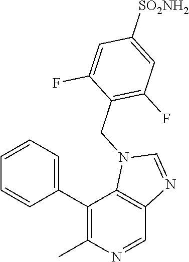 [SMILES: Cc1ncc2ncn(Cc3c(F)cc(S(N)(=O)=O)cc3F)c2c1-c1ccccc1]

The title compound was synthesized as described in example 26. LCMS: [M+H]⁺=415.1. ¹H NMR: (400 MHZ, DMSO-d₆) δ 8.87 (s, 1H), 8.30 (s, 1H), 7.63 (s, 2H), 7.38 (t, J=7.4 Hz, 1H), 7.32 (t, J=7.3 Hz, 2H), 7.25 (d, J=7.4 Hz, 2H), 7.11 (d, J=7.0 Hz, 2H), 5.17 (s, 2H), 2.20 (s, 3H).

### Example 28: Synthesis of 3,5-difluoro-4-((6-methyl-7-(o-tolyl)-1H-imidazo[4,5-c]pyridin-1-yl)methyl)benzene sulfonamide

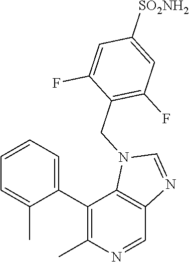 [SMILES: Cc1ccccc1-c1c(C)ncc2ncn(Cc3c(F)cc(S(N)(=O)=O)cc3F)c12]

The title compound was synthesized as described in example 26. LCMS: [M+H]⁺=429.1. ¹H NMR: (400 MHZ, DMSO-d₆) δ 8.87 (s, 1H), 8.31 (s, 1H), 7.65 (s, 2H), 7.36-7.22 (m, 3H), 7.22-7.11 (m, 2H), 7.03 (d, J=6.8 Hz, 1H), 5.16-5.06 (m, 2H), 2.12 (s, 3H), 1.69 (s, 3H).

### Example 29: Synthesis of 4-((6-methyl-7-phenyl-1H-imidazo[4,5-c]pyridin-1-yl)methyl)benzene sulfonamide

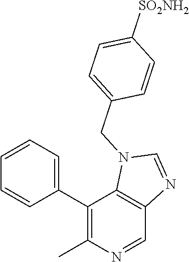 [SMILES: Cc1ncc2ncn(Cc3ccc(S(N)(=O)=O)cc3)c2c1-c1ccccc1]

The title compound was synthesized as described in example 26. LCMS: [M+H]⁺=379.1. ¹H NMR: (400 MHZ, DMSO-d₆) δ 8.90 (s, 1H), 8.42 (s, 1H), 7.56 (d, J=8.4 Hz, 2H), 7.38 (t, J=7.6 Hz, 1H), 7.33-7.23 (m, 4H), 7.12-C_{6.98} (m, 2H), 6.55 (d, J=8.4 Hz, 2H), 5.04 (s, 2H), 2.21 (s, 3H).

### Example 30: Synthesis of 4-((4-methyl-7-phenyl-1H-imidazo[4,5-c]pyridin-1-yl)methyl)benzene sulfonamide

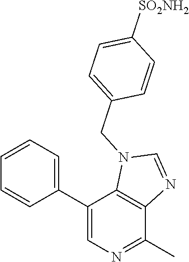 [SMILES: Cc1ncc(-c2ccccc2)c2c1ncn2Cc1ccc(S(N)(=O)=O)cc1]

The title compound was synthesized as described in example 26. LCMS: [M+H]⁺=379.1. ¹H NMR: (400 MHZ, CD₃OD) δ 8.49 (s, 1H), 7.95 (s, 1H), 7.58 (d, J=8.4 Hz, 2H), 7.42 (t, J=7.4 Hz, 1H), 7.34 (t, J=7.4 Hz, 2H), 7.20-7.07 (m, 2H), 6.52 (d, J=8.5 Hz, 2H), 5.36 (s, 2H), 2.89 (s, 3H).

### Example 31: Synthesis of 4-((2,6-dimethyl-7-phenyl-1H-imidazo[4,5-c]pyridin-1-yl)methyl)benzene sulfonamide

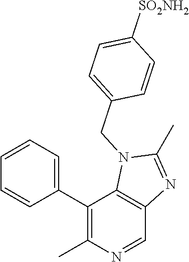 [SMILES: Cc1ncc2nc(C)n(Cc3ccc(S(N)(=O)=O)cc3)c2c1-c1ccccc1]

The title compound was synthesized as described in example 26. LCMS: [M+H]⁺=393.1. ¹H NMR: (400 MHZ, DMSO-d₆) δ 8.79 (s, 1H), 7.62 (d, J=8.4 Hz, 2H), 7.38-7.25 (m, 5H), 7.08 (d, J=7.2 Hz, 2H), 6.66 (d, J=8.4 Hz, 2H), 4.90 (s, 2H), 2.38 (s, 3H), 2.19 (s, 3H).

### Example 32: Synthesis of 4-((2,4-dimethyl-7-phenyl-1H-imidazo[4,5-c]pyridin-1-yl)methyl)benzene sulfonamide

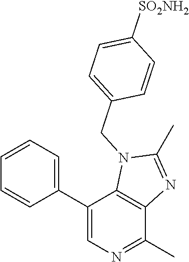 [SMILES: Cc1ncc(-c2ccccc2)c2c1nc(C)n2Cc1ccc(S(N)(=O)=O)cc1]

The title compound was synthesized as described in example 26. LCMS: [M+H]⁺=393.1. ¹H NMR: (400 MHZ, DMSO-d₆) δ 7.89 (s, 1H), 7.58 (d, J=8.3 Hz, 2H), 7.38 (t, J=7.4 Hz, 1H), 7.29 (t, J=7.4 Hz, 4H), 7.17 (d, J=7.1 Hz, 2H), 6.64 (d, J=8.2 Hz, 2H), 5.16 (s, 2H), 2.75 (s, 3H), 2.48 (s, 3H).

### Example 33: Synthesis of 4-((6-(difluoromethoxy)-2-methyl-7-phenyl-1H-imidazo[4,5-c]pyridin-1-yl)methyl)benzenesulfonamide

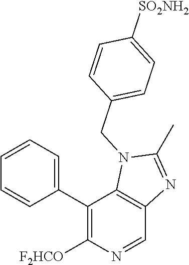 [SMILES: Cc1nc2cnc(OC(F)F)c(-c3ccccc3)c2n1Cc1ccc(S(N)(=O)=O)cc1]

The title compound was synthesized as described in example 22. LCMS: [M+H]⁺=445.1. ¹H NMR: (400 MHZ, DMSO-d₆) δ 8.61 (s, 1H), 7.81-7.44 (t, J=72.0 Hz, 1H), 7.60 (d, J=8.3 Hz, 2H), 7.36 (d, J=7.5 Hz, 1H), 7.32 (s, 2H), 7.27 (t, J=7.6 Hz, 2H), 7.12 (d, J=7.2 Hz, 2H), 6.67 (d, J=8.3 Hz, 2H), 5.00 (s, 2H), 2.43 (s, 3H).

### Example 34: Synthesis of 3,5-difluoro-4-((6-methyl-7-phenyl-2-(trifluoromethyl)-1H-imidazo[4,5-c]pyridin-1-yl)methyl)benzenesulfonamide

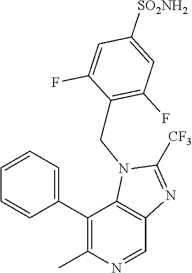 [SMILES: Cc1ncc2nc(C(F)(F)F)n(Cc3c(F)cc(S(N)(=O)=O)cc3F)c2c1-c1ccccc1]

The title compound was synthesized as described in example 26. LCMS: [M+H]⁺=483.1. ¹H NMR: (400 MHZ, DMSO-d₆) δ 9.12 (s, 1H), 7.65 (s, 2H), 7.40 (t, J=7.5 Hz, 1H), 7.32-7.26 (m, 4H), 7.10 (d, J=7.3 Hz, 2H), 5.29 (s, 2H), 2.23 (s, 3H).

### Example 35: Synthesis of 4-(1-(6-methoxy-2-methyl-7-phenyl-1H-imidazo[4,5-c]pyridin-1-yl)ethyl) benzenesulfonamide

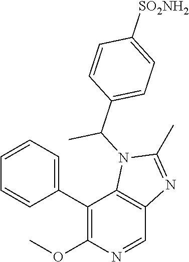 [SMILES: COc1ncc2nc(C)n(C(C)c3ccc(S(N)(=O)=O)cc3)c2c1-c1ccccc1]

The title compound was synthesized as described in example 22. LCMS: [M+H]⁺=423.4. ¹H NMR: (400 MHZ, DMSO-d₆) δ 8.48 (s, 1H), 7.70 (d, J=8.0 Hz, 2H), 7.50-7.29 (m, 7H), 7.10 (d, J=8.0 Hz, 2H), 5.16-5.04 (m, 1H), 3.77 (s, 3H), 1.97 (s, 3H), 1.56 (d, J=6.8 Hz, 3H).

### Example 36: Synthesis of 2-fluoro-4-((6-methoxy-2-methyl-7-phenyl-1H-imidazo[4,5-c]pyridin-1-yl)methyl)benzenesulfonamide

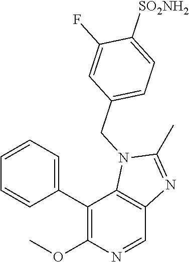 [SMILES: COc1ncc2nc(C)n(Cc3ccc(S(N)(=O)=O)c(F)c3)c2c1-c1ccccc1]

The title compound was synthesized as described in example 24. LCMS: [M+H]⁺=427.2.

¹H NMR: (400 MHZ, CD₃OD) δ 8.47 (s, 1H), 7.62 (t, J=8.0 Hz, 1H), 7.31 (t, J=7.2 Hz, 1H), 7.21 (t, J=7.6 Hz, 2H), 6.99 (d, J=6.8 Hz, 2H), 6.37-C_{6.30} (m, 2H), 5.03 (s, 2H), 3.80 (s, 3H), 2.49 (s, 3H).

### Example 37: Synthesis of 4-((6-methoxy-2-methyl-7-phenyl-1H-imidazo[4,5-c]pyridin-1-yl)methyl)phenyl sulfamate

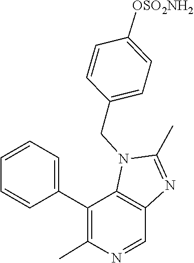

The title compound was synthesized as described in example 26. LCMS: [M+H]⁺=409.0.

¹H NMR: (400 MHZ, DMSO-d₆) δ 8.79 (s, 1H), 7.96 (s, 2H), 7.40-7.25 (m, 3H), 7.13-7.05 (m, 4H), 6.54 (d, J=8.7 Hz, 2H), 4.84 (s, 2H), 2.40 (s, 3H), 2.20 (s, 3H).

### Example 38: Synthesis of 4-((2-(difluoromethyl)-6-methyl-7-phenyl-1H-imidazo[4,5-c]pyridin-1-yl)methyl)-3,5-difluorobenzenesulfonamide

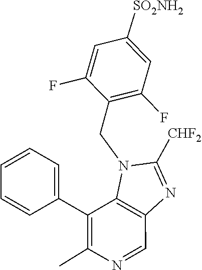 [SMILES: Cc1ncc2nc(C(F)F)n(Cc3c(F)cc(S(N)(=O)=O)cc3F)c2c1-c1ccccc1]

The title compound was synthesized as described in example 26. LCMS: [M+H]⁺=465.1. ¹H NMR: (400 MHZ, DMSO-d₆) δ 9.03 (s, 1H), 7.64 (s, 2H), 7.48 (t, J=51.6 Hz, 1H), 7.37 (t, J=7.6 Hz, 1H), 7.31-7.20 (m, 4H), 7.09 (d, J=7.6 Hz, 2H), 5.27 (s, 2H), 2.21 (s, 3H).

### Example 39: Synthesis of 4-((2-cyclopropyl-6-methyl-7-phenyl-1H-imidazo[4,5-c]pyridin-1-yl)methyl)-3,5-difluorobenzenesulfonamide

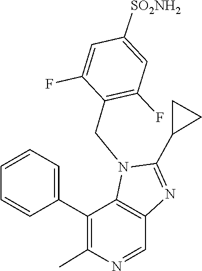 [SMILES: Cc1ncc2nc(C3CC3)n(Cc3c(F)cc(S(N)(=O)=O)cc3F)c2c1-c1ccccc1]

The title compound was synthesized as described in example 26. LCMS: [M+H]⁺=455.1. ¹H NMR: (400 MHZ, DMSO-d₆) § 8.70 (s, 1H), 7.63 (s, 2H), 7.40-7.24 (m, 5H), 7.09 (d, J=7.6 Hz, 2H), 5.22 (s, 2H), 2.22-7.24 (m, 1H), 2.16 (s, 3H), 1.04-0.91 (m, 4H).

### Example 40: Synthesis of 4-((2-methyl-7-phenyl-1H-imidazo[4,5-c]pyridin-1-yl)methyl)benzene sulfonamide

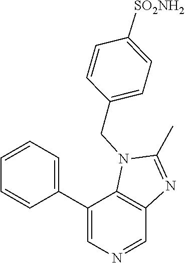 [SMILES: Cc1nc2cncc(-c3ccccc3)c2n1Cc1ccc(S(N)(=O)=O)cc1]

The title compound was synthesized as described in example 22. LCMS: [M+H]⁺=379.1. ¹H NMR: (400 MHZ, DMSO-d₆) δ 8.91 (s, 1H), 8.05 (s, 1H), 7.57 (d, J=8.4 Hz, 2H), 7.40 (t, J=7.4 Hz, 1H), 7.32 (t, J=7.5 Hz, 2H), 7.21 (d, J=6.9 Hz, 2H), 7.12 (bs, 2H), 6.64 (d, J=8.3 Hz, 2H), 5.17 (s, 2H), 2.49 (s, 3H).

### Example 41: Synthesis of 4-((6-cyano-2-methyl-7-phenyl-1H-imidazo[4,5-c]pyridin-1-yl)methyl)benzenesulfonamide

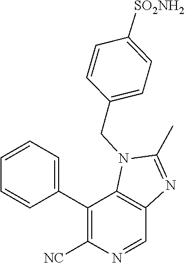 [SMILES: Cc1nc2cnc(C#N)c(-c3ccccc3)c2n1Cc1ccc(S(N)(=O)=O)cc1]

The title compound was synthesized as described in example 22. LCMS: [M+H]⁺=404.1. ¹H NMR: (400 MHZ, DMSO-d₆) δ 8.23 (s, 1H), 7.56 (d, J=8.4 Hz, 2H), 7.44 (t, J=7.5 Hz, 1H), 7.36-7.30 (m, 4H), 7.24 (d, J=7.0 Hz, 2H), 6.68 (d, J=8.4 Hz, 2H), 5.23 (s, 2H), 2.60 (s, 3H).

### Example 42: Synthesis of 3,5-difluoro-4-((6-methyl-2-(methyl-d₃)-7-phenyl-1H-imidazo[4,5-c]pyridin-1-yl)methyl)benzenesulfonamide

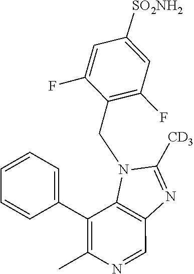 [SMILES: [2H]C([2H])([2H])c1nc2cnc(C)c(-c3ccccc3)c2n1Cc1c(F)cc(S(N)(=O)=O)cc1F]

The title compound was synthesized as described in example 26. LCMS: [M+H]⁺=432.1. ¹H NMR: (400 MHZ, DMSO-d₆) δ 8.73 (s, 1H), 7.63 (s, 2H), 7.38 (t, J=7.4 Hz, 1H), 7.34-7.19 (m, 4H), 7.04 (d, J=7.2 Hz, 2H), 5.09 (s, 2H), 2.15 (s, 3H).

### Example 43: Synthesis of 3,5-difluoro-4-((2-methyl-7-phenyl-1H-imidazo[4,5-c]pyridin-1-yl)methyl)benzenesulfonamide

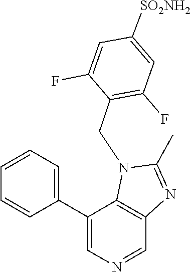 [SMILES: Cc1nc2cncc(-c3ccccc3)c2n1Cc1c(F)cc(S(N)(=O)=O)cc1F]

The title compound was synthesized as described in example 26. LCMS: [M+H]⁺=415.1.

¹H NMR: (400 MHZ, DMSO-d₆) δ 8.86 (s, 1H), 7.99 (s, 1H), 7.61 (s, 2H), 7.40 (t, J=7.4 Hz, 1H), 7.34 (t, J=7.2 Hz, 2H), 7.22-7.12 (m, 4H), 5.36 (s, 2H), 2.59 (s, 3H).

### Example 44: Synthesis of 3,5-difluoro-4-((7-(2-fluorophenyl)-2,6-dimethyl-1H-imidazo[4,5-c]pyridin-1-yl)methyl)benzenesulfonamide

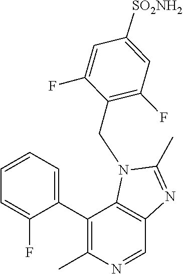 [SMILES: Cc1ncc2nc(C)n(Cc3c(F)cc(S(N)(=O)=O)cc3F)c2c1-c1ccccc1F]

The title compound was synthesized as described in example 26. LCMS: [M+H]⁺=447.2. ¹H NMR: (400 MHZ, DMSO-d₆) δ 8.77 (s, 1H), 7.64 (s, 2H), 7.49-7.42 (m, 1H), 7.26-7.16 (m, 4H), 7.10 (t, J=9.1 Hz, 1H), 5.23-5.12 (m, 2H), 2.55 (s, 3H), 2.15 (s, 3H).

### Example 45: Synthesis of 3,5-difluoro-4-((7-(3-fluorophenyl)-2,6-dimethyl-1H-imidazo[4,5-c]pyridin-1-yl)methyl)benzenesulfonamide

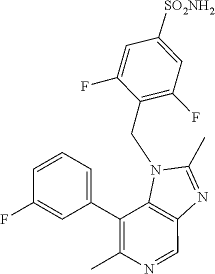 [SMILES: Cc1ncc2nc(C)n(Cc3c(F)cc(S(N)(=O)=O)cc3F)c2c1-c1cccc(F)c1]

The title compound was synthesized as described in example 26. LCMS: [M+H]⁺=447.2. ¹H NMR: (400 MHz, DMSO-d₆) § 8.75 (s, 1H), 7.61 (s, 2H), 7.32-7.18 (m, 4H), 7.07-C_{6.98} (m, 1H), 6.82-C_{6.77} (m, 1H), 5.23-5.06 (m, 2H), 2.52 (s, 3H), 2.17 (s, 3H).

### Example 46: Synthesis of 3,5-difluoro-4-((7-(4-fluorophenyl)-2,6-dimethyl-1H-imidazo[4,5-c]pyridin-1-yl)methyl)benzenesulfonamide

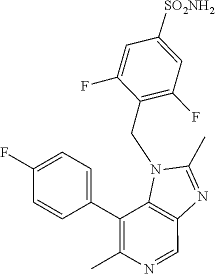 [SMILES: Cc1ncc2nc(C)n(Cc3c(F)cc(S(N)(=O)=O)cc3F)c2c1-c1ccc(F)cc1]

The title compound was synthesized as described in example 26. LCMS: [M+H]⁺=447.2. ¹H NMR: (400 MHZ, DMSO-d₆) δ 8.73 (s, 1H), 7.63 (s, 2H), 7.27 (d, J=8.0 Hz, 2H), 7.16-7.02 (m, 4H), 5.13 (s, 2H), 2.51 (s, 3H), 2.14 (s, 3H).

### Example 47: Synthesis of 3,5-difluoro-4-((2-(fluoromethyl)-6-methyl-7-phenyl-1H-imidazo[4,5-c]pyridin-1-yl)methyl)benzenesulfonamide

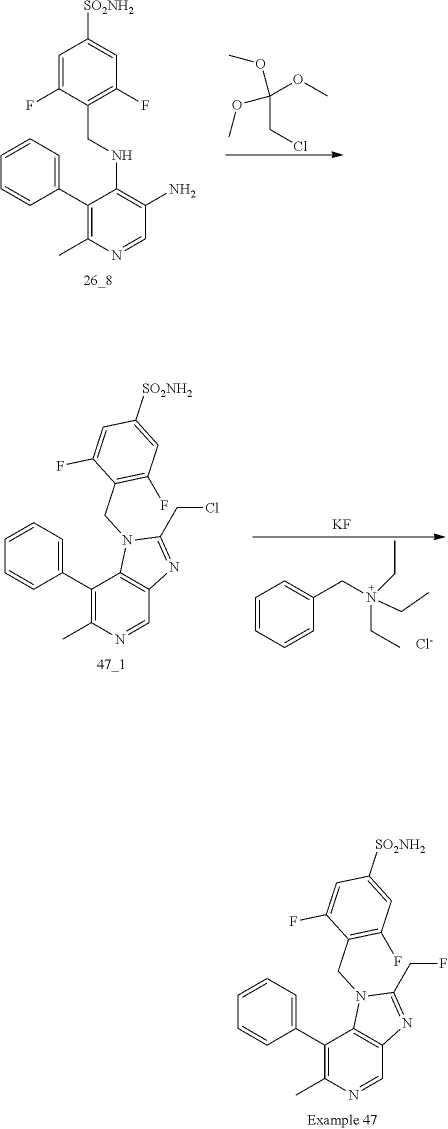

### Step 1: General procedure for preparation of 4-((2-(chloromethyl)-6-methyl-7-phenyl-1H-imidazo[4,5-c]pyridin-1-yl)methyl)-3,5-difluorobenzenesulfonamide (Compound 47_1)

To a solution of Compound 26_8 (200 mg, 0.495 mmol) in EtOH(1 mL) were added 2-chloro-1,1,1-trimethoxyethane (1 mL) and p-Toluenesulfonic acid monohydrate (47.03 mg, 0.247 mmol). The reaction mixture was irradiated with microwave radiation at 120° C. for 1 h. The reaction was quenched by H₂O dropwise at room temperature. The aqueous layer was extracted with ethyl acetate (15 mL×3). The combined organic layers were washed with brine, dried over anhydrous Na₂SO₄, filtered and concentrated under reduced pressure. The residue was purified by flash silica gel chromatography to provide the corresponding Compound 47_1 (129 mg, 56.35%) as a white solid. LCMS: [M+H]⁺=463.1. Step 2: General procedure for preparation of 3,5-difluoro-4-((2-(fluoromethyl)-6-methyl-7-phenyl-1H-imidazo[4,5-c]pyridin-1-yl)methyl)benzenesulfonamide

To a solution of Compound 47_1 (55 mg, 0.119 mmol) in DMSO (1 mL) were added benzyltriethylammonium chloride (2.71 mg, 0.012 mmol) and KF (20.71 mg, 0.356 mmol). The reaction mixture was stirred at 70° C. for 16 h. The reaction mixture was concentrated under reduced pressure and the obtained residue was purified by Prep-HPLC to afford the corresponding example 47 (2.1 mg, 3.96%). LCMS: [M+H]⁺=447.1. ¹H NMR: (400 MHZ, CD₃OD) δ 8.88 (s, 1H), 7.42 (t, J=7.2 Hz, 1H), 7.33 (t, J=7.7 Hz, 2H), 7.26 (d, J=7.6 Hz, 2H), 7.11 (d, J=7.3 Hz, 2H), 5.69 (d, J=47.2 Hz, 2H), 5.30 (s, 2H), 2.28 (s, 3H).

### Example 48: Synthesis of 4-((6-ethyl-2-methyl-7-phenyl-1H-imidazo[4,5-c]pyridin-1-yl)methyl)-3,5-difluorobenzenesulfonamide

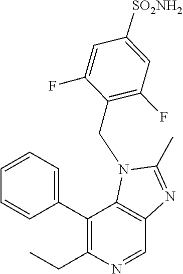 [SMILES: CCc1ncc2nc(C)n(Cc3c(F)cc(S(N)(=O)=O)cc3F)c2c1-c1ccccc1]

The title compound was synthesized as described in example 26. LCMS: [M+H]⁺=443.2. ¹H NMR: (400 MHZ, DMSO-d₆) δ 8.79 (s, 1H), 7.65 (s, 2H), 7.41-7.33 (m, 1H), 7.30-7.22 (m, 4H), 7.05 (d, J=7.2 Hz, 2H), 5.07 (s, 2H), 2.51 (s, 3H), 2.40 (q, J=7.6 Hz, 2H), 1.02 (t, J=7.6 Hz, 3H).

### Example 49: Synthesis of 4-((6-ethyl-2-methyl-7-phenyl-1H-imidazo[4,5-c]pyridin-1-yl)methyl)-3,5-difluorobenzenesulfonamide

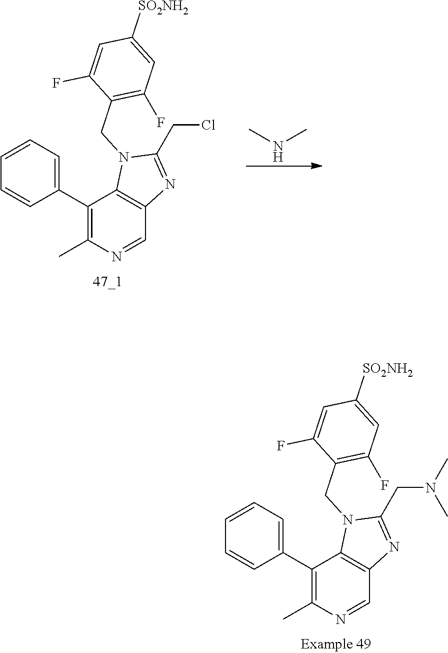

To a solution of Compound 47_1 (60 mg, 0.13 mmol) in dimethylamine/THF (3 mL, 2M, 6.0 mmol) was added DIPEA (0.043 mL, 0.26 mmol). The reaction mixture was stirred at 60° C. for 1 h. The reaction mixture was concentrated under reduced pressure and the obtained residue was purified by Prep-HPLC to afford the corresponding example 49 (38 mg, 64.1%). LCMS: [M+H]⁺=472.1. ¹H NMR (400 MHz, DMSO-d₆) δ 8.81 (s, 1H), 7.35 (t, J=7.3 Hz, 1H), 7.28 (t, J=7.3 Hz, 2H), 7.15-7.05 (m, 4H), 6.25 (brs, 2H), 5.18 (s, 2H), 3.62 (s, 2H), 2.18 (s, 3H), 2.11 (s, 6H).

### Example 50: Synthesis of 3,5-difluoro-4-((6-methyl-7-phenyl-2-(2,2,2-trifluoroethyl)-1H-imidazo[4,5-c]pyridin-1-yl)methyl)benzenesulfonamide

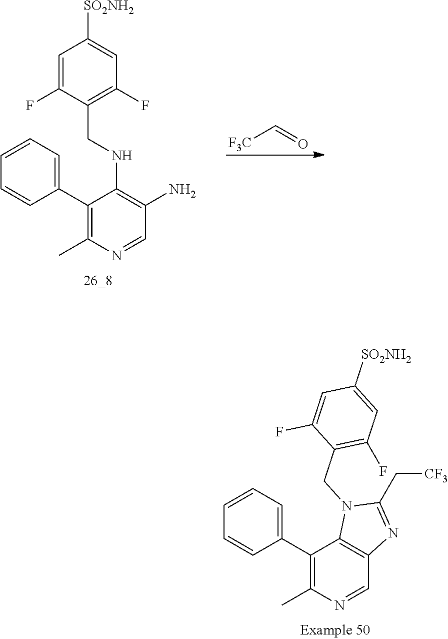 [SMILES: Cc1ncc(N)c(NCc2c(F)cc(S(N)(=O)=O)cc2F)c1-c1ccccc1 ; O=CC(F)(F)F ; Cc1ncc2nc(CC(F)(F)F)n(Cc3c(F)cc(S(N)(=O)=O)cc3F)c2c1-c1ccccc1]

To a solution of Compound 26_8 (100 mg, 0.247 mmol) in EtOH(5 mL) was added 2,2,2-trifluoroacetaldehyde (121 mg, 1.235 mmol). The reaction mixture was stirred at 70° C. for 16 h. The solvent was removed under reduced pressure and the obtained residue was purified by Prep-HPLC to afford the corresponding example 50 (0.87 mg, 0.71%). LCMS: [M+H]⁺=497.2.

### Example 51: Synthesis of 4-((2-(cyclopropylmethyl)-6-methyl-7-phenyl-1H-imidazo[4,5-c]pyridin-1-yl)methyl)-3,5-difluorobenzenesulfonamide

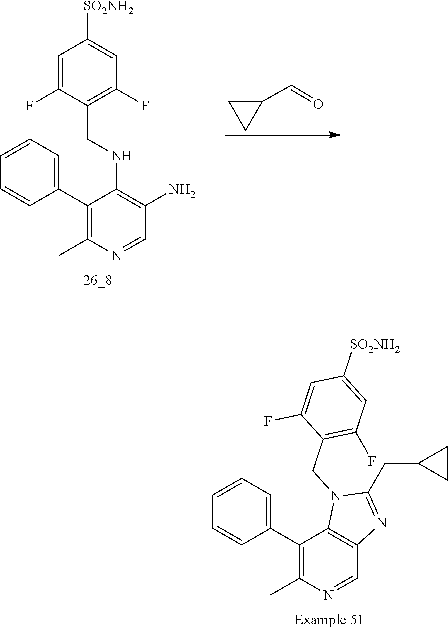 [SMILES: Cc1ncc(N)c(NCc2c(F)cc(S(N)(=O)=O)cc2F)c1-c1ccccc1 ; O=CC1CC1 ; Cc1ncc2nc(CC3CC3)n(Cc3c(F)cc(S(N)(=O)=O)cc3F)c2c1-c1ccccc1]

To a solution of Compound 26_8 (100 mg, 0.247 mmol) in EtOH/AcOH(5 mL/1 mL) was added cyclopropanecarbaldehyde (86 mg, 1.235 mmol). The reaction mixture was stirred at 70° C. for 16 h. The solvent was removed under reduced pressure and the obtained residue was purified by Prep-HPLC to afford the corresponding example 51 (40 mg, 34.53%). LCMS: [M+H]⁺=469.2. ¹H NMR (400 MHZ, DMSO-d₆) δ 8.80 (s, 1H), 7.62 (s, 2H), 7.36 (t, J=7.4 Hz, 1H), 7.31-7.18 (m, 4H), 7.03 (d, J=7.0 Hz, 2H), 5.11 (s, 2H), 2.78 (d, J=6.8 Hz, 2H), 2.16 (s, 3H), 1.25-1.08 (m, 1H), 0.57-0.36 (m, 2H), 0.21-0.16 (m, 2H).

### Example 52: Synthesis of 4-((7-cyclopropyl-2,6-dimethyl-1H-imidazo[4,5-c]pyridin-1-yl)methyl)-3,5-difluorobenzenesulfonamide

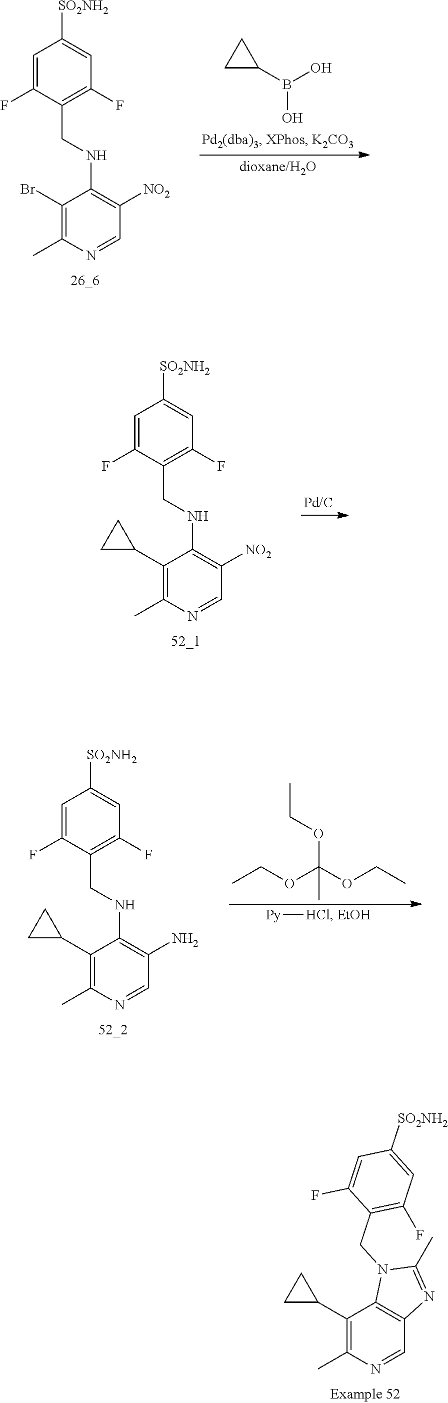

### Step 1: General procedure for preparation of 4-(((3-cyclopropyl-2-methyl-5-nitropyridin-4-yl)amino) methyl)-3,5-difluorobenzenesulfonamide (Compound 52_1)

To a solution of Compound 26_6 (500 mg, 1.14 mmol) in dioxane/H₂O (10 mL/1 mL) were added cyclopropylboronic acid (295 mg, 3.43 mmol), XPhos (57 mg, 0.12 mmol), Pd₂ (dba) 3 (55 mg, 0.06 mmol) and K₂CO₃ (474 mg, 3.43 mmol). This reaction mixture was stirred at 100° C. for 16 h under N₂ atmosphere. The solvent was removed under reduced pressure and the obtained residue was purified by flash silica gel chromatography to provide the corresponding Compound 52_1 (100 mg, 22.0% yield) as a white solid. LCMS: [M+H]⁺=399.1.

### Step 2: General procedure for preparation of 4-(((5-amino-3-cyclopropyl-2-methylpyridin-4-yl)amino) methyl)-3,5-difluorobenzenesulfonamide (Compound 52_2)

To a solution of Compound 52_1 (100 mg, 0.25 mmol) in methanol (5 mL) were added Pd/C (20 mg, 10% wt). The reaction was stirred at room temperature for 16 h under H₂ atmosphere. The mixture was filtered and the filter cake was washed with MeOH(15 mL×3). The filtrate was concentrated under reduced pressure. The obtained residue was purified by flash silica gel chromatography to provide the corresponding Compound 52_2 (75 mg, 81.1% yield) as a light yellow solid. LCMS: [M+H]⁺=369.1.

### Step 3: General procedure for preparation of 4-((7-cyclopropyl-2,6-dimethyl-1H-imidazo[4,5-c]pyridin-1-yl)methyl)-3,5-difluorobenzenesulfonamide

To a solution of Compound 52_2 (75 mg, 0.20 mmol) in ethanol (3 mL) was added 1,1,1-triethoxyethane (330 mg, 2.04 mmol) and Py-HCl (12 mg, 0.10 mmol). The reaction mixture was stirred at 80° C. for 2 h. The solvent was removed under reduced pressure and the obtained residue was purified by Prep-HPLC to afford the corresponding example 52 (20 mg, 25.0% yield). LCMS: [M+H]⁺=393.1. ¹H NMR: (400 MHZ, DMSO-d₆) δ 8.53 (s, 1H), 7.62 (s, 2H), 7.50 (d, J=7.5 Hz, 2H), 6.08 (s, 2H), 2.60 (s, 3H), 2.42 (s, 3H), 1.95-1.86 (m, 1H), 1.18-1.12 (m, 2H), 0.71-0.64 (m, 2H).

### Example 53: Synthesis of 6-((2-cyclopropyl-6-methyl-7-phenyl-1H-imidazo[4,5-c]pyridin-1-yl)methyl)pyridine-3-sulfonamide

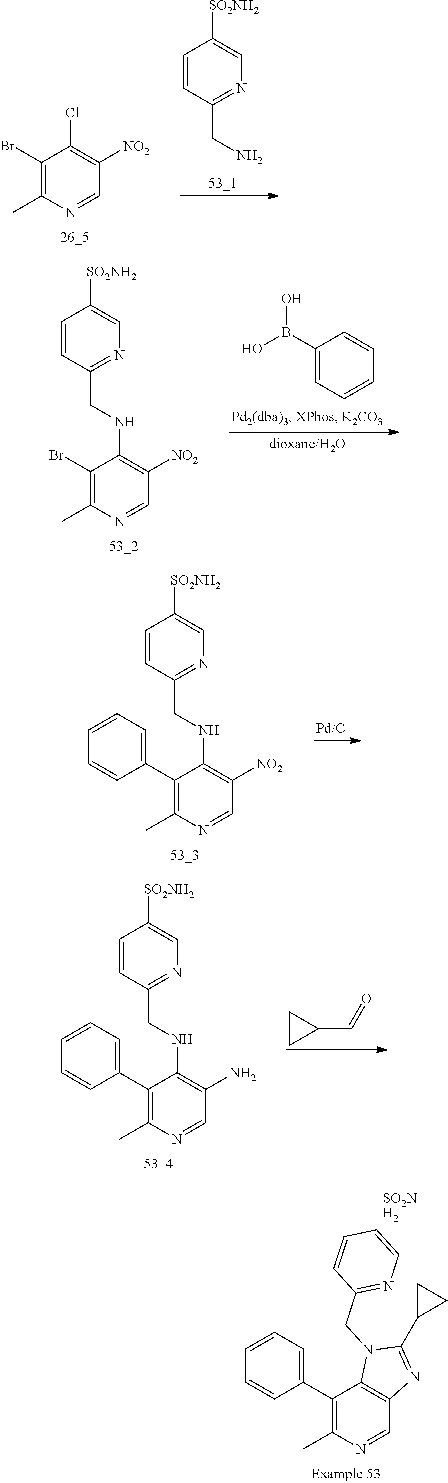

### Step 1: General procedure for preparation of 6-(((3-bromo-2-methyl-5-nitropyridin-4-yl)amino) methyl)pyridine-3-sulfonamide (Compound 53_2)

To a solution of Compound 26_5 (500 mg, 1.99 mmol) in acetonitrile (12 mL) were added Compound 53_1 (447 mg, 2.39 mmol) and DIPEA (770 mg, 5.97 mmol). This reaction mixture was stirred at 60° C. for 2 h. The solvent was removed under reduced pressure and the obtained residue was purified by flash silica gel chromatography to provide the corresponding Compound 53_2 (530 mg, 66.3% yield) as a yellow solid. LCMS: [M+H]⁺=402.0.

### Step 2: General procedure for preparation of 6-(((2-methyl-5-nitro-3-phenylpyridin-4-yl)amino) methyl)pyridine-3-sulfonamide (Compound 53_3)

To a solution of Compound 53_2 (530 mg, 1.32 mmol) in dioxane/H₂O (10 mL/1 mL) were added phenylboronic acid (241 mg, 1.98 mmol), XPhos (67 mg, 0.14 mmol), Pd₂ (dba) 3 (65 mg, 0.07 mmol) and K₂CO₃ (546 mg, 3.96 mmol). This reaction mixture was stirred at 100° C. for 16 h under N₂ atmosphere. The solvent was removed under reduced pressure and the obtained residue was purified by flash silica gel chromatography to provide the corresponding Compound 53_3 (430 mg, 81.7% yield) as a yellow solid. LCMS: [M+H]⁺=400.1.

### Step 3: General procedure for preparation of 6-(((5-amino-2-methyl-3-phenylpyridin-4-yl)amino) methyl)pyridine-3-sulfonamide (Compound 53_4)

To a solution of Compound 53_3 (100 mg, 0.25 mmol) in methanol (5 mL) were added Pd/C (20 mg, 10% wt). The reaction solution was stirred at room temperature for 16 h under H₂ atmosphere. The solution was filtered and the filter cake was washed with MeOH(15 mL×3). The filtrate was concentrated under reduced pressure. The obtained residue was purified by flash silica gel chromatography to provide the corresponding Compound 53_4 (60 mg, 64.9% yield) as a light yellow solid. LCMS: [M+H]⁺=370.1.

### Step 4: General procedure for preparation of 6-((2-cyclopropyl-6-methyl-7-phenyl-1H-imidazo[4,5-c]pyridin-1-yl)methyl)pyridine-3-sulfonamide

To a solution of Compound 53_4 (60 mg, 0.16 mmol) in ethanol (3 mL) were added cyclopropanecarbaldehyde (56 mg, 0.80 mmol) and AcOH(0.05 mL). The reaction mixture was stirred at 70° C. for 12 h. Solvent was removed under reduced pressure and the obtained residue was purified by Prep-HPLC to afford the corresponding example 53 (21 mg, 32.9% yield). LCMS: [M+H]⁺=420.2. ¹H NMR: (400 MHZ, DMSO-d₆) δ 8.73 (s, 1H), 8.68 (d, J=2.0 Hz, 1H), 8.02-7.94 (m, 1H), 7.59 (s, 2H), 7.34 (t, J=7.4 Hz, 1H), 7.23 (t, J=7.6 Hz, 2H), 6.98 (d, J=7.2 Hz, 2H), 6.70 (d, J=8.3 Hz, 1H), 5.15 (s, 2H), 2.16 (s, 3H), 2.07-1.98 (m, 1H), 1.05-0.97 (m, 2H), 0.96-0.89 (m, 2H).

### Example 54: Synthesis of 5-((2-cyclopropyl-6-methyl-7-phenyl-1H-imidazo[4,5-c]pyridin-1-yl)methyl)pyridine-2-sulfonamide

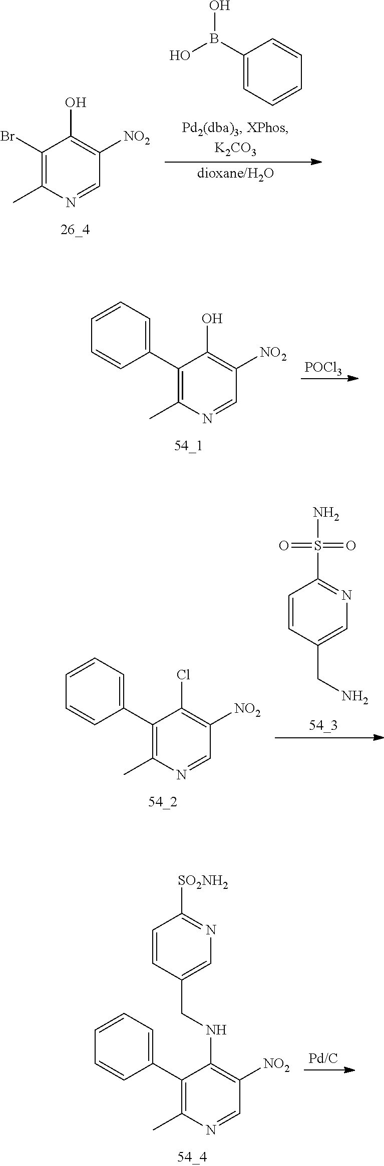

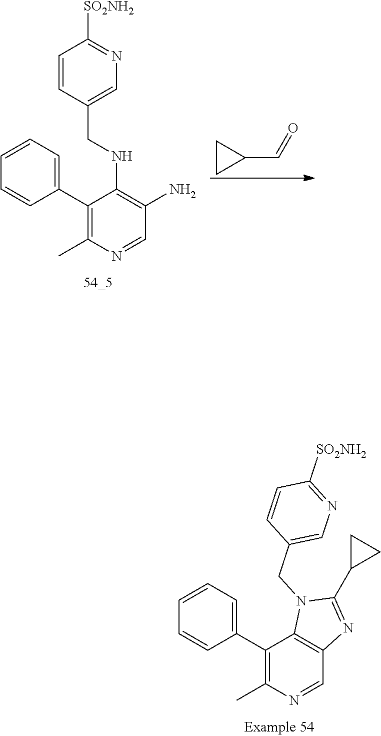

### Step 1: General procedure for preparation of 2-methyl-5-nitro-3-phenylpyridin-4-ol (Compound 54_1)

To a solution of Compound 26_4 (500 mg, 2.15 mmol) in dioxane/H₂O (10 mL/1 mL) were added phenylboronic acid (523 mg, 4.29 mmol), XPhos (105 mg, 0.22 mmol), Pd₂ (dba) 3 (101 mg, 0.11 mmol) and K₂CO₃ (890 mg, 6.45 mmol). This reaction mixture was stirred at 100° C. for 16 h under N₂ atmosphere. The solvent was removed under reduced pressure and the obtained residue was purified by flash silica gel chromatography to provide the corresponding Compound 54_1 (154 mg, 31.2% yield) as a white solid. LCMS: [M+H]⁺=231.1.

### Step 2: General procedure for preparation of 4-chloro-2-methyl-5-nitro-3-phenylpyridine (Compound 54_2)

To a solution of Compound 54_1 (154 mg, 0.67 mmol) in POCl₃ (5 mL) was added N,N-dimethylaniline (81 mg, 0.67 mmol) at 0° C. This reaction mixture was stirred at 100° C. for 1 h. The solvent was removed in vacuum to give the crude product which was purified by flash silica gel chromatography to afford Compound 54_2 (60 mg, 36.1% yield) as a white solid. LCMS: [M+H]⁺=249.1.

### Step 3: General procedure for preparation of 5-(((2-methyl-5-nitro-3-phenylpyridin-4-yl)amino) methyl)pyridine-2-sulfonamide (Compound 54_4)

To a solution of Compound 54_2 (60 mg, 0.24 mmol) in acetonitrile (5 mL) were added Compound 54_3 (54 mg, 0.29 mmol) and DIPEA (155 mg, 1.20 mmol). This reaction mixture was stirred at 60° C. for 2 h. The solvent was removed under reduced pressure and the obtained residue was purified by flash silica gel chromatography to provide the corresponding Compound 54_4 (40 mg, 41.5% yield) as a yellow solid. LCMS: [M+H]⁺=400.1.

### Step 4: General procedure for preparation of 5-(((5-amino-2-methyl-3-phenylpyridin-4-yl)amino) methyl)pyridine-2-sulfonamide (Compound 54_5)

To a solution of Compound 54_4 (40 mg, 0.10 mmol) in methanol (5 mL) were added Pd/C (20 mg, 10% wt). The reaction mixture was stirred at room temperature for 16 h under H₂ atmosphere. The reaction was filtered and the filter cake was washed with MeOH(15 mL×3). The filtrate was concentrated under reduced pressure. The obtained residue was purified by flash silica gel chromatography to provide the corresponding Compound 54_5 (20 mg, 54.1% yield) as a light yellow solid. LCMS: [M+H]⁺=370.1.

### Step 4: General procedure for preparation of 5-((2-cyclopropyl-6-methyl-7-phenyl-1H-imidazo[4,5-c]pyridin-1-yl)methyl)pyridine-2-sulfonamide

To a solution of Compound 54_5 (20 mg, 0.054 mmol) in ethanol (3 mL) were added cyclopropanecarbaldehyde (19 mg, 0.27 mmol) and AcOH(0.5 mL). The reaction mixture was stirred at 70° C. for 16 h. The solvent was removed under reduced pressure and the obtained residue was purified by Prep-HPLC to afford the corresponding example 54 (6 mg, 26.4% yield). LCMS: [M+H]⁺=420.2. ¹H NMR: (400 MHZ, DMSO-d₆) δ 8.75 (s, 1H), 7.87 (s, 1H), 7.72 (d, J=8.1 Hz, 1H), 7.43 (s, 2H), 7.37 (t, J=7.1 Hz, 1H), 7.27 (t, J=7.3 Hz, 2H), 7.11-7.05 (m, 3H), 5.14 (s, 2H), 2.18 (s, 3H), 2.11-1.04 (m, 1H), 1.10-1.03 (m, 2H), 0.98-0.92 (m, 2H).

### Example 55: Synthesis of 4-((2-cyclopropyl-6-methyl-7-phenyl-1H-imidazo[4,5-c]pyridin-1-yl)methyl)-2-methylbenzenesulfonamide

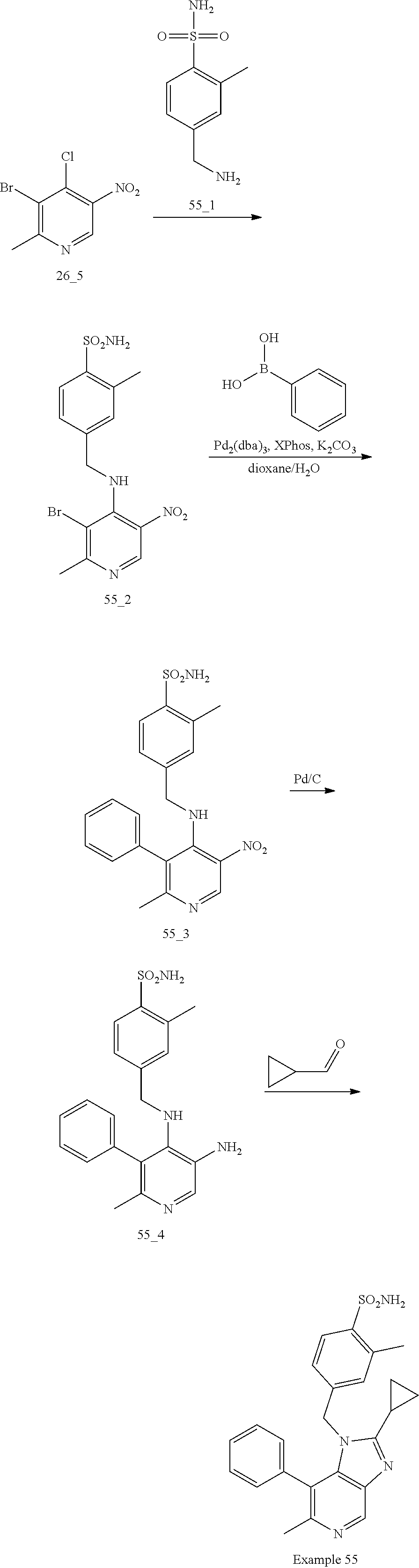

### Step 1: General procedure for preparation of 4-(((3-bromo-2-methyl-5-nitropyridin-4-yl)amino) methyl)-2-methylbenzenesulfonamide (Compound 55_2)

To a solution of Compound 26_5 (400 mg, 1.59 mmol) in acetonitrile (10 mL) were added Compound 55_1 (382 mg, 1.92 mmol) and DIPEA (615 mg, 4.77 mmol). This reaction mixture was stirred at 60° C. for 2 h. The solvent was removed under reduced pressure and the obtained residue was purified by flash silica gel chromatography to provide the corresponding Compound 55_2 (480 mg, 72.7% yield) as a yellow solid. LCMS: [M+H]⁺=415.0.

### Step 2: General procedure for preparation of 2-methyl-4-(((2-methyl-5-nitro-3-phenylpyridin-4-yl)amino) methyl)benzenesulfonamide (Compound 55_3)

To a solution of Compound 55_2 (480 mg, 1.16 mmol) in dioxane/H₂O (10 mL/1 mL) was added phenylboronic acid (211 mg, 1.73 mmol), XPhos (55 mg, 0.12 mmol), Pd₂ (dba) 3 (55 mg, 0.06 mmol) and K₂CO₃ (480 mg, 3.48 mmol). This reaction mixture was stirred at 100° C. for 16 h under N₂ atmosphere. The solvent was removed under reduced pressure and the obtained residue was purified by flash silica gel chromatography to provide the corresponding Compound 55_3 (380 mg, 79.7% yield) as a yellow solid. LCMS: [M+H]⁺=413.1.

### Step 3: General procedure for preparation of 4-(((5-amino-2-methyl-3-phenylpyridin-4-yl)amino) methyl)-2-methylbenzenesulfonamide (Compound 55_4)

To a solution of Compound 55_3 (100 mg, 0.24 mmol) in methanol (5 mL) were added Pd/C (20 mg, 10% wt). The reaction solution was stirred at room temperature for 16 h under H₂ atmosphere. The reaction solution was filtered and the filter cake was washed with MeOH(15 mL×3). The filtrate was concentrated under reduced pressure. The obtained residue was purified by flash silica gel chromatography to provide the corresponding Compound 55_4 (80 mg, 86.3% yield) as a light yellow solid. LCMS: [M+H]⁺=383.1.

### Step 4: General procedure for preparation of 4-((2-cyclopropyl-6-methyl-7-phenyl-1H-imidazo[4,5-c]pyridin-1-yl)methyl)-2-methylbenzenesulfonamide

To a solution of Compound 55_4 (80 mg, 0.21 mmol) in ethanol (5 mL) were cyclopropanecarbaldehyde (73 mg, 1.05 mmol) and AcOH(1.0 mL). The reaction mixture was stirred at 70° C. for 16 h. The solvent was removed under reduced pressure and the obtained residue was purified by Prep-HPLC to afford the corresponding example 55 (32 mg, 35.4% yield). LCMS: [M+H]⁺=433.2. ¹H NMR: (400 MHZ, DMSO-d₆) δ 8.73 (s, 1H), 7.63 (d, J=8.1 Hz, 1H), 7.43-7.22 (m, 5H), 7.07 (d, J=7.1 Hz, 2H), 6.47 (s, 1H), 6.42 (d, J=8.2 Hz, 1H), 5.02 (s, 2H), 2.44 (s, 3H), 2.17 (s, 3H), 2.04-1.96 (m, 1H), 1.08-1.00 (m, 2H), 0.98-0.92 (m, 2H).

### Example 56: Synthesis of 3,5-difluoro-4-((6-methoxy-3-methyl-2-oxo-7-phenyl-2,3-dihydro-1H-imidazo[4,5-c]pyridin-1-yl)methyl)benzenesulfonamide

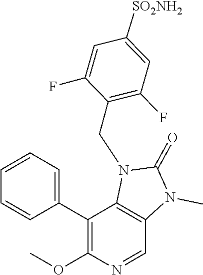 [SMILES: COc1ncc2c(c1-c1ccccc1)n(Cc1c(F)cc(S(N)(=O)=O)cc1F)c(=O)n2C]

The title compound was synthesized as described in example 19. LCMS: [M+H]⁺=461.2. ¹H NMR: (400 MHZ, DMSO-d₆) δ 8.00 (s, 1H), 7.59 (s, 2H), 7.36-7.18 (m, 5H), 7.07 (d, J=6.9 Hz, 2H), 4.74 (s, 2H), 3.70 (s, 3H), 3.39 (s, 3H).

### Example 57: Synthesis of 3,5-difluoro-4-((6-methoxy-2-oxo-7-phenyl-2,3-dihydro-1H-pyrrolo[3,2-c]pyridin-1-yl)methyl)benzenesulfonamide

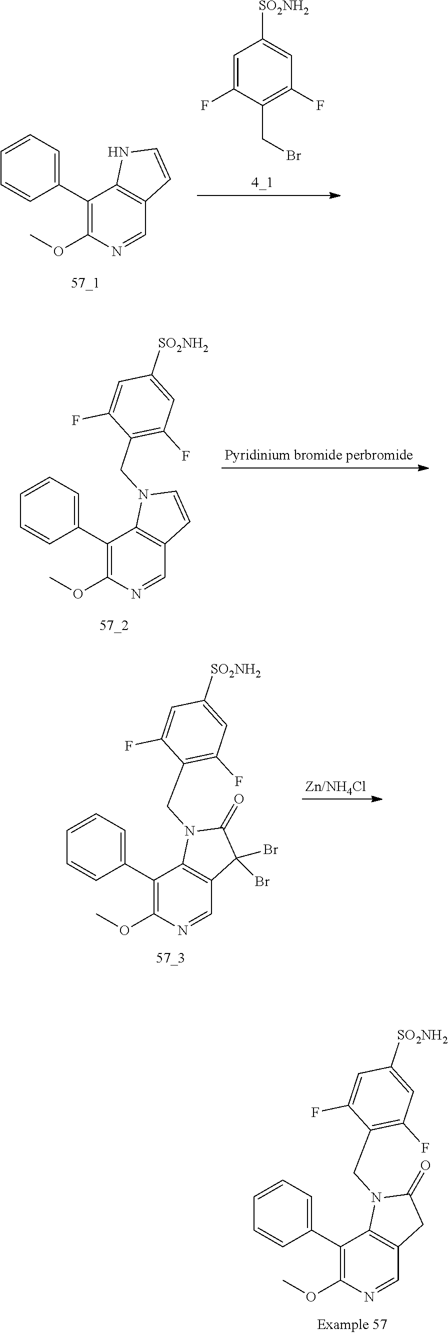 [SMILES: COc1ncc2cc[nH]c2c1-c1ccccc1 ; NS(=O)(=O)c1cc(F)c(CBr)c(F)c1 ; COc1ncc2ccn(Cc3c(F)cc(S(N)(=O)=O)cc3F)c2c1-c1ccccc1 ; COc1ncc2c(c1-c1ccccc1)N(Cc1c(F)cc(S(N)(=O)=O)cc1F)C(=O)C2(Br)Br ; COc1ncc2c(c1-c1ccccc1)N(Cc1c(F)cc(S(N)(=O)=O)cc1F)C(=O)C2]

### Step 1: General procedure for preparation of 3,5-difluoro-4-((6-methoxy-7-phenyl-1H-pyrrolo[3,2-c]pyridin-1-yl)methyl)benzenesulfonamide (Compound 57_2)

To a solution of Compound 57_1 (350 mg, 1.56 mmol) in DMF (5 mL) was added NaH(44 mg, 1.11 mmol, 60% in oil) at 0° C. The mixture was stirred at 0° C. for 0.5 h under N₂ atmosphere. Then Compound 4_1 (536 mg, 1.87 mmol) was added. The reaction mixture was stirred at room temperature for 2 h. After the reaction mixture was diluted with water (20 mL), the aqueous phase was extracted with ethyl acetate (20 mL×2). The combined organic phase was washed by brine, dried over anhydrous sodium sulfate, filtered, and concentrated in vacuum. The obtained residue purified by flash silica gel chromatography to provide the corresponding Compound 57_2 (260 mg, 38.8% yield) as a light yellow solid. LCMS: [M+H]⁺=430.1.

### Step 2: General procedure for preparation of 4-((3,3-dibromo-6-methoxy-2-oxo-7-phenyl-2,3-dihydro-1H-pyrrolo[3,2-c]pyridin-1-yl)methyl)-3,5-difluorobenzenesulfonamide (Compound 57_3)

To a solution of Compound 57_2 (260 mg, 0.61 mmol) in i-BuOH(10 mL) was added pyridinium tribromide (586 mg, 1.83 mmol) portion wise at 0° C. The reaction mixture was stirred at room temperature for 6 h, then quenched with H₂O (100 mL). The mixture was stirred for 10 minutes and extracted with ethyl acetate (20 mL×2). The combined organic layer was separated and washed with brine (20 mL×2), dried over anhydrous sodium sulfate, filtered, and concentrated in vacuum. The obtained residue purified by flash silica gel chromatography to provide the corresponding Compound 57_3 (120 mg, 32.9% yield) as a light yellow solid. LCMS: [M+H]⁺=602.1.

### Step 3: General procedure for preparation of 3,5-difluoro-4-((6-methoxy-2-oxo-7-phenyl-2,3-dihydro-1H-pyrrolo[3,2-c]pyridin-1-yl)methyl)benzenesulfonamide

To a solution of Compound 57_3 (120 mg, 0.20 mmol) in THF (10 mL) were added Zn power (130 mg, 2.0 mmol) and ammonium chloride (212 mg, 2.0 mmol). The mixture was stirred at 60° C. for 16 h. The mixture was filtered and the filtrate was concentrated in vacuum. The obtained residue was purified by Prep-HPLC to afford the corresponding example 57 (46 mg, 51.9% yield). LCMS: [M+H]⁺=446.2. ¹H NMR: (400 MHZ, DMSO-d₆) δ 7.98 (s, 1H), 7.53 (s, 2H), 7.36-7.16 (m, 5H), 7.03 (d, J=7.7 Hz, 2H), 4.54 (s, 2H), 3.69 (s, 2H), 3.69 (s, 3H).

### Example 58: Synthesis of 4-((7-(cyclopent-1-en-1-yl)-2,6-dimethyl-1H-imidazo[4,5-c]pyridin-1-yl)methyl)-3,5-difluorobenzenesulfonamide

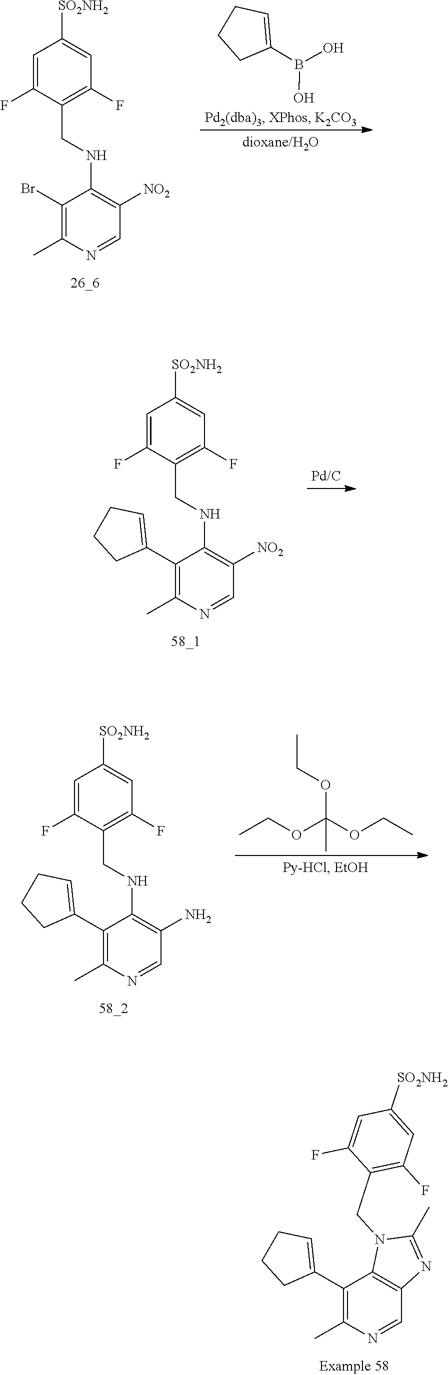 [SMILES: Cc1ncc([N+](=O)[O-])c(NCc2c(F)cc(S(N)(=O)=O)cc2F)c1Br ; OB(O)C1=CCCC1 ; Cc1ncc([N+](=O)[O-])c(NCc2c(F)cc(S(N)(=O)=O)cc2F)c1C1=CCCC1 ; Cc1ncc(N)c(NCc2c(F)cc(S(N)(=O)=O)cc2F)c1C1=CCCC1 ; CCOC(C)(OCC)OCC ; Cc1ncc2nc(C)n(Cc3c(F)cc(S(N)(=O)=O)cc3F)c2c1C1=CCCC1]

### Step 1: General procedure for preparation of 4-(((3-(cyclopent-1-en-1-yl)-2-methyl-5-nitropyridin-4-yl)amino) methyl)-3,5-difluorobenzenesulfonamide (Compound 58_1)

To a solution of Compound 26_6 (500 mg, 1.14 mmol) in dioxane/H₂O (10 mL/1 mL) were added cyclopent-1-en-1-ylboronic acid (384 mg, 3.43 mmol), XPhos (57 mg, 0.12 mmol), Pd₂ (dba) 3 (55 mg, 0.06 mmol) and K₂CO₃ (474 mg, 3.43 mmol). This reaction mixture was stirred at 100° C. for 16 h under N₂ atmosphere. The solvent was removed under reduced pressure and the obtained residue was purified by flash silica gel chromatography to provide the corresponding Compound 58_1 (130 mg, 26.8% yield) as a yellow solid. LCMS: [M+H]⁺=425.1.

### Step 2: General procedure for preparation of 4-(((5-amino-3-(cyclopent-1-en-1-yl)-2-methylpyridin-4-yl)amino) methyl)-3,5-difluorobenzenesulfonamide (Compound 58_2)

To a solution of Compound 58_1 (130 mg, 0.31 mmol) in methanol (5 mL) were added Pd/C (20 mg, 10% wt). The reaction was stirred at room temperature for 16 hr under H₂ atmosphere. The reaction solution was filtered and the filter cake was washed with MeOH(15 mL×3). The filtrate was concentrated under reduced pressure. The obtained residue was purified by flash silica gel chromatography to provide the corresponding Compound 58_2 (80 mg, 66.2% yield) as a light yellow solid. LCMS: [M+H]⁺=395.2.

### Step 3: General procedure for preparation of 4-((7-(cyclopent-1-en-1-yl)-2,6-dimethyl-1H-imidazo[4,5-c]pyridin-1-yl)methyl)-3,5-difluorobenzenesulfonamide

To a solution of Compound 58_2 (80 mg, 0.20 mmol) in ethanol (3 mL) were added 1,1,1-triethoxyethane (330 mg, 2.04 mmol) and Py-HCl (12 mg, 0.10 mmol). The reaction mixture was stirred at 80° C. for 2 h. The solvent was removed under reduced pressure and the residue was purified by Prep-HPLC to afford the corresponding example 58 (15 mg, 17.6% yield). LCMS: [M+H]⁺=419.2. ¹H NMR: (400 MHZ, DMSO-d₆) δ 8.62 (s, 1H), 7.64 (s, 2H), 7.48 (d, J=7.8 Hz, 2H), 5.75-5.41 (m, 3H), 2.60-2.59 (m, 2H), 2.48 (s, 3H), 2.37 (s, 3H), 2.18-1.80 (m, 4H).

### Example 59: Synthesis of 4-((3,6-dimethyl-2-oxo-7-phenyl-2,3-dihydro-1H-imidazo[4,5-c]pyridin-1-yl)methyl)-3,5-difluorobenzenesulfonamide

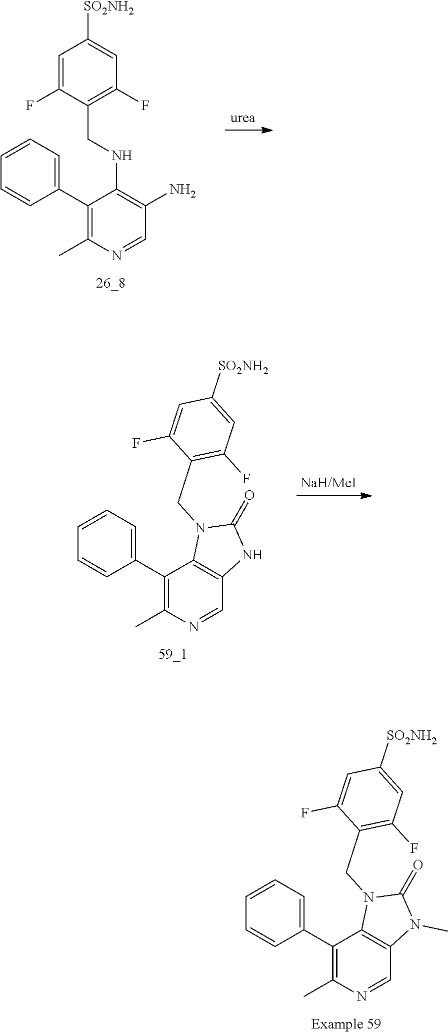 [SMILES: Cc1ncc(N)c(NCc2c(F)cc(S(N)(=O)=O)cc2F)c1-c1ccccc1 ; Cc1ncc2[nH]c(=O)n(Cc3c(F)cc(S(N)(=O)=O)cc3F)c2c1-c1ccccc1 ; Cc1ncc2c(c1-c1ccccc1)n(Cc1c(F)cc(S(N)(=O)=O)cc1F)c(=O)n2C]

### Step 1: General procedure for preparation of 3,5-difluoro-4-((6-methyl-2-oxo-7-phenyl-2,3-dihydro-1H-imidazo[4,5-c]pyridin-1-yl)methyl)benzenesulfonamide (Compound 59_1)

A mixture of Compound 26_8 (200 mg, 0.49 mmol) and urea (588 mg, 9.8 mmol) in a 3-necked round-bottomed flask was heated to 150° C. and stirred at this temperature for 3 h. Then the reaction mixture was diluted with water (10 mL) and stirred for 1 h at room temperature. The precipitate was filtered off and the filter cake was dried to give the crude product Compound 59_1 (70 mg, 32.9% yield) as a yellow solid. The crude product was used in the next step without further purification. LCMS: [M+H]⁺=431.1.

### Step 2: General procedure for preparation of 4-((3,6-dimethyl-2-oxo-7-phenyl-2,3-dihydro-1H-imidazo[4,5-c]pyridin-1-yl)methyl)-3,5-difluorobenzenesulfonamide

To a solution of Compound 59_1 (70 mg, 0.16 mmol) in DMF (5 mL) were added NaH(13 mg, 0.32 mmol, 60% in oil) under an ice bath. The mixture was stirred at this temperature for 30 mins. Methyl iodide (50 mg, 0.35 mmol) was added. The reaction mixture was stirred at room temperature for 2 h. The reaction mixture was quenched with water (10 mL). The aqueous phase was extracted with ethyl acetate (10 mL×2). The combined organic phase was washed by brine (10 mL), dried over anhydrous sodium sulfate, filtered, and concentrated in vacuum. The obtained residue was purified by Prep-HPLC to afford the corresponding example 59 (18 mg, 24.9% yield). LCMS: [M+H]⁺=445.2. ¹H NMR: (400 MHZ, DMSO-d₆) δ 8.34 (s, 1H), 7.61 (s, 2H), 7.40-7.23 (m, 5H), 7.09 (d, J=7.2 Hz, 2H), 4.67 (s, 2H), 3.42 (s, 3H), 2.10 (s, 3H).

### Example 60: Synthesis of 3,5-difluoro-4-((6-methyl-7-phenyl-2-(pyrrolidin-1-ylmethyl)-1H-imidazo[4,5-c]pyridin-1-yl)methyl)benzenesulfonamide

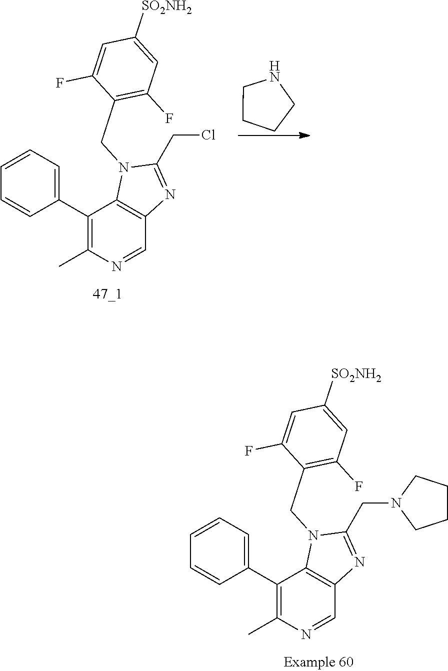

To a solution of Compound 47_1 (60 mg, 0.13 mmol) in THF (3 mL) was added pyrrolidine (46 mg, 0.65 mmol). The reaction was stirred at 60° C. for 1 h. The solvent was removed under reduced pressured and the obtained residue was purified by Prep-HPLC to afford the corresponding example 60 (30 mg, 46.5%). LCMS: [M+H]⁺=498.3.1H NMR (400 MHZ, DMSO-d₆) δ 8.81 (s, 1H), 7.59 (s, 2H), 7.37-7.20 (m, 5H), 7.11 (d, J=7.7 Hz, 2H), 5.19 (s, 2H), 3.83 (s, 2H), 2.40-2.34 (m, 4H), 2.19 (s, 3H), 1.51-1.45 (m, 4H).

### Example 61: Synthesis of 4-((7-(cyclopent-1-en-1-yl)-6-methoxy-3-methyl-2-oxo-2,3-dihydro-1H-imidazo[4,5-c]pyridin-1-yl)methyl)-3,5-difluorobenzenesulfonamide

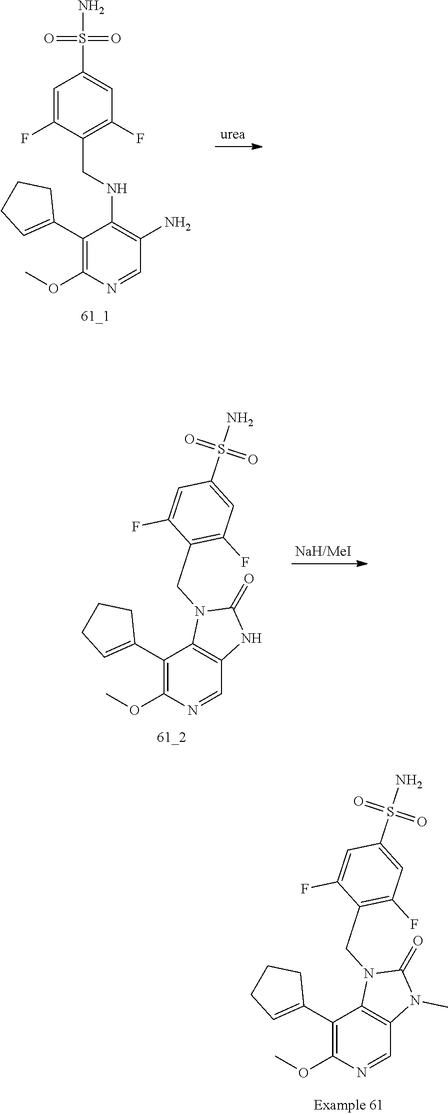 [SMILES: COc1ncc(N)c(NCc2c(F)cc(S(N)(=O)=O)cc2F)c1C1=CCCC1 ; COc1ncc2[nH]c(=O)n(Cc3c(F)cc(S(N)(=O)=O)cc3F)c2c1C1=CCCC1 ; COc1ncc2c(c1C1=CCCC1)n(Cc1c(F)cc(S(N)(=O)=O)cc1F)c(=O)n2C]

### Step 1: General procedure for preparation of 4-((7-(cyclopent-1-en-1-yl)-6-methoxy-2-oxo-2,3-dihydro-1H-imidazo[4,5-c]pyridin-1-yl)methyl)-3,5-difluorobenzenesulfonamide (Compound 61_2)

A mixture of Compound 61_1 (75.0 mg, 0.183 mmol) and urea (109.8 mg, 1.827 mmol) in a 3-necked round-bottomed flask was stirred at 160° C. for 1 h. Then the reaction mixture was diluted with water (10 mL) and stirred for 1 h at room temperature. The precipitate is filtered and the filter cake was dried to give the crude product Compound 61_2 (40.0 mg, 50.2% yield) as a yellow solid. The crude product was used in the next step without further purification. LCMS: [M+H]⁺=437.1.

### Step 2: General procedure for preparation of 4-((7-(cyclopent-1-en-1-yl)-6-methoxy-3-methyl-2-oxo-2,3-dihydro-1H-imidazo[4,5-c]pyridin-1-yl)methyl)-3,5-difluorobenzenesulfonamide

To a solution of Compound 61_2 (40.0 mg, 0.092 mmol) in DMF (5 mL) were added NaH(8 mg, 0.18 mmol, 60% in oil) under an ice bath. The reaction mixture was stirred at this temperature for 30 mins. Methyl iodide (39 mg, 0.28 mmol) was added. The reaction mixture was stirred at room temperature for 2 h. Then the reaction mixture was quenched with water (10 mL). The aqueous phase was extracted with ethyl acetate (10 mL×2). The combined organic phase was washed by brine (10 mL), dried over anhydrous sodium sulfate, filtered, and concentrated in vacuum. The obtained residue was purified by Prep-HPLC to afford the corresponding example 61 (2 mg, 4.8% yield) LCMS: [M+H]⁺=451.2. ¹H NMR: (400 MHz, DMSO-d₆) δ 7.89 (s, 1H), 7.63 (s, 2H), 7.45 (d, J=7.2 Hz, 2H), 5.64 (s, 1H), 5.22 (s, 2H), 3.78 (s, 3H), 2.51 (s, 3H), 2.50-2.34 (m, 4H), 1.86-1.72 (m, 2H).

### Example 62: Synthesis of 4-((7-(cyclopent-1-en-1-yl)-3,6-dimethyl-2-oxo-2,3-dihydro-1H-imidazo[4,5-c]pyridin-1-yl)methyl)-3,5-difluorobenzenesulfonamide

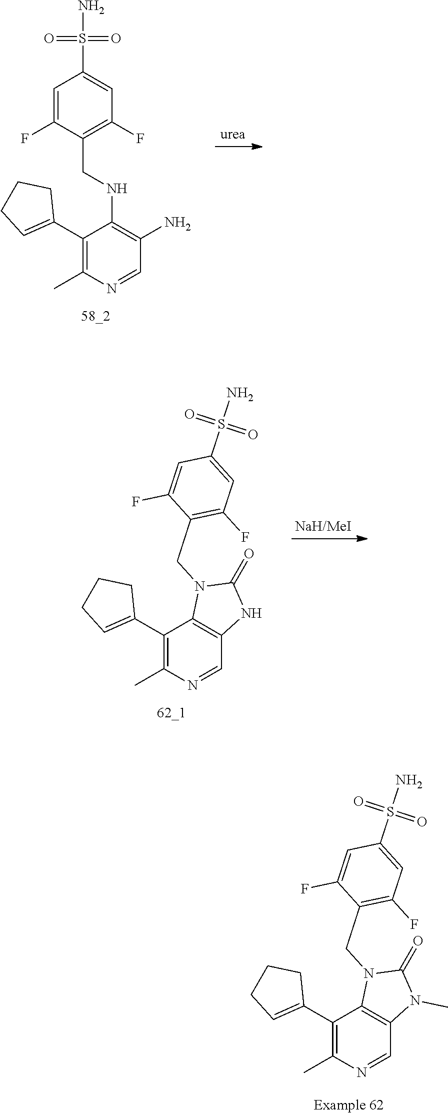 [SMILES: Cc1ncc(N)c(NCc2c(F)cc(S(N)(=O)=O)cc2F)c1C1=CCCC1 ; Cc1ncc2[nH]c(=O)n(Cc3c(F)cc(S(N)(=O)=O)cc3F)c2c1C1=CCCC1 ; Cc1ncc2c(c1C1=CCCC1)n(Cc1c(F)cc(S(N)(=O)=O)cc1F)c(=O)n2C]

### Step 1: General procedure for preparation of 4-((7-(cyclopent-1-en-1-yl)-6-methyl-2-oxo-2,3-dihydro-1H-imidazo[4,5-c]pyridin-1-yl)methyl)-3,5-difluorobenzenesulfonamide (Compound 62_1)

A mixture of Compound 58_2 (80 mg, 0.20 mmol) and urea (2.4 g, 40 mmol) in a 3-necked round-bottomed flask was stirred at 160° C. for 1 h. Then the reaction mixture was diluted with water (10 mL) and stirred for 1 h at room temperature. The precipitate is filtered and the filter cake was dried to give the crude product Compound 62_1 (30 mg, 35.2% yield) as a yellow solid. The crude product was used in the next step without further purification. LCMS: [M+H]⁺=421.1.

### Step 2: General procedure for preparation of 4-((7-(cyclopent-1-en-1-yl)-3,6-dimethyl-2-oxo-2,3-dihydro-1H-imidazo[4,5-c]pyridin-1-yl)methyl)-3,5-difluorobenzenesulfonamide

To a solution of Compound 62_1 (30 mg, 0.071 mmol) in DMF (5 mL) were added NaH(6 mg, 0.14 mmol, 60% in oil) under an ice bath. The reaction mixture was stirred at this temperature for 30 mins. Then methyl iodide (20 mg, 0.14 mmol) was added. The reaction mixture was stirred at room temperature for 2 h. Then the reaction mixture was quenched with water (10 mL). The aqueous phase was extracted with ethyl acetate (10 mL×2). The combined organic phase was washed by brine (10 mL), dried over anhydrous sodium sulfate, filtered, and concentrated in vacuum. The obtained residue was purified by Prep-HPLC to afford the corresponding example 62 (8 mg, 25.8% yield). LCMS: [M+H]⁺=435.2. ¹H NMR: (400 MHZ, DMSO-d₆) δ 8.22 (s, 1H), 7.61 (s, 2H), 7.45 (d, J=5.6 Hz, 2H), 5.55 (s, 1H), 5.27-5.07 (m, 2H), 3.36 (s, 3H), 2.70-2.64 (m, 2H), 2.38-2.26 (m, 1H), 2.32 (s, 3H), 2.21-1.71 (m, 3H).

### Example 63: Synthesis of 3,5-difluoro-4-((2,6,7-trimethyl-1H-imidazo[4,5-c]pyridin-1-yl)methyl)benzenesulfonamide

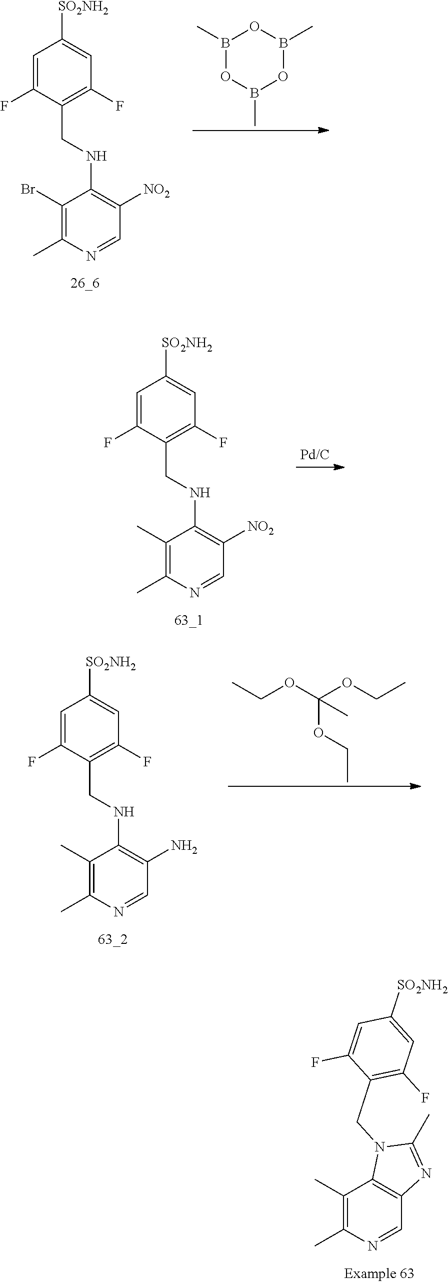

### Step 1: General procedure for preparation of 4-(((2,3-dimethyl-5-nitropyridin-4-yl)amino) methyl)-3,5-difluorobenzenesulfonamide (Compound 63_1)

To a solution of Compound 26_6 (500 mg, 1.14 mmol) in dioxane/H₂O (10 mL/1 mL) were added 2,4,6-trimethyl-1,3,5,2,4,6-trioxatriborinane (432 mg, 3.43 mmol), XPhos (57 mg, 0.12 mmol), Pd₂ (dba) 3 (55 mg, 0.06 mmol) and K₂CO₃ (474 mg, 3.43 mmol). This reaction mixture was stirred at 100° C. for 16 h under N₂ atmosphere. The solvent was removed under reduced pressure. The obtained residue was purified by flash silica gel chromatography to provide the corresponding Compound 63_1 (180 mg, 42.3% yield) as a yellow solid. LCMS: [M+H]⁺=373.1.

### Step 2: General procedure for preparation of 4-(((5-amino-2,3-dimethylpyridin-4-yl)amino) methyl)-3,5-difluorobenzenesulfonamide (Compound 63_2)

To a solution of Compound 63_1 (180 mg, 0.48 mmol) in methanol (5 mL) were added Pd/C (30 mg, 10% wt). The reaction was stirred at room temperature for 16 h under H₂ atmosphere. The reaction solution was filtered and the filter cake was washed with MeOH(15 mL×3). The filtrate was concentrated under reduced pressure. The obtained residue was purified by flash silica gel chromatography to provide the corresponding Compound 63_2 (90 mg, 54.4% yield) as a light yellow solid. LCMS: [M+H]⁺=343.1.

### Step 3: General procedure for preparation of 3,5-difluoro-4-((2,6,7-trimethyl-1H-imidazo[4,5-c]pyridin-1-yl)methyl)benzenesulfonamide

To a solution of Compound 63_2 (90 mg, 0.26 mmol) in ethanol (3 mL) were added 1,1,1-triethoxyethane (426 mg, 2.63 mmol) and Py-HCl (12 mg, 0.10 mmol). The reaction mixture was stirred at 80° C.° C. for 2 h. Solvent was removed under reduced pressure. The obtained residue was purified by Prep-HPLC to afford the corresponding example 63 (13 mg, 13.5% yield). LCMS: [M+H]⁺=367.2. ¹H NMR: (400 MHZ, DMSO-d₆) δ 8.50 (s, 1H), 7.62 (s, 2H), 7.52 (d, J=7.2 Hz, 2H), 5.80 (s, 2H), 2.51 (s, 3H), 2.47 (s, 3H), 2.39 (s, 3H).

### Example 64: Synthesis of 4-((2,6-dimethyl-7-(prop-1-yn-1-yl)-1H-imidazo[4,5-c]pyridin-1-yl)methyl)-3,5-difluorobenzenesulfonamide

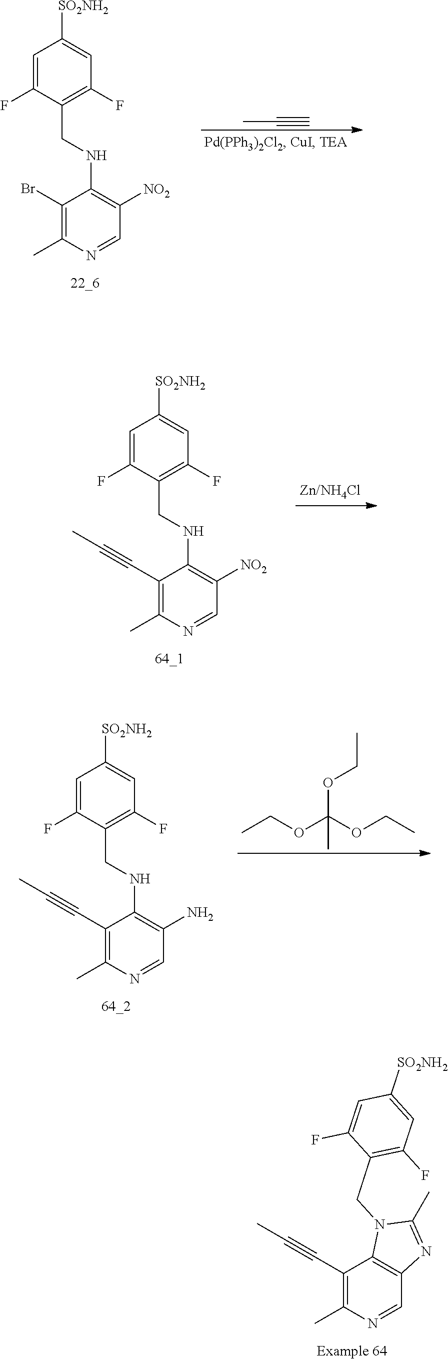

### Step 1: General procedure for preparation of 3,5-difluoro-4-(((2-methyl-5-nitro-3-(prop-1-yn-1-yl) pyridin-4-yl)amino) methyl)benzenesulfonamide (Compound 64_1)

To a solution of Compound 26_6 (500 mg, 1.14 mmol) in DMF (10 mL) were added prop-1-yne (5.7 mL, 1M in DMF, 5.70 mmol), CuI (108 mg, 0.57 mmol), Pd(PPh₃)₂Cl₂ (106 mg, 0.23 mmol), TBAI (85 mg, 0.23 mmol) and TEA (345 mg, 3.43 mmol). This reaction mixture was stirred at 110° C. for 16 h under N₂ atmosphere. Then the reaction mixture was diluted with water (20 mL). The aqueous phase was extracted with ethyl acetate (20 mL×2). The combined organic phase was washed by brine (20 mL), dried over anhydrous sodium sulfate, filtered, and concentrated in vacuum. The obtained residue was purified by flash silica gel chromatography to provide the corresponding Compound 64_1 (40 mg, 8.8% yield) as a yellow solid. LCMS: [M+H]⁺=397.1.

### Step 2: General procedure for preparation of 4-(((5-amino-2-methyl-3-(prop-1-yn-1-yl) pyridin-4-yl)amino) methyl)-3,5-difluorobenzenesulfonamide (Compound 64_2)

To a solution of Compound 64_1 (40 mg, 0.10 mmol) in ethanol (5 mL) were added Zn power (33 mg, 0.50 mmol) and NH₄Cl (53 mg, 1.0 mmol). The reaction mixture was stirred at 80° C. for 6 h. The reaction mixture was filtered and the filter cake was washed with MeOH(15 mL×3). The filtrate was concentrated under reduced pressure. The obtained residue was purified by flash silica gel chromatography to provide the corresponding Compound 64_2 (25 mg, 67.6% yield) as a light yellow solid. LCMS: [M+H]⁺=367.1.

### Step 3: General procedure for preparation of 4-((2,6-dimethyl-7-(prop-1-yn-1-yl)-1H-imidazo[4,5-c]pyridin-1-yl)methyl)-3,5-difluorobenzenesulfonamide

To a solution of Compound 64_2 (25 mg, 0.068 mmol) in ethanol (3 mL) were added 1,1,1-triethoxyethane (111 mg, 0.68 mmol) and Py-HCl (6 mg, 0.05 mmol). The reaction mixture was stirred at 80° C. for 2 h. The solvent was removed under reduced pressure. The obtained residue was purified by Prep-HPLC to afford the corresponding example 64 (10 mg, 37.5% yield). LCMS: [M+H]⁺=391.2. ¹H NMR: (400 MHZ, DMSO-d₆) δ 9.06 (s, 1H), 7.67 (s, 2H), 7.55 (d, J=7.8 Hz, 2H), 6.03 (s, 2H), 2.70 (s, 3H), 2.60 (s, 3H), 2.12 (s, 3H).

### Example 65: Synthesis of 6-((2,6-dimethyl-7-phenyl-1H-imidazo[4,5-c]pyridin-1-yl)methyl)pyridine-3-sulfonamide

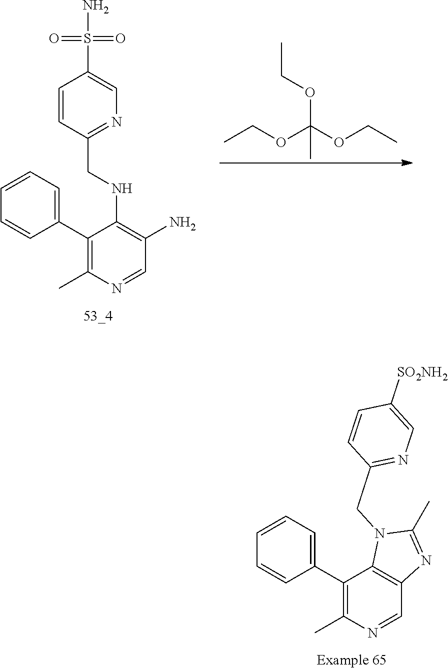 [SMILES: Cc1ncc(N)c(NCc2ccc(S(N)(=O)=O)cn2)c1-c1ccccc1 ; CCOC(C)(OCC)OCC ; Cc1ncc2nc(C)n(Cc3ccc(S(N)(=O)=O)cn3)c2c1-c1ccccc1]

To a solution of Compound 53_4 (33 mg, 0.089 mmol) in EtOH(1 mL) were added 1,1,1-triethoxyethane (145 mg, 0.89 mmol) and pyridine hydrochloride (4.45 mg, 0.039 mmol). The reaction mixture was stirred at 80° C. for 2 h. The solvent was removed under reduced pressure. The obtained residue was purified by Prep-HPLC to afford the corresponding example 65 (12 mg, 34.1%). LCMS: [M+H]⁺=394.2. ¹H NMR: (400 MHZ, DMSO-d₆) δ 8.77 (s, 1H), 8.66 (d, J=1.8 Hz, 1H), 7.97 (dd, J=8.2, 2.3 Hz, 1H), 7.58 (s, 2H), 7.35 (t, J=7.4 Hz, 1H), 7.24 (t, J=7.6 Hz, 2H), 6.96 (d, J=7.0 Hz, 2H), 6.65 (d, J=8.3 Hz, 1H), 5.01 (s, 2H), 2.43 (s, 3H), 2.16 (s, 3H).

### Example 66: Synthesis of 5-((2,6-dimethyl-7-phenyl-1H-imidazo[4,5-c]pyridin-1-yl)methyl)pyridine-2-sulfonamide

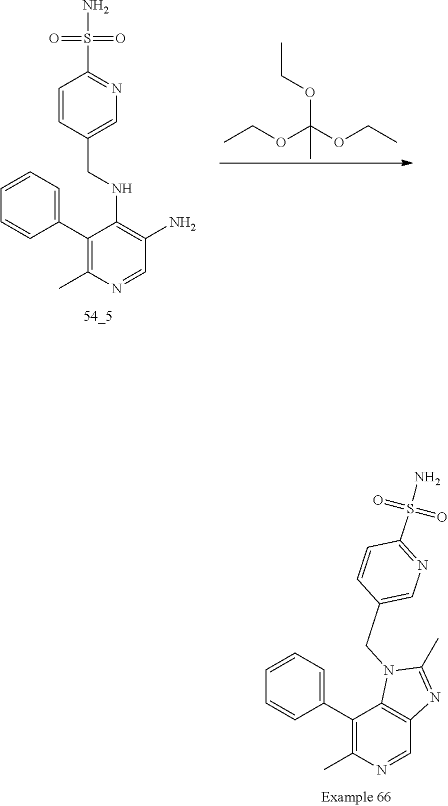 [SMILES: Cc1ncc(N)c(NCc2ccc(S(N)(=O)=O)nc2)c1-c1ccccc1 ; CCOC(C)(OCC)OCC ; Cc1ncc2nc(C)n(Cc3ccc(S(N)(=O)=O)nc3)c2c1-c1ccccc1]

To a solution of Compound 54_5 (40 mg, 0.11 mmol) in EtOH(3 mL) were added triethyl orthoacetate (52.7 mg, 0.325 mmol) and Py-HCl (1.3 mg, 0.011 mmol). The reaction mixture was stirred at 80° C. for 1 h. The reaction mixture was concentrated under reduced pressure. The residue was purified by Prep-HPLC to afford the corresponding example 66 (10.0 mg, 23.5% yield). LCMS: [M+H]⁺=394.2. ¹H NMR: (400 MHZ, DMSO-d₆) δ 8.79 (s, 1H), 7.80 (d, J=1.6 Hz, 1H), 7.69 (d, J=7.2 Hz, 1H), 7.39-7.33 (m, 1H), 7.30-7.23 (m, 2H), 7.18 (s, 2H), 7.08-C_{6.99} (m, 3H), 4.99 (s, 2H), 2.44 (s, 3H), 2.19 (s, 3H).

### Example 67: Synthesis of 6-((6-methyl-7-phenyl-2-(trifluoromethyl)-1H-imidazo[4,5-c]pyridin-1-yl)methyl)pyridine-3-sulfonamide

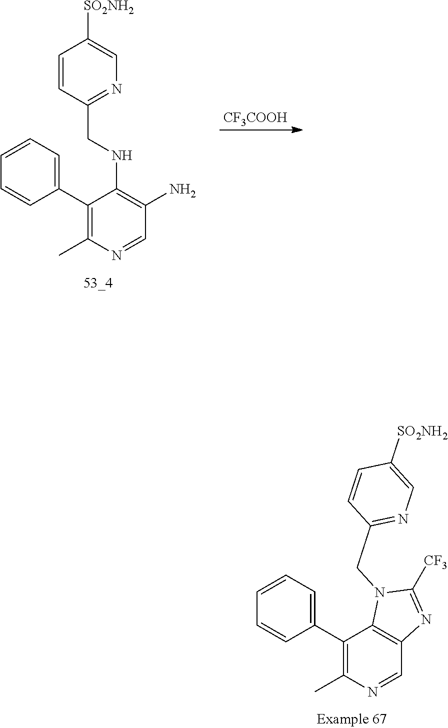 [SMILES: Cc1ncc(N)c(NCc2ccc(S(N)(=O)=O)cn2)c1-c1ccccc1 ; "CF3COOH" ; Cc1ncc2nc(C(F)(F)F)n(Cc3ccc(S(N)(=O)=O)cn3)c2c1-c1ccccc1]

A mixture of Compound 53_4 (100 mg, 0.27 mmol) in trifluoroacetic acid (5 mL) was stirred at 100° C.° C. for 3 h. The solvent was removed under reduced pressure. The residue was purified by was purified by Prep-HPLC to afford the corresponding example 67 (5.0 mg, 4.1%). LCMS: [M+H]⁺=448.1. ¹H NMR: (400 MHZ, DMSO-d₆) δ 9.13 (s, 1H), 8.61 (s, 1H), 7.96 (d, J=8.4 Hz, 1H), 7.58 (s, 2H), 7.34 (t, J=7.7 Hz, 1H), 7.22 (t, J=7.3 Hz, 2H), 6.98 (d, J=6.8 Hz, 2H), 6.85 (d, J=8.2 Hz, 1H), 5.22 (s, 2H), 2.22 (s, 3H).

### Example 68: Synthesis of 6-((2,6-dimethyl-7-phenyl-1H-imidazo[4,5-c]pyridin-1-yl)methyl)-5-fluoropyridine-3-sulfonamide

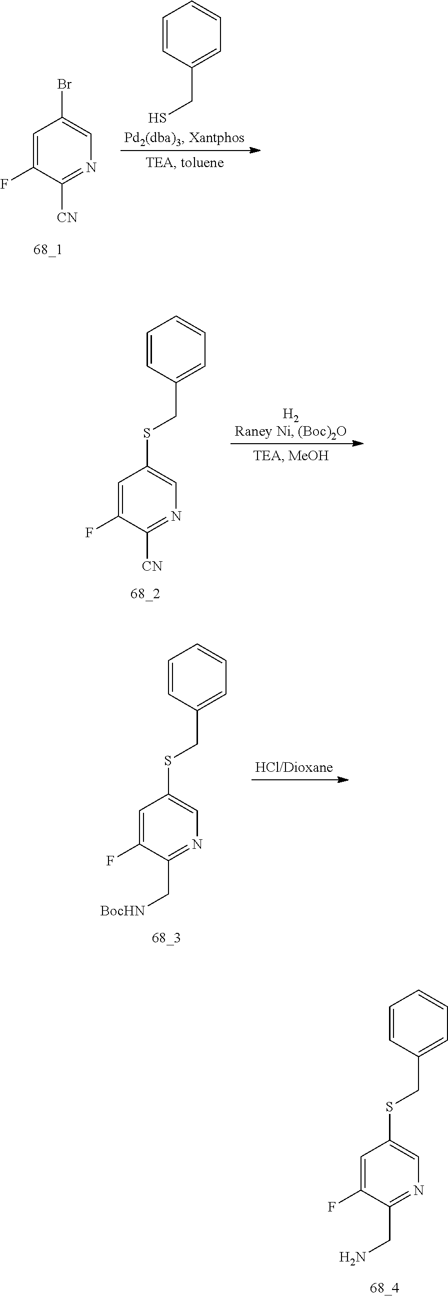 [SMILES: N#Cc1ncc(Br)cc1F ; SCc1ccccc1 ; N#Cc1ncc(SCc2ccccc2)cc1F ; CC(C)(C)OC(=O)NCc1ncc(SCc2ccccc2)cc1F ; NCc1ncc(SCc2ccccc2)cc1F]

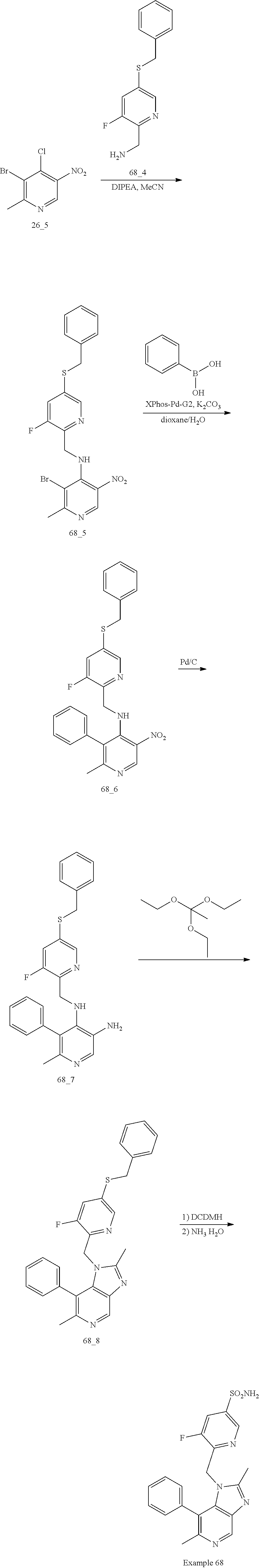

### Step 1: General Procedure for Preparation of 5-(Benzylthio)-3-Fluoropicolinonitrile (Compound 68_2)

To a solution of Compound 68_1 (10 g, 49.8 mmol) in toluene (100 mL) were added phenylmethanethiol (6.2 g, 49.8 mmol), Xantphos (1.73 g, 2.99 mmol), Pd₂ (dba) 3 (1.40 g, 1.50 mmol) and TEA (25.2 g, 249 mmol). This reaction mixture was stirred at 100° C. for 16 h under N₂ atmosphere. The solvent was removed under reduced pressure. The obtained residue was purified by flash silica gel chromatography to provide the corresponding Compound 68_2 (10.1 g, 83.1% yield) as a yellow solid. LCMS: [M+H]⁺=245.0.

### Step 2: General procedure for preparation of tert-butyl ((5-(benzylthio)-3-fluoropyridin-2-yl)methyl) carbamate (Compound 68_3)

To a solution of Compound 68_2 (10 g, 41.3 mmol) in methanol (80 mL) were added Raney-Ni (1 g, 16 mmol), (Boc)₂₀ (18 g, 82.6 mmol) and TEA (12.5 g, 123.9 mmol). The reaction was stirred at room temperature for 16 hr under H₂ (4 Mpa). The reaction mixture was filtered with Celite and the filter cake was washed with MeOH(50 mL×3). The filtrate was concentrated under reduced pressure. The obtained residue was purified by flash silica gel chromatography to provide the corresponding Compound 68_3 (6.2 g, 43.5% yield) as a white solid. LCMS: [M+H]⁺=349.1.

### Step 3: General Procedure for Preparation of ((5-(Benzylthio)-3-Fluoropyridin-2-Yl) Methanamine (Compound 68_4)

To a solution of Compound 68_3 (6.2 g, 17.8 mmol) in DCM (20 mL) was added HCl/dioxane (4 M, 10 mL). The reaction was stirred at room temperature for 3 h. The solvent was removed under reduced pressure to provide the corresponding Compound 68_4 (4.1 g, 92.8% yield) as a white solid. The crude product was used in the next step without further purification. LCMS: [M+H]⁺=249.1.

### Step 4: General procedure for preparation of N-((5-(benzylthio)-3-fluoropyridin-2-yl)methyl)-3-bromo-2-methyl-5-nitropyridin-4-amine (Compound 68_5)

To a solution of Compound 26_5 (200 mg, 0.75 mmol) in acetonitrile (10 mL) were added Compound 68_4 (186 mg, 0.75 mmol) and DIPEA (290 mg, 0.22 mmol). This reaction mixture was stirred at 60° C. for 4 h. The solvent was removed under reduced pressure. The obtained residue was purified by flash silica gel chromatography to provide the corresponding Compound 68_5 (260 mg, 72.5% yield) as a yellow solid. LCMS: [M+H]⁺=463.0.

### Step 5: General procedure for preparation of N-((5-(benzylthio)-3-fluoropyridin-2-yl)methyl)-2-methyl-5-nitro-3-phenylpyridin-4-amine (Compound 68_6)

To a solution of Compound 68_5 (200 mg, 0.43 mmol) in dioxane/H₂O (10 mL/1 mL) were added phenylboronic acid (79 mg, 0.65 mmol), XPhos-Pd-G2 (33 mg, 0.043 mmol) and K₂CO₃ (178 mg, 1.29 mmol). The reaction mixture was stirred under 100° C. for 16 h under N₂ atmosphere. The solvent concentrated under reduced pressure, The residue was purified by flash silica gel chromatography to provide the corresponding Compound 68_6 (160 mg, 80.5% yield) as a yellow solid. LCMS: [M+H]⁺=461.1.

### Step 6: General procedure for preparation of N₄-((5-(benzylthio)-3-fluoropyridin-2-yl)methyl)-6-methyl-5-phenylpyridine-3,4-diamine (Compound 68_7)

To a solution of Compound 68_6 (160 mg, 0.35 mmol) in MeOH(5 mL) were added DCM (5 mL) and Pd/C (30 mg, 10% wt). The reaction was stirred at room temperature for 16 hr under H₂ atmosphere. The reaction was filtered and the filter cake was washed with MeOH(5 mL×3). The filtrate was concentrated under reduced pressure. The obtained residue was purified by flash silica gel chromatography to provide the corresponding Compound 68_7 (150 mg, 89.6% yield) as a light yellow solid. LCMS: [M+H]⁺=431.1.

### Step 7: General procedure for preparation of 1-((5-(benzylthio)-3-fluoropyridin-2-yl)methyl)-2,6-dimethyl-7-phenyl-1H-imidazo[4,5-c]pyridine (Compound 68_8)

To a solution of Compound 68_7 (150 mg, 0.35 mmol) in EtOH(5 mL) were added triethyl orthoacetate (565 mg, 3.48 mmol) and Py-HCl (4 mg, 0.035 mmol). The reaction mixture was stirred at 100° C. for 1 h. The solvent was concentrated under reduced pressure. The residue was purified by flash silica gel chromatography to provide the corresponding Compound 68_8 (140 mg, 88.4% yield) as a yellow solid. LCMS: [M+H]⁺=455.2.

### Step 8: General procedure for preparation of 6-((2,6-dimethyl-7-phenyl-1H-imidazo[4,5-c]pyridin-1-yl)methyl)-5-fluoropyridine-3-sulfonamide

To a solution of Compound 68_8 (140 mg, 0.31 mmol) in acetonitrile (10 mL)/AcOH(0.3 mL)/H₂O (0.2 mL) at 0° C. was added drop wise a solution of 1,3-dichloro-5,5-dimethylhydantoin/MeCN (153 mg/2 mL, 0.78 mmol). The reaction mixture was stirred under 0° C. for 30 min. The mixture was diluted with water (10 mL) and extracted with DCM (20 mL×2). The combined organic layers were dried over anhydrous sodium sulfate, filtered, and concentrated under reduced pressure. The residue was dissolved in acetonitrile (5 mL) and ammonium hydroxide (0.5 mL) was added at 0° C. The reaction was stirred at 0° C. for 30 min. The reaction mixture was concentrated under reduced pressure. The obtained residue was purified by Prep-HPLC to afford the corresponding example 68 (30 mg, 23.4% yield). LCMS: [M+H]⁺=412.1. ¹H NMR: (400 MHZ, DMSO-d₆) δ 8.74 (s, 1H), 8.50 (s, 1H), 7.81 (dd, J=9.1, 1.7 Hz, 1H), 7.71 (s, 2H), 7.35 (t, J=7.5 Hz, 1H), 7.22 (t, J=7.6 Hz, 2H), 6.86 (d, J=7.1 Hz, 2H), 5.13 (s, 2H), 2.46 (s, 3H), 2.14 (s, 3H).

### Example 69: Synthesis of 5-fluoro-6-((6-methyl-7-phenyl-2-(trifluoromethyl)-1H-imidazo[4,5-c]pyridin-1-yl)methyl)pyridine-3-sulfonamide

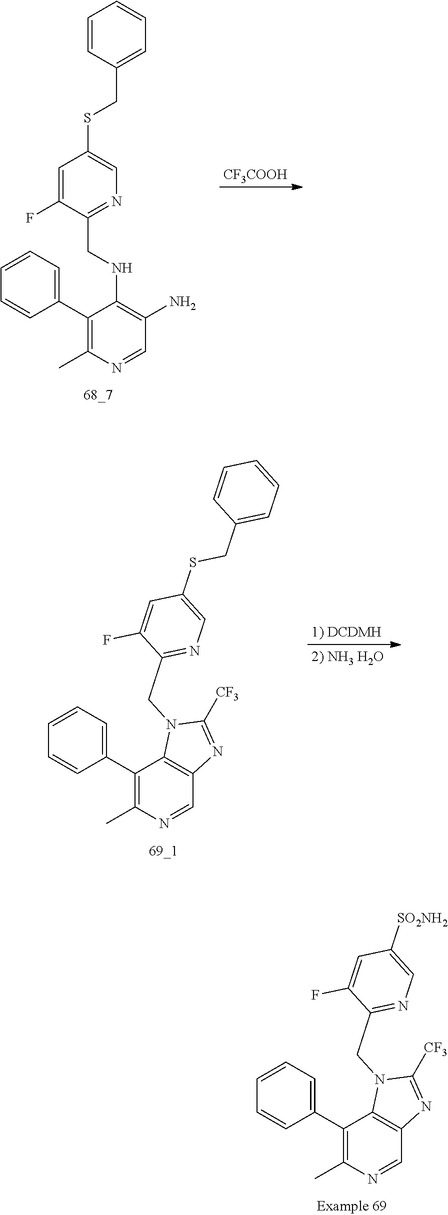 [SMILES: Cc1ncc(N)c(NCc2ncc(SCc3ccccc3)cc2F)c1-c1ccccc1 ; "CF3COOH" ; Cc1ncc2nc(C(F)(F)F)n(Cc3ncc(SCc4ccccc4)cc3F)c2c1-c1ccccc1 ; Cc1ncc2nc(C(F)(F)F)n(Cc3ncc(S(N)(=O)=O)cc3F)c2c1-c1ccccc1]

### Step 1: General procedure for preparation of 1-((5-(benzylthio)-3-fluoropyridin-2-yl)methyl)-6-methyl-7-phenyl-2-(trifluoromethyl)-1H-imidazo[4,5-c]pyridine (Compound 69_1)

To a solution of Compound 68_7 (150 mg, 0.35 mmol) in trifluoroacetic acid (5 mL). The reaction mixture was stirred at 100° C. for 3 h. The solvent was concentrated under reduced pressure. The obtained residue was purified by flash silica gel chromatography to provide the corresponding Compound 69_1 (120 mg, 67.7% yield) as a yellow solid. LCMS: [M+H]⁺=509.1.

### Step 2: General procedure for preparation of 5-fluoro-6-((6-methyl-7-phenyl-2-(trifluoromethyl)-1H-imidazo[4,5-c]pyridin-1-yl)methyl)pyridine-3-sulfonamide

To a solution of Compound 69_1 (120 mg, 0.24 mmol) in acetonitrile (10 mL)/AcOH(0.3 mL)/H₂O (0.2 mL) at 0° C. was dropwised added a solution of 1,3-dichloro-5,5-dimethylhydantoin/acetonitrile (118 mg/2 mL, 0.60 mmol). The reaction mixture was stirred under 0° C. for 30 min. The mixture was diluted with water (10 mL) and extracted with DCM (20 mL×2). The combined organic layers were dried over anhydrous sodium sulfate, filtered, and concentrated under reduced pressure. The residue was dissolved in acetonitrile (5 mL) and ammonium hydroxide (0.5 mL) was added at 0° C. The reaction mixture was concentrated under reduced pressure. The obtained residue was purified by Prep-HPLC to afford the corresponding example 69 (20 mg, 18.2% yield). LCMS: [M+H]⁺=466.2. ¹H NMR: (400 MHZ, DMSO-d₆) δ 9.12 (s, 1H), 8.49 (s, 1H), 7.88-7.80 (m, 1H), 7.70 (s, 2H), 7.38 (t, J=7.4 Hz, 1H), 7.24 (t, J=7.7 Hz, 2H), 6.93 (s, 2H), 5.34 (s, 2H), 2.20 (s, 3H).

### Example 70: Synthesis of 6-((2-cyclopropyl-6-methyl-7-phenyl-1H-imidazo[4,5-c]pyridin-1-yl)methyl)-5-fluoropyridine-3-sulfonamide

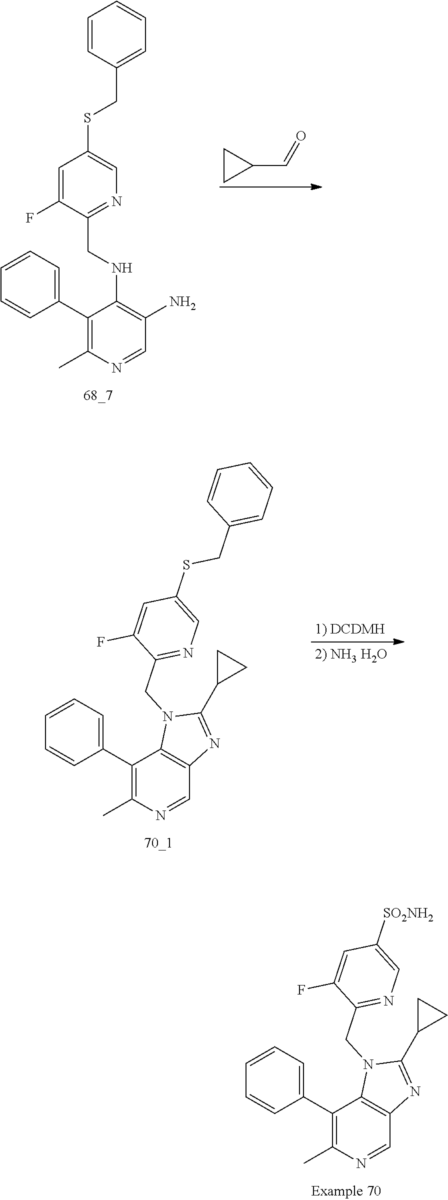 [SMILES: Cc1ncc(N)c(NCc2ncc(SCc3ccccc3)cc2F)c1-c1ccccc1 ; O=CC1CC1 ; Cc1ncc2nc(C3CC3)n(Cc3ncc(SCc4ccccc4)cc3F)c2c1-c1ccccc1 ; Cc1ncc2nc(C3CC3)n(Cc3ncc(S(N)(=O)=O)cc3F)c2c1-c1ccccc1]

### Step 1: General procedure for preparation of 1-((5-(benzylthio)-3-fluoropyridin-2-yl)methyl)-2-cyclopropyl-6-methyl-7-phenyl-1H-imidazo[4,5-c]pyridine (Compound 70_1)

To a solution of Compound 68_7 (150 mg, 0.35 mmol) in ethanol (3 mL) were added cyclopropanecarbaldehyde (123 mg, 1.75 mmol) and AcOH(0.1 mL). The reaction mixture was stirred at 70° C. for 12 h. The solvent was removed under reduced pressure. The obtained residue was purified by flash silica gel chromatography to afford the corresponding Compound 70_1 (80 mg, 47.8% yield) as a white solid. LCMS: [M+H]⁺=481.2.

### Step 2: General procedure for preparation of 6-((2-cyclopropyl-6-methyl-7-phenyl-1H-imidazo[4,5-c]pyridin-1-yl)methyl)-5-fluoropyridine-3-sulfonamide

To a solution of Compound 70_1 (80 mg, 0.17 mmol) in acetonitrile (10 mL)/AcOH(0.3 mL)/H₂O (0.2 mL) at 0° C. was dropwised added a solution of 1,3-dichloro-5,5-dimethylhydantoin/acetonitrile (84 mg/2 mL, 0.43 mmol). The reaction mixture was stirred under 0° C. for 30 min. The reaction mixture was quenched with water and the aqueous layer was extracted with DCM (20 mL×2). The organic layer was concentrated under reduced pressure. The residue was dissolved in acetonitrile (5 mL). The mixture was cooled to 0° C., and ammonium hydroxide (0.5 mL) was added. The reaction solution was stirred at 0° C. for 3 min. The solvent was removed under reduced pressure and the residue was purified by Prep-HPLC to afford the corresponding example 70 (12 mg, 16.5% yield). LCMS: [M+H]⁺=438.2. ¹H NMR: (400 MHZ, DMSO-d₆) δ 8.70 (s, 1H), 8.51 (s, 1H), 7.86-7.77 (m, 1H), 7.60 (s, 2H), 7.34 (t, J=7.5 Hz, 1H), 7.22 (t, J=7.6 Hz, 2H), 6.89 (d, J=7.2 Hz, 2H), 5.29 (s, 2H), 2.15-2.07 (m, 1H), 2.13 (s, 3H), 1.07-0.90 (m, 4H).

### Example 71: Synthesis of 6-((2-cyclopropyl-6-methyl-7-phenyl-1H-imidazo[4,5-c]pyridin-1-yl)methyl)pyridine-3-sulfonamide

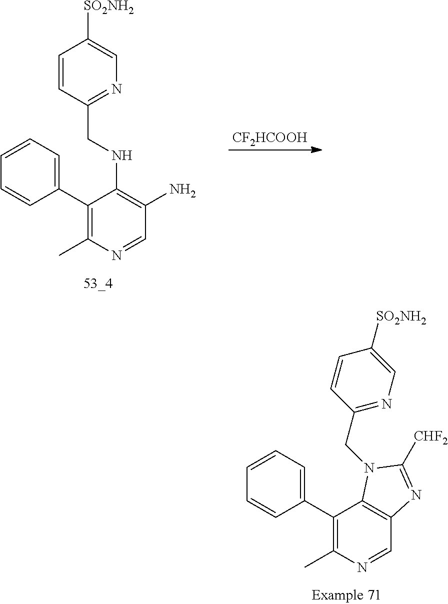 [SMILES: Cc1ncc(N)c(NCc2ccc(S(N)(=O)=O)cn2)c1-c1ccccc1 ; "CF2HCOOH" ; Cc1ncc2nc(C(F)F)n(Cc3ccc(S(N)(=O)=O)cn3)c2c1-c1ccccc1]

A solution of Compound 53_4 (80 mg, 0.22 mmol) in difluoroacetic acid (5 mL) was stirred at 100° C. for 3 h. The solvent was removed under reduced pressure. The obtained residue was purified by Prep-HPLC to provide the corresponding Compound 71 (10 mg, 11.0% yield). LCMS: [M+H]⁺=430.1. ¹H NMR: (400 MHZ, DMSO-d₆) δ 9.04 (s, 1H), 8.61 (d, J=1.8 Hz, 1H), 7.95 (dd, J=8.3, 2.3 Hz, 1H), 7.58 (s, 2H), 7.41 (t, J=51.6 Hz, 1H), 7.33 (t, J=7.6 Hz, 1H), 7.21 (t, J=7.7 Hz, 2H), 6.96 (d, J=7.0 Hz, 2H), 6.72 (d, J=8.1 Hz, 1H), 5.21 (s, 2H), 2.21 (s, 3H).

### Example 72: Synthesis of 6-((2-(difluoromethyl)-6-methyl-7-phenyl-1H-imidazo[4,5-c]pyridin-1-yl)methyl)-5-fluoropyridine-3-sulfonamide

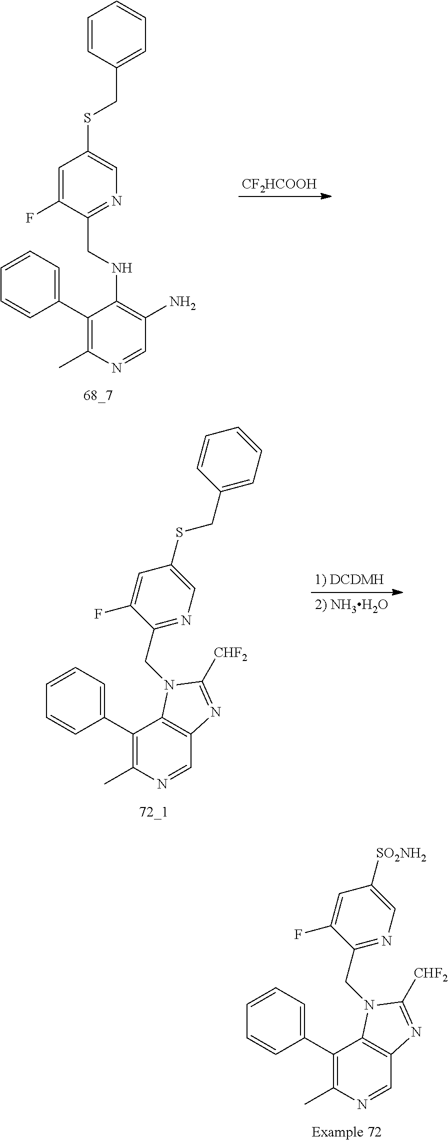 [SMILES: Cc1ncc(N)c(NCc2ncc(SCc3ccccc3)cc2F)c1-c1ccccc1 ; "CF2HCOOH" ; Cc1ncc2nc(C(F)F)n(Cc3ncc(SCc4ccccc4)cc3F)c2c1-c1ccccc1 ; Cc1ncc2nc(C(F)F)n(Cc3ncc(S(N)(=O)=O)cc3F)c2c1-c1ccccc1]

### Step 1: General procedure for preparation of 1-((5-(benzylthio)-3-fluoropyridin-2-yl)methyl)-2-(difluoromethyl)-6-methyl-7-phenyl-1H-imidazo[4,5-c]pyridine (Compound 72_1)

A solution of Compound 68_7 (150 mg, 0.35 mmol) in difluoroacetic acid (5 mL) was stirred at 100° C. for 3 h. The solvent was removed under reduced pressure. The residue was purified by flash silica gel chromatography to afford the corresponding Compound 72_1 (60 mg, 35.1% yield) as a white solid. LCMS: [M+H]⁺=491.1.

### Step 2: General procedure for preparation of 6-((2-(difluoromethyl)-6-methyl-7-phenyl-1H-imidazo[4,5-c]pyridin-1-yl)methyl)-5-fluoropyridine-3-sulfonamide

To a solution of Compound 72_1 (60 mg, 0.12 mmol) in acetonitrile (10 mL)/AcOH(0.3 mL)/H₂O (0.2 mL) at 0° C. was dropwised added a solution of 1,3-dichloro-5,5-dimethylhydantoin/acetonitrile (59 mg/2 mL, 0.30 mmol). The reaction mixture was stirred under 0° C. for 30 min. The reaction mixture was quenched with water and the aqueous layer was extracted with DCM (20 mL×2). The combined organic layer was concentrated under reduced pressure. The residue was dissolved in acetonitrile (5 mL) and the solution was cooled to 0° C. Ammonium hydroxide (0.5 mL) was added and the reaction mixture was stirred at 0° C. for 3 min. The reaction mixture was concentrated under reduced pressure. The residue was purified by Prep-HPLC to afford the corresponding example 72 (8 mg, 14.6% yield). LCMS: [M+H]⁺=448.1. ¹H NMR: (400 MHZ, DMSO-d₆) δ 9.03 (s, 1H), 8.48 (s, 1H), 7.82 (d, J=8.8 Hz, 1H), 7.71 (s, 2H), 7.39 (t, J=51.6 Hz, 1H), 7.36 (t, J=7.4 Hz, 1H), 7.23 (t, J=7.6 Hz, 2H), 6.92 (d, J=7.0 Hz, 2H), 5.31 (s, 2H), 2.19 (s, 3H).

### Example 73: Synthesis of 5-fluoro-6-((6-methoxy-2-methyl-7-phenyl-1H-imidazo[4,5-c]pyridin-1-yl)methyl)pyridine-3-sulfonamide

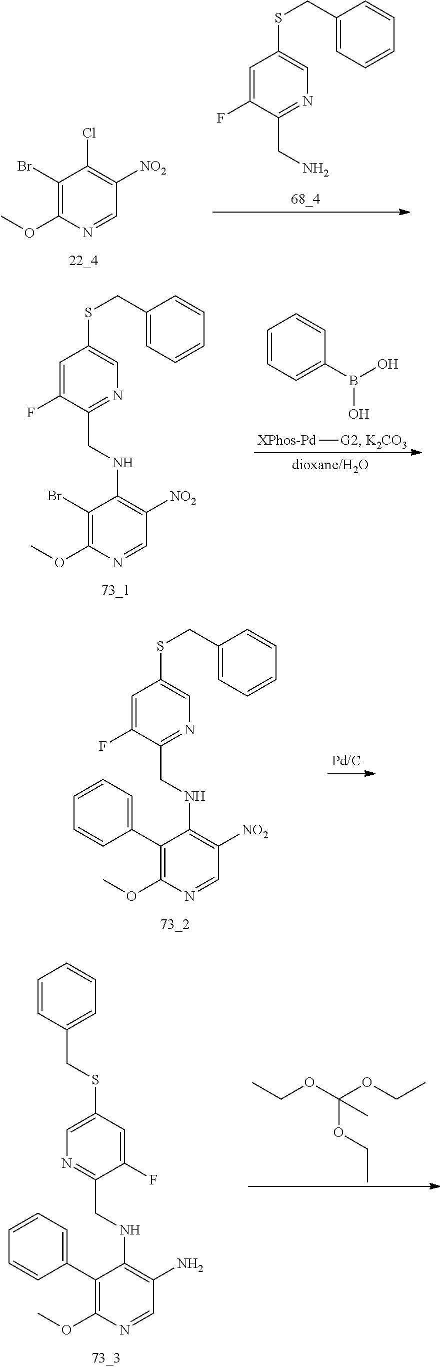

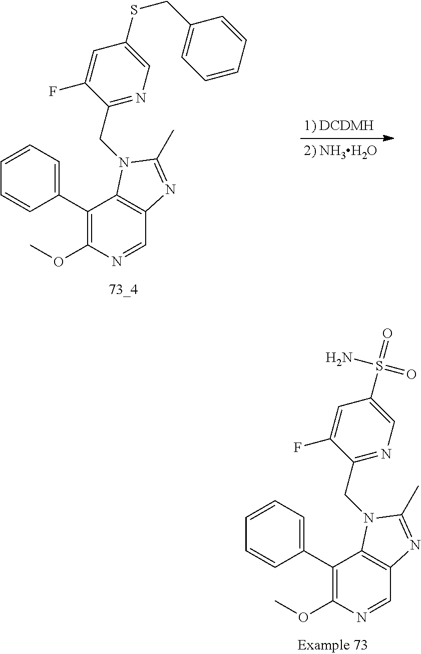 [SMILES: COc1ncc2nc(C)n(Cc3ncc(SCc4ccccc4)cc3F)c2c1-c1ccccc1 ; COc1ncc2nc(C)n(Cc3ncc(S(N)(=O)=O)cc3F)c2c1-c1ccccc1]

### Step 1: General procedure for preparation of N-((5-(benzylthio)-3-fluoropyridin-2-yl)methyl)-3-bromo-2-methoxy-5-nitropyridin-4-amine (Compound 73_1)

To a solution of Compound 22_4 (500 mg, 1.87 mmol) in acetonitrile (10 mL) were added Compound 68_4 (464 mg, 1.87 mmol) and DIPEA (724 mg, 5.61 mmol). This reaction mixture was stirred at 60° C. for 4 h. The solvent was removed under reduced pressure. The obtained residue was purified by flash silica gel chromatography to provide the corresponding Compound 73_1 (620 mg, 69.2% yield) as a yellow solid. LCMS: [M+H]⁺=479.0.

### Step 2: General procedure for preparation of N-((5-(benzylthio)-3-fluoropyridin-2-yl)methyl)-2-methoxy-5-nitro-3-phenylpyridin-4-amine (Compound 73_2)

To a solution of Compound 73_1 (620 mg, 1.29 mmol) in dioxane/H₂O (10 mL/1 mL) were added phenylboronic acid (237 mg, 1.94 mmol), XPhos-Pd-G2 (103 mg, 0.13 mmol) and K₂CO₃ (534 mg, 3.87 mmol). The reaction mixture was stirred under 100° C. for 16 h under N₂ atmosphere. The solvent was removed under reduced pressure. The obtained residue was purified by flash silica gel chromatography to provide the corresponding Compound 73_2 (540 mg, 87.6% yield) as a yellow solid. LCMS: [M+H]⁺=477.1.

### Step 3: General procedure for preparation of N₄-((5-(benzylthio)-3-fluoropyridin-2-yl)methyl)-6-methoxy-5-phenylpyridine-3,4-diamine (Compound 73_3)

To a solution of Compound 73_2 (540 mg, 1.13 mmol) in MeOH(6 mL)/DCM (6 mL) was added Pd/C (100 mg, 10% wt). The reaction mixture was stirred at room temperature for 16 h under H₂ atmosphere. The reaction solution was filtered and the filter cake was washed with MeOH(10 mL×3). The filtrate was concentrated under reduced pressure. The obtained residue was purified by flash silica gel chromatography to provide the corresponding Compound 73_3 (450 mg, 88.9% yield) as a light yellow solid. LCMS: [M+H]⁺=447.1.

### Step 4: General procedure for preparation of 1-((5-(benzylthio)-3-fluoropyridin-2-yl)methyl)-6-methoxy-2-methyl-7-phenyl-1H-imidazo[4,5-c]pyridine (Compound 73_4)

To a solution of Compound 73_3 (450 mg, 1.01 mmol) in EtOH(10 mL) were added triethyl orthoacetate (1.63 g, 10.1 mmol) and Py-HCl (12 mg, 0.10 mmol). The reaction mixture was stirred at 100° C. for 1 h. The solvent was removed under reduced pressure. The obtained residue was purified by flash silica gel chromatography to provide the corresponding Compound 73_4 (420 mg, 88.6% yield) as a yellow solid. LCMS: [M+H]⁺=471.2.

### Step 5: General procedure for preparation of 5-fluoro-6-((6-methoxy-2-methyl-7-phenyl-1H-imidazo[4,5-c]pyridin-1-yl)methyl)pyridine-3-sulfonamide

To a solution of Compound 73_4 (420 mg, 0.89 mmol) in acetonitrile (10 mL)/AcOH(0.3 mL)/H₂O (0.2 mL) at 0° C. was added drop wise a solution of 1,3-dichloro-5,5-dimethylhydantoin/acetonitrile (438 mg/3 mL, 0.30 mmol). The mixture was stirred under 0° C. for 30 min. The reaction mixture was quenched with water and the aqueous layer was extracted with DCM (10 mL×3). The combined organic layer was concentrated under reduced pressure. The residue was dissolved in acetonitrile (5 mL) and the solution was cooled to 0° C. Ammonium hydroxide (3 mL) was added drop wise and the reaction solution was stirred at 0° C. for 3 min. The reaction mixture was concentrated under reduced pressure. The obtained residue was purified by Prep-HPLC to afford the corresponding example 73 (180 mg, 47.2% yield). LCMS: [M+H]⁺=428.3. ¹H NMR: (400 MHZ, DMSO-d₆) δ 8.48 (s, 1H), 8.47 (s, 1H), 7.77 (d, J=9.2 Hz, 1H), 7.70 (s, 2H), 7.29 (t, J=7.2 Hz, 1H), 7.17 (t, J=7.6 Hz, 2H), 6.85 (d, J=7.6 Hz, 2H), 5.15 (s, 2H), 3.73 (s, 3H), 2.44 (s, 3H).

### Example 74: Synthesis of 6-((6-methoxy-2-methyl-7-phenyl-1H-imidazo[4,5-c]pyridin-1-yl)methyl)pyridine-3-sulfonamide

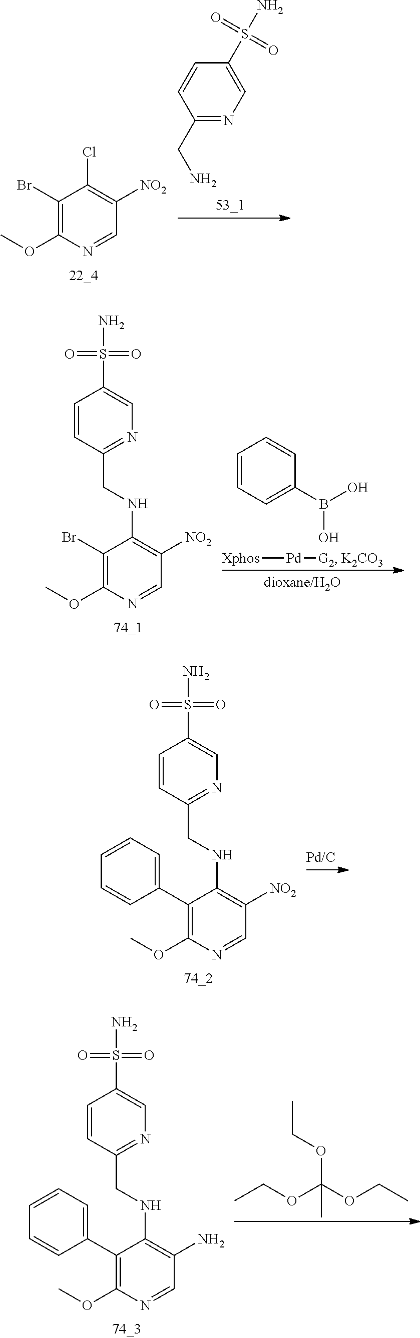

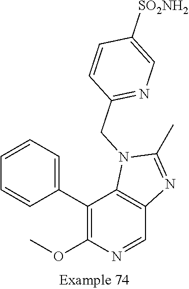 [SMILES: COc1ncc2nc(C)n(Cc3ccc(S(N)(=O)=O)cn3)c2c1-c1ccccc1]

### Step 1: General procedure for preparation of 4-(((3-bromo-2-methoxy-5-nitropyridin-4-yl)amino) methyl)benzenesulfonamide (Compound 74_1)

To a solution of Compound 22_4 (500 mg, 1.87 mmol) in acetonitrile (10 mL) were added Compound 53_1 (350 mg, 1.87 mmol) and DIPEA (724 mg, 5.61 mmol). The reaction mixture was stirred at 60° C. for 2 h. After the solvent was removed under reduced pressure, the obtained residue was purified by flash silica gel chromatography to provide the corresponding Compound 74_1 (620 mg, 79.3% yield) as a yellow solid. LCMS: [M+H]⁺=418.0.

### Step 4: General procedure for preparation of 6-(((2-methoxy-5-nitro-3-phenylpyridin-4-yl)amino) methyl)pyridine-3-sulfonamide (Compound 74_2)

To a solution of Compound 74_1 (620 mg, 1.48 mmol) in dioxane/H₂O (10 mL/1 mL) were added phenylboronic acid (271 mg, 2.22 mmol), XPhos-Pd-G2 (118 mg, 0.15 mmol) and K₂CO₃ (613 mg, 4.44 mmol). The mixture was stirred at 100° C. for 16 h under N₂ atmosphere. After the solvent was removed under reduced pressure, the obtained residue was purified by flash silica gel chromatography to provide the corresponding Compound 74_2 (520 mg, 84.4% yield) as a yellow solid. LCMS: [M+H]⁺=416.1.

### Step 5: General procedure for preparation of 6-(((5-amino-2-methoxy-3-phenylpyridin-4-yl)amino) methyl)pyridine-3-sulfonamide (Compound 74_3)

To a solution of Compound 74_2 (520 mg, 1.25 mmol) in MeOH(6 mL)/DCM (6 mL) was added Pd/C (100 mg, 10% wt). The reaction mixture was stirred at room temperature for 16 h under H₂ atmosphere. The reaction solution was filtered and the filter cake was washed with MeOH(10 mL×3). The filtrate was concentrated under reduced pressure. The obtained residue was purified by flash silica gel chromatography to provide the corresponding Compound 74_3 (440 mg, 91.3% yield) as a light yellow solid. LCMS: [M+H]⁺=386.1.

### Step 3: General procedure for preparation of 6-((6-methoxy-2-methyl-7-phenyl-1H-imidazo[4,5-c]pyridin-1-yl)methyl)pyridine-3-sulfonamide

To a solution of Compound 74_3 (100 mg, 0.26 mmol) in EtOH(5 mL) were added 1,1,1-triethoxyethane (421 mg, 2.59 mmol) and pyridine hydrochloride (3 mg, 0.026 mmol). The reaction mixture was stirred at 80° C. for 1 h. The solvent was removed under reduced pressure. The obtained residue was purified by Prep-HPLC to afford the corresponding example 74 (15 mg, 14.1%). LCMS: [M+H]⁺=410.2. ¹H NMR: (400 MHZ, DMSO-d₆) δ 8.65 (d, J=1.8 Hz, 1H), 8.49 (s, 1H), 7.94 (dd, J=8.3, 2.3 Hz, 1H), 7.56 (s, 2H), 7.29 (t, J=7.4 Hz, 1H), 7.19 (t, J=7.5 Hz, 2H), 6.96 (d, J=6.9 Hz, 2H), 6.63 (d, J=8.4 Hz, 1H), 5.03 (s, 2H), 3.74 (s, 3H), 2.41 (s, 3H).

### Example 75: Synthesis of 6-((2-(difluoromethyl)-6-methoxy-7-phenyl-1H-imidazo[4,5-c]pyridin-1-yl)methyl)pyridine-3-sulfonamide

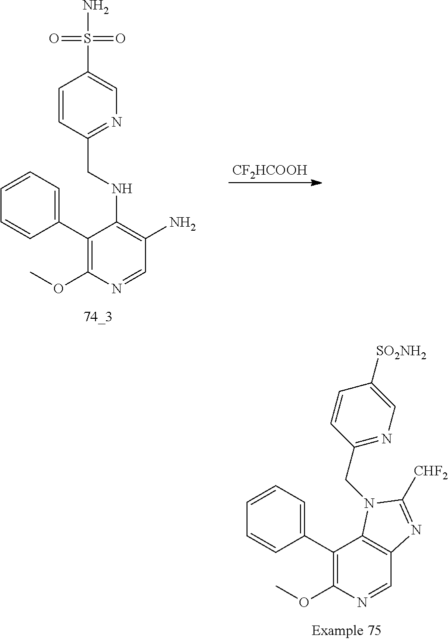 [SMILES: COc1ncc(N)c(NCc2ccc(S(N)(=O)=O)cn2)c1-c1ccccc1 ; "CF2HCOOH" ; COc1ncc2nc(C(F)F)n(Cc3ccc(S(N)(=O)=O)cn3)c2c1-c1ccccc1]

A solution of Compound 74_3 (80 mg, 0.21 mmol) in trifluoroacetic acid (5 mL) was stirred at 100° C. for 3 h. The solvent was removed under reduced pressure. The obtained residue was purified by was purified by Prep-HPLC to afford the corresponding example 75 (12 mg, 13.0%). LCMS: [M+H]=446.2. ¹H NMR: (400 MHZ, DMSO-d₆) δ 8.81 (s, 1H), 8.59 (d, J=2.4 Hz, 1H), 7.99-7.88 (m, 1H), 7.56 (s, 2H), 7.38 (t, J=51.6 Hz, 1H), 7.28 (t, J=7.6 Hz, 1H), 7.17 (t, J=7.6 Hz, 2H), 6.95 (d, J=7.2 Hz, 2H), 6.68 (d, J=8.3 Hz, 1H), 5.22 (s, 2H), 3.79 (s, 3H).

### Example 76: Synthesis of 6-((2-(difluoromethyl)-6-methoxy-7-phenyl-1H-imidazo[4,5-c]pyridin-1-yl)methyl)-5-fluoropyridine-3-sulfonamide

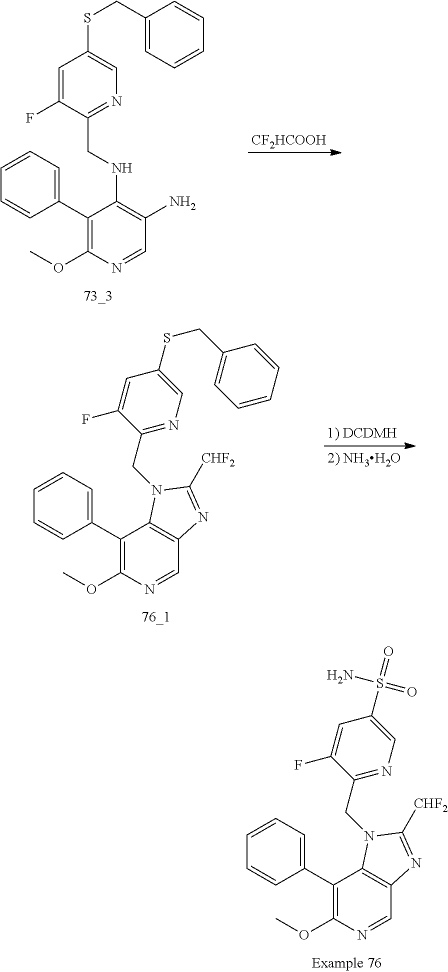

### Step 1: General procedure for preparation of 1-((5-(benzylthio)-3-fluoropyridin-2-yl)methyl)-2-(difluoromethyl)-6-methoxy-7-phenyl-1H-imidazo[4,5-c]pyridine (Compound 76_1)

A solution of Compound 73_3 (100 mg, 0.22 mmol) in 2,2-difluoroacetic acid (3 mL) was stirred at 100° C. for 3 h. The solvent was removed under reduced pressure. The obtained residue was purified by flash silica gel chromatography to provide the corresponding Compound 76_1 (73 mg, 64.3%) as a light yellow solid. LCMS: [M+H]⁺=507.0.

### Step 2: General procedure for preparation of 6-((2-(difluoromethyl)-6-methoxy-7-phenyl-1H-imidazo[4,5-c]pyridin-1-yl)methyl)-5-fluoropyridine-3-sulfonamide

To a solution of Compound 76_1 (73 mg, 0.14 mmol) in acetonitrile (5 mL)/AcOH(0.3 mL)/H₂O (0.2 mL) at 0° C. was added dropwise a solution of 1,3-dichloro-5,5-dimethylhydantoin/acetonitrile (69 mg/l mL, 0.30 mmol). The mixture was stirred under 0° C. for 30 min. The reaction mixture was quenched with water and the aqueous layer was extracted with DCM (10 mL×3). The combined organic layer was concentrated under reduced pressure. The obtained residue was dissolved in acetonitrile (5 mL) and the solution was cooled to 0° C. Ammonium hydroxide (0.5 mL) was added drop wise and the reaction solution was stirred at 0° C. for 3 min. The reaction mixture was concentrated under reduced pressure. The obtained residue was purified by Prep-HPLC to afford the corresponding example 76 (10 mg, 15.0% yield). LCMS: [M+H]⁺=416.1. ¹H NMR: (400 MHz, DMSO-d₆) δ 8.80 (s, 1H), 8.46 (s, 1H), 7.77 (dd, J=9.1, 1.7 Hz, 1H), 7.72 (s, 2H), 7.37 (t, J=51.6 Hz, 1H), 7.29 (t, J=7.6 Hz, 1H), 7.18 (t, J=7.5 Hz, 2H), 6.91 (d, J=6.8 Hz, 2H), 5.33 (s, 2H), 3.78 (s, 3H).

### Example 77: Synthesis of 6-((6-methoxy-2-methyl-7-phenyl-1H-imidazo[4,5-c]pyridin-1-yl)methyl)pyridine-3-sulfonamide

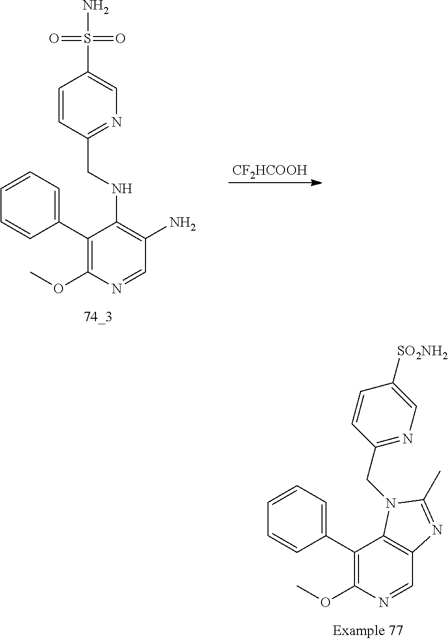 [SMILES: COc1ncc(N)c(NCc2ccc(S(N)(=O)=O)cn2)c1-c1ccccc1 ; "CF2HCOOH" ; COc1ncc2nc(C)n(Cc3ccc(S(N)(=O)=O)cn3)c2c1-c1ccccc1]

A solution of Compound 74_3 (80 mg, 0.21 mmol) in trifluoroacetic acid (5 mL) was stirred at 100° C. for 3 h. The solvent was removed under reduced pressure. The obtained residue was purified by was purified by Prep-HPLC to afford the corresponding example 77 (13 mg, 15.3%). LCMS: [M+H]⁺=464.2. ¹H NMR: (400 MHz, DMSO-d₆) δ 8.90 (s, 1H), 8.60 (d, J=1.9 Hz, 1H), 7.93 (dd, J=8.2, 2.2 Hz, 1H), 7.56 (s, 2H), 7.29 (t, J=7.5 Hz, 1H), 7.17 (t, J=7.5 Hz, 2H), 6.97 (d, J=6.3 Hz, 2H), 6.80 (d, J=8.4 Hz, 1H), 5.24 (s, 2H), 3.80 (s, 3H).

### Example 78: Synthesis of 5-fluoro-6-((6-methoxy-7-phenyl-2-(trifluoromethyl)-1H-imidazo[4,5-c]pyridin-1-yl)methyl)pyridine-3-sulfonamide

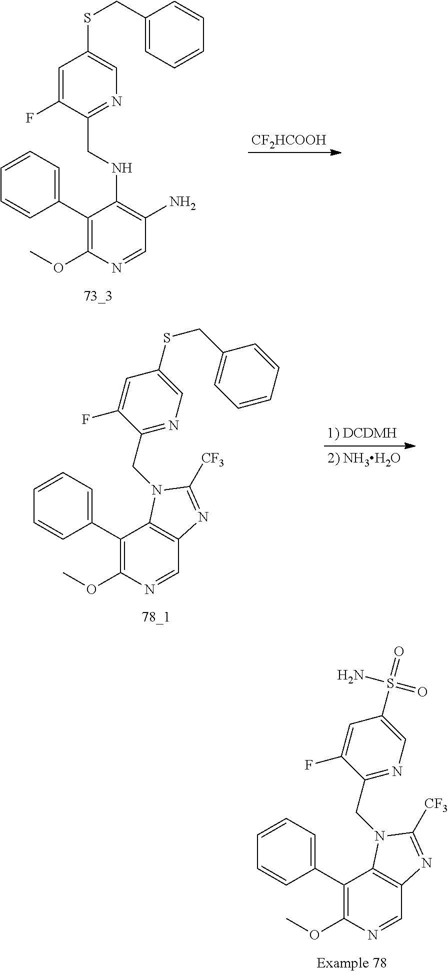

### Step 1: General procedure for preparation of 1-((5-(benzylthio)-3-fluoropyridin-2-yl)methyl)-6-methoxy-7-phenyl-2-(trifluoromethyl)-1H-imidazo[4,5-c]pyridine (Compound 78_1)

A solution of Compound 73_3 (100 mg, 0.22 mmol) in trifluoroacetic acid (3 mL) was stirred at 100° C. for 3 h. The solvent was removed under reduced pressure. The obtained residue was purified by flash silica gel chromatography to provide the corresponding Compound 78_1 (80 mg, 68.1%) as a light yellow solid. LCMS: [M+H]⁺=525.1.

### Step 2: General procedure for preparation of 5-fluoro-6-((6-methoxy-7-phenyl-2-(trifluoromethyl)-1H-imidazo[4,5-c]pyridin-1-yl)methyl)pyridine-3-sulfonamide

To a solution of Compound 78_1 (80 mg, 0.15 mmol) in acetonitrile (5 mL)/AcOH(0.3 mL)/H₂O (0.2 mL) at 0° C. was added drop wise a solution of 1,3-dichloro-5,5-dimethylhydantoin/acetonitrile (74 mg/l mL, 0.38 mmol). The mixture was stirred under 0° C. for 30 min. The reaction mixture was quenched with water and the aqueous layer was extracted with DCM (10 mL×3). The organic layer was concentrated under reduced pressure. The residue was dissolved in acetonitrile (5 mL) and the solution was cooled to 0° C. Ammonium hydroxide (0.5 mL) was added and the reaction mixture was stirred at 0° C. for 3 min. The reaction mixture was concentrated under reduced pressure and the obtained residue was purified by Prep-HPLC to afford the corresponding example 78 (18 mg, 24.5% yield). LCMS: [M+H]⁺=482.2. ¹H NMR: (400 MHZ, DMSO-d₆) δ 8.89 (s, 1H), 8.47 (s, 1H), 7.80-7.63 (m, 3H), 7.32-7.28 (m, 1H), 7.24-7.14 (m, 2H), 6.91 (s, 2H), 5.35 (s, 2H), 3.79 (s, 3H).

### Example 79: Synthesis of 6-((3,6-dimethyl-2-oxo-7-phenyl-2,3-dihydro-1H-imidazo[4,5-c]pyridin-1-yl)methyl)-5-fluoropyridine-3-sulfonamide

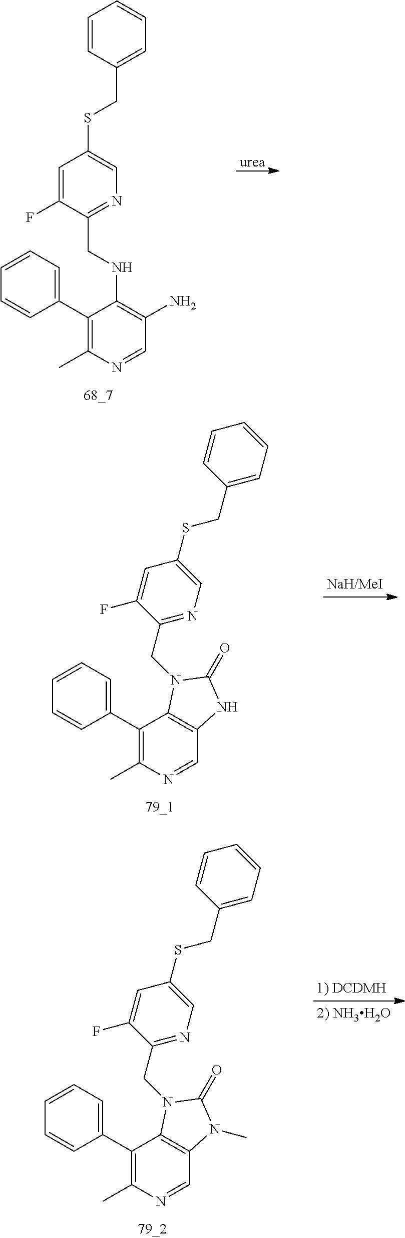 [SMILES: Cc1ncc(N)c(NCc2ncc(SCc3ccccc3)cc2F)c1-c1ccccc1 ; "urea" ; Cc1ncc2[nH]c(=O)n(Cc3ncc(SCc4ccccc4)cc3F)c2c1-c1ccccc1 ; Cc1ncc2c(c1-c1ccccc1)n(Cc1ncc(SCc3ccccc3)cc1F)c(=O)n2C]

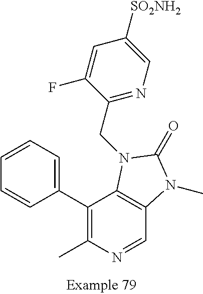 [SMILES: Cc1ncc2c(c1-c1ccccc1)n(Cc1ncc(S(N)(=O)=O)cc1F)c(=O)n2C]

### Step 1: General procedure for preparation of 1-((5-(benzylthio)-3-fluoropyridin-2-yl)methyl)-6-methyl-7-phenyl-1,3-dihydro-2H-imidazo[4,5-c]pyridin-2-one (Compound 79_1)

A mixture of Compound 68_7 (200 mg, 0.46 mmol) in urea (552 mg, 9.2 mmol) was stirred at 150° C. for 3 h. Then the reaction mixture was diluted with water (10 mL) and stirred for another 1 h at room temperature. The precipitate is filtered and the filter cake was dried to give the crude product Compound 79_1 (100 mg, 47.2% yield) as a yellow solid. LCMS: [M+H]⁺=457.1.

### Step 2: General procedure for preparation of 1-((5-(benzylthio)-3-fluoropyridin-2-yl)methyl)-3,6-dimethyl-7-phenyl-1,3-dihydro-2H-imidazo[4,5-c]pyridin-2-one (Compound 79_2)

To a solution of Compound 79_1 (100 mg, 0.22 mmol) in DMF (5 mL) was added NaH(18 mg, 0.44 mmol, 60% in oil) under an ice bath. The mixture was stirred at this temperature for 30 min and methyl iodide (62 mg, 0.44 mmol) was added. The reaction mixture was stirred at room temperature for 2 h. Then the reaction mixture was diluted with water (10 mL). The aqueous phase was extracted with ethyl acetate (10 mL×2). The combined organic phase was washed by brine (10 mL), dried over anhydrous sodium sulfate, filtered, and concentrated in vacuum. The obtained residue was purified by flash silica gel chromatography to afford the corresponding Compound 79_2 (60 mg, 58.2% yield) as a white solid. LCMS: [M+H]⁺=471.2.

### Step 3: General procedure for preparation of 6-((3,6-dimethyl-2-oxo-7-phenyl-2,3-dihydro-1H-imidazo[4,5-c]pyridin-1-yl)methyl)-5-fluoropyridine-3-sulfonamide

To a solution of Compound 79_2 (60 mg, 0.13 mmol) in acetonitrile (5 mL)/AcOH(0.3 mL)/H₂O (0.2 mL) at 0° C. was added drop wise a solution of 1,3-dichloro-5,5-dimethylhydantoin/acetonitrile (64 mg/l mL, 0.33 mmol). The mixture was stirred under 0° C. for 30 min. The reaction mixture was quenched with water and the aqueous layer was extracted with DCM (10 mL×3). The organic layer was concentrated under reduced pressure. The residue was dissolved in acetonitrile (5 mL) and the solution was cooled to 0° C. Ammonium hydroxide (0.5 mL) was added and the reaction solution was stirred at 0° C. for 3 min. The reaction mixture was concentrated under reduced pressure, The obtained residue was purified by Prep-HPLC to afford the corresponding example 79 (8 mg, 14.7% yield). LCMS: [M+H]⁺=428.2. ¹H NMR: (400 MHZ, DMSO-d₆) δ 8.52 (s, 1H), 8.35 (s, 1H), 7.79-7.64 (m, 3H), 7.30 (t, J=7.4 Hz, 1H), 7.18 (t, J=7.6 Hz, 2H), 6.86 (d, J=7.5 Hz, 2H), 4.80 (s, 2H), 3.48 (s, 3H), 2.06 (s, 3H).

### Example 80: Synthesis of 5-fluoro-6-((6-methoxy-3-methyl-2-oxo-7-phenyl-2,3-dihydro-1H-imidazo[4,5-c]pyridin-1-yl)methyl)pyridine-3-sulfonamide

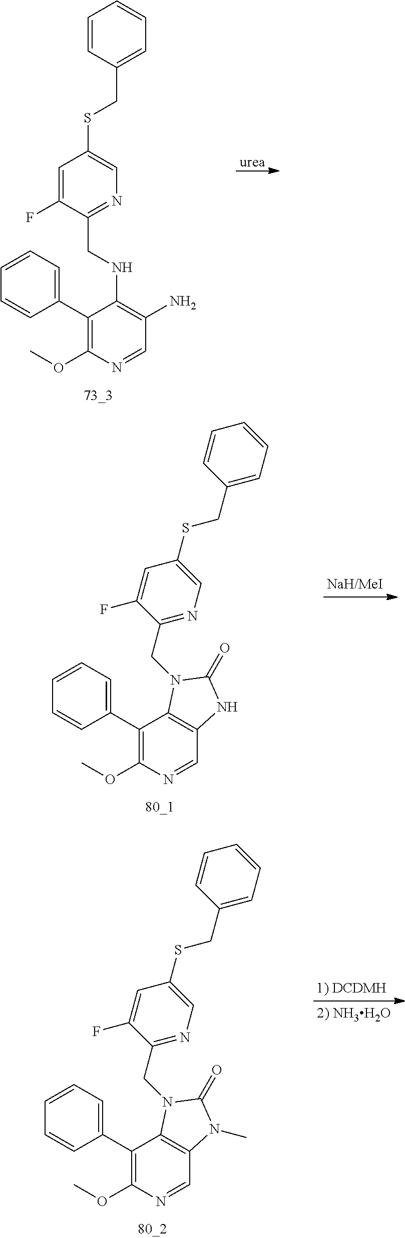 [SMILES: COc1ncc(N)c(NCc2ncc(SCc3ccccc3)cc2F)c1-c1ccccc1 ; "urea" ; COc1ncc2[nH]c(=O)n(Cc3ncc(SCc4ccccc4)cc3F)c2c1-c1ccccc1 ; COc1ncc2c(c1-c1ccccc1)n(Cc1ncc(SCc3ccccc3)cc1F)c(=O)n2C]

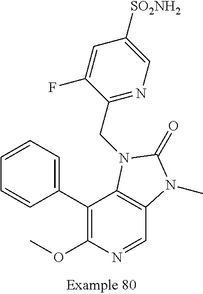 [SMILES: COc1ncc2c(c1-c1ccccc1)n(Cc1ncc(S(N)(=O)=O)cc1F)c(=O)n2C]

### Step 1: General procedure for preparation of 1-((5-(benzylthio)-3-fluoropyridin-2-yl)methyl)-6-methoxy-7-phenyl-1,3-dihydro-2H-imidazo[4,5-c]pyridin-2-one (Compound 80_1)

A mixture of Compound 73_3 (200 mg, 0.45 mmol) in urea (552 mg, 9.2 mmol) was stirred at 150° C. for 3 h. Then the reaction mixture was diluted with water (10 mL) and stirred for 1 h at room temperature. The precipitate is filtered off and the filter cake was dried to give the crude product Compound 80_1 (90 mg, 42.5% yield) as a yellow solid. LCMS: [M+H]⁺=473.1.

### Step 2: General procedure for preparation of 1-((5-(benzylthio)-3-fluoropyridin-2-yl)methyl)-6-methoxy-3-methyl-7-phenyl-1,3-dihydro-2H-imidazo[4,5-c]pyridin-2-one (Compound 80_2)

To a solution of Compound 80_1 (90 mg, 0.19 mmol) in DMF (5 mL) were added NaH(15 mg, 0.38 mmol, 60% in oil) under an ice bath. The mixture was stirred at this temperature for 30 min and methyl iodide (62 mg, 0.44 mmol) was added. The reaction mixture was stirred at room temperature for 2 h. Then the reaction mixture was diluted with water (10 mL). The aqueous phase was extracted with ethyl acetate (10 mL×2). The combined organic phase was washed by brine (10 mL), dried over anhydrous sodium sulfate, filtered, and concentrated in vacuum. The obtained residue was purified by flash silica gel chromatography to afford the corresponding Compound 80_2 (55 mg, 59.4% yield) as a white solid. LCMS: [M+H]⁺=487.2.

### Step 3: General procedure for preparation of 5-fluoro-6-((6-methoxy-3-methyl-2-oxo-7-phenyl-2,3-dihydro-1H-imidazo[4,5-c]pyridin-1-yl)methyl)pyridine-3-sulfonamide

To a solution of Compound 80_2 (55 mg, 0.11 mmol) in acetonitrile (5 mL)/AcOH(0.3 mL)/H₂O (0.2 mL) at 0° C. was added drop wise a solution of 1,3-dichloro-5,5-dimethylhydantoin/acetonitrile (43 mg/l mL, 0.22 mmol). The mixture was stirred under 0° C. for 30 min. The reaction mixture was quenched with water and the aqueous layer was extracted with DCM. The combined organic layer was concentrated under reduced pressure. The residue was dissolved in acetonitrile (5 mL) and the mixture was cooled to 0° C. Ammonium hydroxide (0.5 mL) was added and the reaction solution was stirred at 0° C. for 3 min. The reaction mixture was concentrated under reduced pressure. The obtained residue was purified by Prep-HPLC to afford the corresponding example 80 (9 mg, 18.0% yield). LCMS: [M+H]⁺=444.2. ¹H NMR: (400 MHZ, DMSO-d₆) δ 8.49 (s, 1H), 8.01 (s, 1H), 7.78-7.68 (m, 3H), 7.24 (t, J=7.4 Hz, 1H), 7.13 (t, J=7.6 Hz, 2H), 6.84 (d, J=7.1 Hz, 2H), 4.86 (s, 2H), 3.68 (s, 3H), 3.44 (s, 3H).

### Example 81: Synthesis of 6-((3,6-dimethyl-2-oxo-7-phenyl-2,3-dihydro-1H-imidazo[4,5-c]pyridin-1-yl)methyl)pyridine-3-sulfonamide

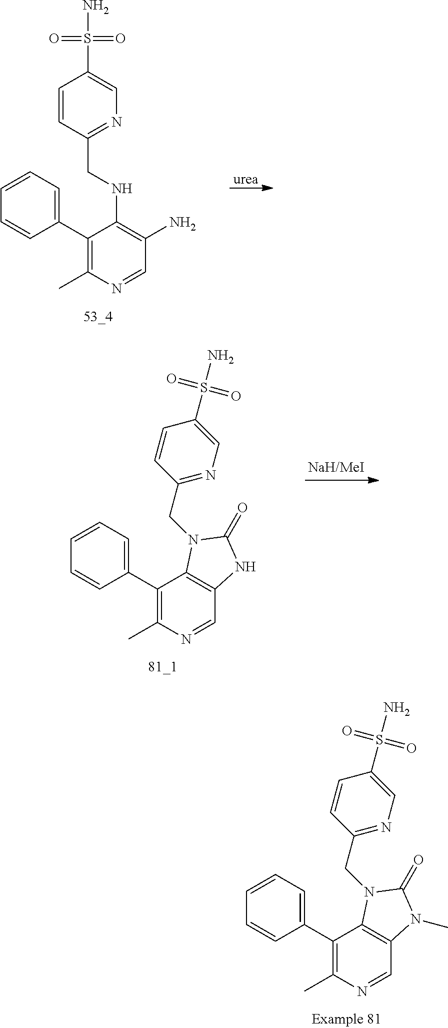 [SMILES: Cc1ncc(N)c(NCc2ccc(S(N)(=O)=O)cn2)c1-c1ccccc1 ; "urea" ; Cc1ncc2[nH]c(=O)n(Cc3ccc(S(N)(=O)=O)cn3)c2c1-c1ccccc1 ; Cc1ncc2c(c1-c1ccccc1)n(Cc1ccc(S(N)(=O)=O)cn1)c(=O)n2C]

### Step 1: General procedure for preparation of 6-((6-methyl-2-oxo-7-phenyl-2,3-dihydro-1H-imidazo[4,5-c]pyridin-1-yl)methyl)pyridine-3-sulfonamide (Compound 81_1)

A mixture of Compound 53_4 (200 mg, 0.54 mmol) in urea (648 mg, 10.8 mmol) was stirred at 150° C. for 3 h. Then the reaction mixture was diluted with water (10 mL) and stirred for 1 h at room temperature. The precipitate is filtered off and the filter cake was dried to give the crude product Compound 81_1 (80 mg, 37.4% yield) as a yellow solid. LCMS: [M+H]⁺=396.1.

### Step 2: General procedure for preparation of 6-((3,6-dimethyl-2-oxo-7-phenyl-2,3-dihydro-1H-imidazo[4,5-c]pyridin-1-yl)methyl)pyridine-3-sulfonamide

To a solution of Compound 81_1 (80 mg, 0.20 mmol) in DMF (5 mL) were added NaH(16 mg, 0.40 mmol, 60% in oil) under an ice bath. The mixture was stirred at this temperature for 30 min and methyl iodide (62 mg, 0.44 mmol) was added. The reaction mixture was stirred at room temperature for 2 h. Then the reaction mixture was diluted with water (10 mL). The aqueous phase was extracted with ethyl acetate (10 mL×2). The combined organic phase was washed by brine (10 mL), dried over anhydrous sodium sulfate, filtered, and concentrated in vacuum. The obtained residue was purified by Prep-HPLC to afford the corresponding example 81 (9 mg, 10.9% yield). LCMS: [M+H]⁺=410.2. ¹H NMR: (400 MHZ, DMSO-d₆) δ 8.63 (d, J=2.1 Hz, 1H), 8.36 (s, 1H), 7.92 (dd, J=8.3, 2.3 Hz, 1H), 7.54 (s, 2H), 7.28 (t, J=7.5 Hz, 1H), 7.17 (t, J=7.5 Hz, 2H), 6.91 (d, J=7.0 Hz, 2H), 6.85 (d, J=8.2 Hz, 1H), 4.67 (s, 2H), 3.47 (s, 3H), 2.08 (s, 3H).

### Example 82: Synthesis of 6-((6-methoxy-3-methyl-2-oxo-7-phenyl-2,3-dihydro-1H-imidazo[4,5-c]pyridin-1-yl)methyl)pyridine-3-sulfonamide

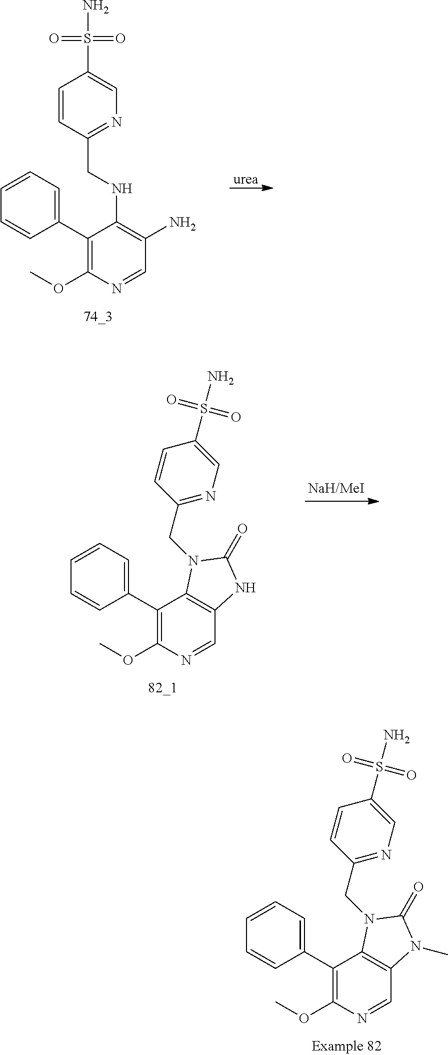 [SMILES: COc1ncc(N)c(NCc2ccc(S(N)(=O)=O)cn2)c1-c1ccccc1 ; "urea" ; COc1ncc2[nH]c(=O)n(Cc3ccc(S(N)(=O)=O)cn3)c2c1-c1ccccc1 ; COc1ncc2c(c1-c1ccccc1)n(Cc1ccc(S(N)(=O)=O)cn1)c(=O)n2C]

### Step 1: General procedure for preparation of 6-((6-methoxy-2-oxo-7-phenyl-2,3-dihydro-1H-imidazo[4,5-c]pyridin-1-yl)methyl)pyridine-3-sulfonamide (Compound 82_1)

A mixture of Compound 74_3 (200 mg, 0.52 mmol) in urea (624 mg, 10.4 mmol) was stirred at 150° C. for 3 h. Then the reaction mixture was diluted with water (10 mL) and stirred for 1 h at room temperature. The precipitate is filtered off and the filter cake was dried to give the crude product Compound 82_1 (110 mg, 51.4% yield) as a yellow solid. LCMS: [M+H]⁺=4121.

### Step 2: General procedure for preparation of 6-((6-methoxy-3-methyl-2-oxo-7-phenyl-2,3-dihydro-1H-imidazo[4,5-c]pyridin-1-yl)methyl)pyridine-3-sulfonamide

To a solution of Compound 82_1 (110 mg, 0.27 mmol) in DMF (5 mL) were added NaH(22 mg, 0.54 mmol, 60% in oil) under an ice bath, The mixture was stirred at this temperature for 30 min and methyl iodide (77 mg, 0.54 mmol) was added. The reaction mixture was stirred at room temperature for 2 h. Then the reaction mixture was diluted with water (10 mL). The aqueous phase was extracted with ethyl acetate (10 mL×2). The combined organic phase was washed by brine (10 mL), dried over anhydrous sodium sulfate, filtered, and concentrated in vacuum. The obtained residue was purified by Prep-HPLC to afford the corresponding example 82 (23 mg, 20.2% yield). LCMS: [M+H]⁺=426.2. ¹H NMR: (400 MHZ, DMSO-d₆) δ 8.61 (d, J=1.9 Hz, 1H), 8.02 (s, 1H), 7.89 (dd, J=8.2, 2.2 Hz, 1H), 7.56 (s, 2H), 7.24 (t, J=7.4 Hz, 1H), 7.13 (t, J=7.5 Hz, 2H), 6.89 (d, J=7.1 Hz, 2H), 6.80 (d, J=8.3 Hz, 1H), 4.73 (s, 2H), 3.69 (s, 3H), 3.51 (s, 3H).

### Example 83: Synthesis of 5-fluoro-6-((6-hydroxy-2-methyl-7-phenyl-1H-imidazo[4,5-c]pyridin-1-yl)methyl)pyridine-3-sulfonamide

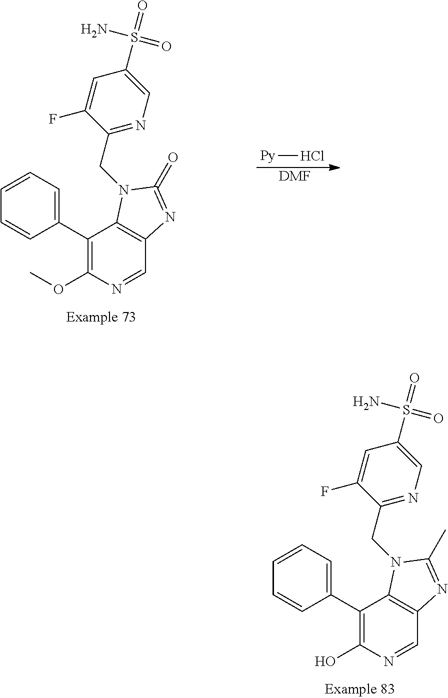

To a solution of example 73 (150 mg, 0.35 mmol) in DMF (5 mL) was added pyridine hydrochloride (257 mg, 3.50 mmol). The reaction mixture was stirred at 100° C. for 16 h. Solvent was removed under reduced pressure. The obtained residue was purified by was purified by Prep-HPLC to afford the corresponding example 83 (75 mg, 51.7% yield). LCMS: [M+H]⁺=414.2. ¹H NMR: (400 MHZ, DMSO-d₆) δ 11.49 (brs, 1H), 8.54 (s, 1H), 7.85 (s, 1H), 7.76 (dd, J=9.1, 1.6 Hz, 1H), 7.72 (s, 2H), 7.23 (t, J=7.4 Hz, 1H), 7.13 (t, J=7.6 Hz, 2H), 6.80 (d, J=7.1 Hz, 2H), 5.04 (s, 2H), 2.35 (s, 3H).

### Example 84: Synthesis of 4-((6-(methoxy-d₃)-2-methyl-7-phenyl-1H-imidazo[4,5-c]pyridin-1-yl)methyl)benzenesulfonamide

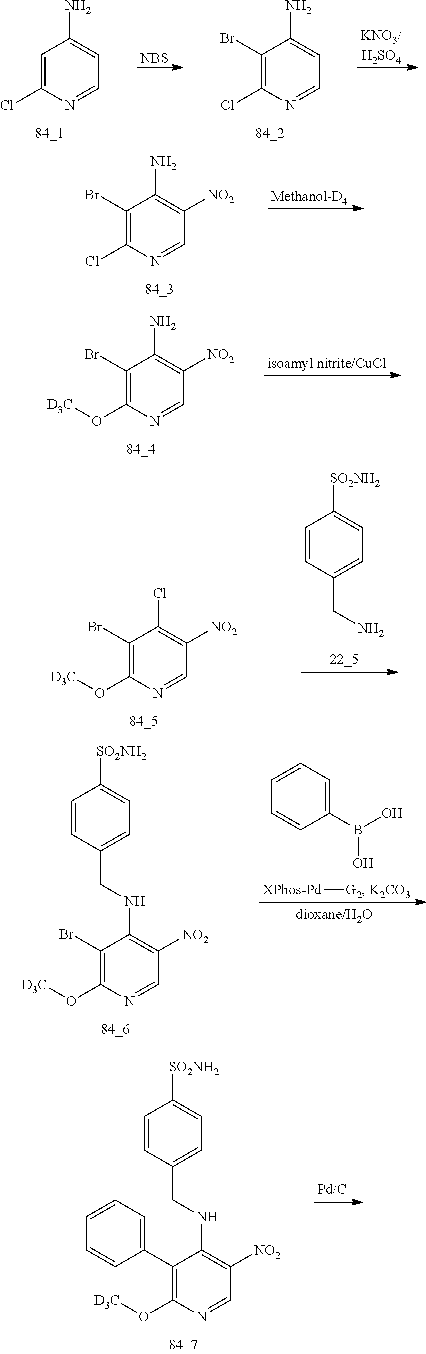

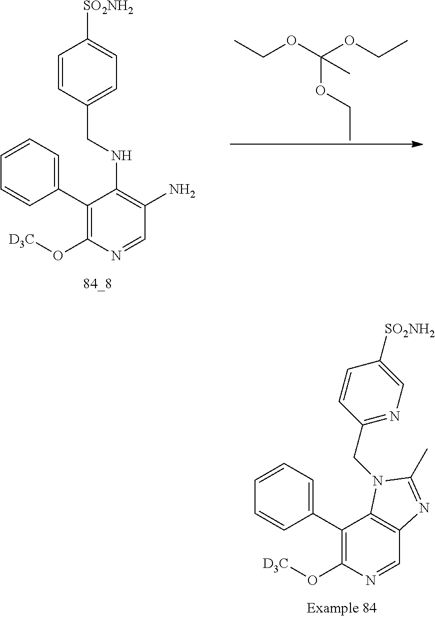 [SMILES: [2H]C([2H])([2H])Oc1ncc(N)c(NCc2ccc(S(N)(=O)=O)cc2)c1-c1ccccc1 ; CCOC(C)(OCC)OCC ; [2H]C([2H])([2H])Oc1ncc2nc(C)n(Cc3ccc(S(N)(=O)=O)cn3)c2c1-c1ccccc1]

### Step 1: General procedure for preparation of 3-bromo-2-chloropyridin-4-amine (Compound 84_2)

To a solution of Compound 84_1 (20 g, 156 mmol) in acetonitrile (120 mL) was added NBS (29 g, 163 mmol). The reaction mixture was stirred at room temperature for 2 h. Then the reaction mixture was diluted with water (80 mL). The aqueous phase was extracted with ethyl acetate (80 mL×3). The combined organic phase was washed by brine (80 mL), dried over anhydrous sodium sulfate, filtered, and concentrated in vacuum. The obtained residue was purified by flash silica gel chromatography to afford the corresponding Compound 84_2 (21 g, 65.1% yield) as a white solid. LCMS: [M+H]⁺=207.0.

### Step 2: General procedure for preparation of 3-bromo-2-chloro-5-nitropyridin-4-amine (Compound 84_3)

A solution of Compound 84_2 (21 g, 101 mmol) in H₂SO₄ (100 mL) was stirred for 5 min under N₂. Then KNO₃ (20.4 g, 202 mmol) was added portion wise at 0° C. The reaction mixture was stirred at room temperature for 16 h. Then the reaction mixture was diluted with water (300 mL) and stirred for 2 h. The mixture was filtered. The filter cake was wash with water (50 mL×3) and dried to give Compound 84_3 (18 g, 70.4% yield) as a white solid. LCMS: [M+H]⁺=252.0.

### Step 3: General Procedure for Preparation of 3-Bromo-2-(Methoxy-d₃)-5-Nitropyridin-4-Amine (Compound 84_4)

To a solution of Compound 84_3 (18 g, 71 mol) in Methanol-D4 (80 mL) was added K₂CO₃ (29.4 g, 213 mmol). This reaction mixture was stirred at 60° C. for 4 h under N₂ atmosphere. The solvent was removed under reduced pressure. The obtained residue was purified by flash silica gel chromatography to provide the corresponding Compound 84_4 (10.5 g, 59.4% yield) as a white solid. LCMS: [M+H]⁺=251.1.

### Step 4: General procedure for preparation of 3-bromo-4-chloro-2-(methoxy-d₃)-5-nitropyridine (Compound 84_5)

To a solution of Compound 84_4 (5 g, 20 mmol) in MeCN (50 mL) were added isoamyl nitrite (7 g, 60 mmol) and CuCl (6 g, 600 mol). This reaction mixture was stirred at 60° C. for 4 h under N₂ atmosphere. The reaction mixture was diluted with water (20 mL) and extracted with ethyl acetate (20 mL×3). The combined organic phase was washed by brine (30 mL), dried over anhydrous sodium sulfate, filtered, and concentrated in vacuum. The obtained residue was purified by flash silica gel chromatography to provide the corresponding Compound 84_5 (3 g, 59.4% yield) as a white solid. LCMS: [M+H]⁺=270.0.

### Step 5: General procedure for preparation of 6-(((3-bromo-2-(methoxy-d₃)-5-nitropyridin-4-yl)amino) methyl)pyridine-3-sulfonamide (Compound 84_6)

To a solution of Compound 84_5 (2 g, 7.4 mmol) in acetonitrile (20 mL) were added Compound 22_5 (1.38 g, 7.4 mmol) and DIPEA (2.9 g, 22.2 mmol). This reaction mixture was stirred at 60° C. for 4 h. The solvent was removed under reduced pressure. The obtained residue was purified by flash silica gel chromatography to provide the corresponding Compound 84_6 (2.6 g, 83.5% yield) as a yellow solid. LCMS: [M+H]⁺=421.0.

### Step 6: General procedure for preparation of 4-(((2-(methoxy-d₃)-5-nitro-3-phenylpyridin-4-yl)amino) methyl)benzenesulfonamide (Compound 84_7)

To a solution of Compound 84_6 (200 mg, 0.47 mmol) in dioxane/H₂O (10 mL/1 mL) were added phenylboronic acid (86 mg, 0.71 mmol), XPhos-Pd-G2 (37 mg, 0.047 mmol) and K₂CO₃ (195 mg, 1.41 mmol). The mixture was stirred under 100° C. for 16 h under N₂ atmosphere. The solvent was removed under reduced pressure. The obtained residue was purified by flash silica gel chromatography to provide the corresponding Compound 84_7 (160 mg, 80.7% yield) as a yellow solid. LCMS: [M+H]⁺=418.1.

### Step 7: General procedure for preparation of 4-(((5-amino-2-(methoxy-d₃)-3-phenylpyridin-4-yl)amino) methyl)benzenesulfonamide (Compound 84_8)

To a solution of Compound 84_7 (160 mg, 0.38 mmol) in MeOH(3 mL)/DCM (3 mL) was added Pd/C (30 mg, 10% wt). The reaction solution was stirred at room temperature for 16 hr under H₂ atmosphere. The reaction mixture was filtered and the filter cake was washed with MeOH(5 mL×3). The filtrate was concentrated under reduced pressure. The obtained residue was purified by flash silica gel chromatography to provide the corresponding Compound 84_8 (110 mg, 74.1% yield) as a light yellow solid. LCMS: [M+H]⁺=388.1.

### Step 8: General procedure for preparation of 4-((6-(methoxy-d₃)-2-methyl-7-phenyl-1H-imidazo[4,5-c]pyridin-1-yl)methyl)benzenesulfonamide

To a solution of Compound 84_8 (110 mg, 0.28 mmol) in EtOH(5 mL) were added triethyl orthoacetate (461 mg, 2.8 mmol) and Py-HCl (3 mg, 0.028 mmol). The reaction was stirred at 100° C. for 1 h. The solvent was concentrated under reduced pressure. The obtained residue was purified by Prep-HPLC to provide the corresponding example 84 (23 mg, 19.7% yield). LCMS: [M+H]⁺=412.2. ¹H NMR: (400 MHz, DMSO-d₆) δ 8.52 (s, 1H), 7.60 (d, J=8.4 Hz, 2H), 7.34-7.27 (m, 3H), 7.22 (t, J=7.5 Hz, 2H), 7.07 (d, J=7.0 Hz, 2H), 6.66 (d, J=8.3 Hz, 2H), 4.93 (s, 2H), 2.37 (s, 3H).

### Example 85: Synthesis of 5-fluoro-6-((6-(methoxy-d₃)-2-methyl-7-phenyl-1H-imidazo[4,5-c]pyridin-1-yl)methyl)pyridine-3-sulfonamide

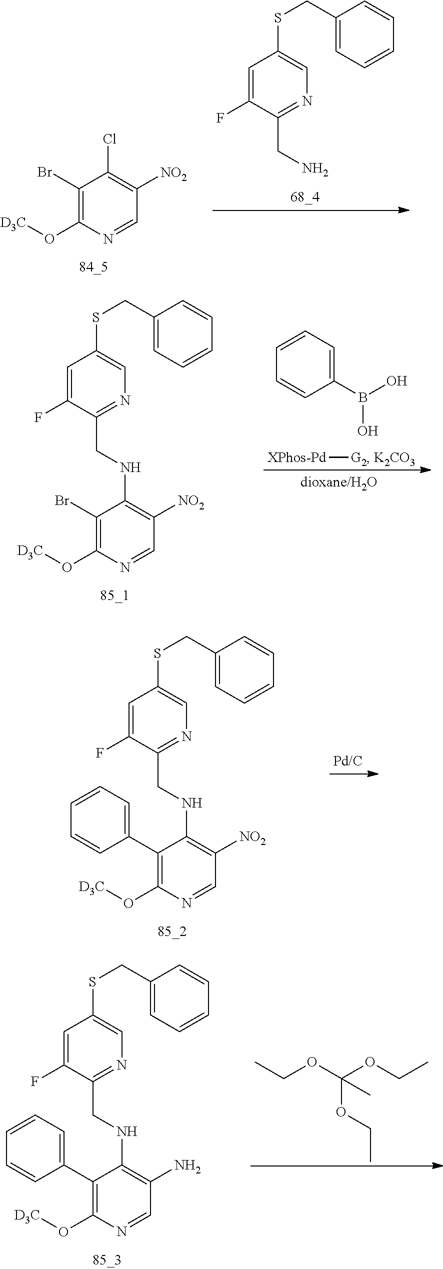

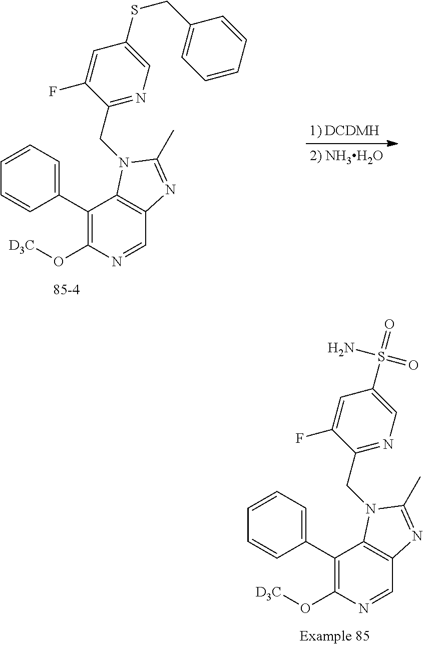 [SMILES: [2H]C([2H])([2H])Oc1ncc2nc(C)n(Cc3ncc(SCc4ccccc4)cc3F)c2c1-c1ccccc1 ; [2H]C([2H])([2H])Oc1ncc2nc(C)n(Cc3ncc(S(N)(=O)=O)cc3F)c2c1-c1ccccc1]

### Step 1: General procedure for preparation of N-((5-(benzylthio)-3-fluoropyridin-2-yl)methyl)-3-bromo-2-(methoxy-d₃)-5-nitropyridin-4-amine (Compound 85_1)

To a solution of Compound 83_5 (1 g, 3.7 mmol) in acetonitrile (10 mL) were added Compound 68_4 (918 mg, 3.7 mmol) and DIPEA (1.5 g, 11.1 mmol). This reaction mixture was stirred at 60° C. for 4 h. The solvent was removed under reduced pressure. The obtained residue was purified by flash silica gel chromatography to provide the corresponding Compound 85_1 (1.3 g, 72.9% yield) as a yellow solid. LCMS: [M+H]⁺=482.0.

### Step 2: General procedure for preparation of N-((5-(benzylthio)-3-fluoropyridin-2-yl)methyl)-2-(methoxy-d₃)-5-nitro-3-phenylpyridin-4-amine (Compound 85_2)

To a solution of Compound 85_1 (500 mg, 1.04 mmol) in dioxane/H₂O (10 mL/1 mL) were added phenylboronic acid (190 mg, 1.55 mmol), XPhos-Pd-G2 (86 mg, 0.11 mmol) and K₂CO₃ (431 mg, 3.12 mmol). The mixture was stirred under 100° C. for 16 h under N₂ atmosphere. The solvent was removed under reduced pressure. The obtained residue was purified by flash silica gel chromatography to provide the corresponding Compound 85_2 (420 mg, 84.5% yield) as a yellow solid. LCMS: [M+H]⁺=480.1.

### Step 3: General procedure for preparation of N₄-((5-(benzylthio)-3-fluoropyridin-2-yl)methyl)-6-(methoxy-d₃)-5-phenylpyridine-3,4-diamine (Compound 85_3)

To a solution of Compound 85_2 (420 mg, 0.88 mmol) in MeOH(8 mL)/DCM (8 mL) was added Pd/C (100 mg, 10% wt). The reaction was stirred at room temperature for 16 h under H₂ atmosphere. The reaction solution was filtered and the filter cake was washed with MeOH(15 mL×3). The filtrate was concentrated under reduced pressure. The obtained residue was purified by flash silica gel chromatography to provide the corresponding Compound 85_3 (320 mg, 81.3% yield) as a light yellow solid. LCMS: [M+H]⁺=450.1.

### Step 4: General procedure for preparation of 1-((5-(benzylthio)-3-fluoropyridin-2-yl)methyl)-6-(methoxy-d₃)-2-methyl-7-phenyl-1H-imidazo[4,5-c]pyridine (Compound 85_4)

To a solution of Compound 85_3 (160 mg, 0.36 mmol) in EtOH(5 mL) were added triethyl orthoacetate (577 mg, 3.56 mmol) and Py-HCl (4 mg, 0.036 mmol). The reaction was stirred at 100° C. for 1 h. The solvent was removed under reduced pressure. The obtained residue was purified by flash silica gel chromatography to provide the corresponding Compound 85_4 (130 mg, 77.1% yield) as a yellow solid. LCMS: [M+H]⁺=474.1.

### Step 5: General procedure for preparation of 5-fluoro-6-((6-(methoxy-d₃)-2-methyl-7-phenyl-1H-imidazo[4,5-c]pyridin-1-yl)methyl)pyridine-3-sulfonamide

To a solution of Compound 85_4 (130 mg, 0.27 mmol) in acetonitrile (5 mL)/AcOH(0.3 mL)/H₂O (0.2 mL) at 0° C. was added drop wise a solution of 1,3-dichloro-5,5-dimethylhydantoin/acetonitrile (133 mg/2 mL, 0.68 mmol). The mixture was stirred under 0° C. for 30 min. The reaction mixture was quenched with water and the aqueous layer was extracted with DCM (10 mL×3). The combined organic layer was concentrated under reduced pressure. The residue was dissolved in acetonitrile (5 mL) and the mixture was cooled to 0° C. Ammonium hydroxide (0.5 mL) was added and the reaction mixture was stirred at 0° C. for 3 min. The reaction mixture was concentrated under reduced pressure. The obtained residue was purified by Prep-HPLC to afford the corresponding example 85 (40 mg, 33.9% yield). LCMS: [M+H]⁺=431.3. ¹H NMR: (400 MHZ, DMSO-d₆) δ 8.49 (s, 1H), 8.47 (s, 1H), 7.77 (dd, J=9.2, 2.0 Hz, 1H), 7.71 (s, 2H), 7.29 (t, J=7.6 Hz, 1H), 7.17 (t, J=7.6 Hz, 2H), 6.85 (d, J=7.6 Hz, 2H), 5.16 (s, 2H), 2.44 (s, 3H).

### Example 86: Synthesis of 6-((2-(difluoromethyl)-6-(methoxy-d₃)-7-phenyl-1H-imidazo[4,5-c]pyridin-1-yl)methyl)-5-fluoropyridine-3-sulfonamide

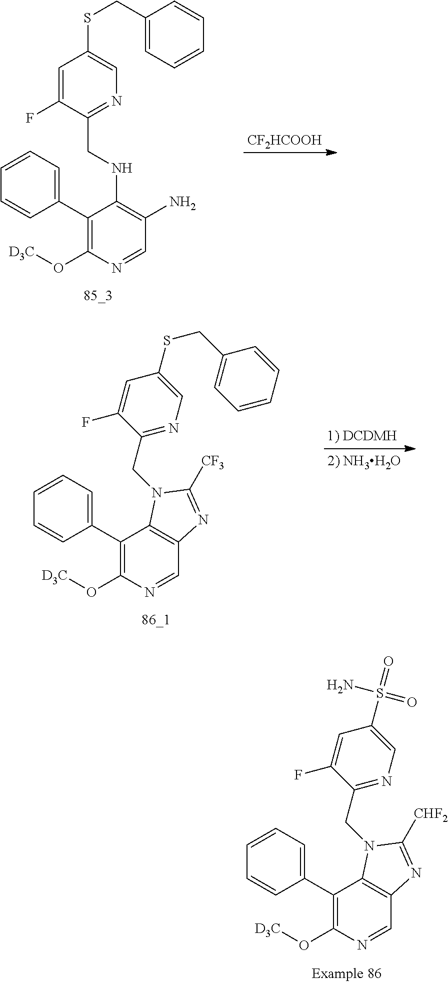

### Step 1: General procedure for preparation of 1-((5-(benzylthio)-3-fluoropyridin-2-yl)methyl)-2-(difluoromethyl)-6-(methoxy-d₃)-7-phenyl-1H-imidazo[4,5-c]pyridine (Compound 86_1)

A solution of Compound 85_3 (160 mg, 0.36 mmol) in trifluoroacetic acid (5 mL) was stirred at 100° C. for 3 h. The solvent was removed under reduced pressure. The obtained residue was purified by flash silica gel chromatography to afford the corresponding Compound 86_1 (80 mg, 44.1% yield) as a yellow solid. LCMS: [M+H]⁺=510.2.

### Step 2: General procedure for preparation of 6-((2-(difluoromethyl)-6-(methoxy-d₃)-7-phenyl-1H-imidazo[4,5-c]pyridin-1-yl)methyl)-5-fluoropyridine-3-sulfonamide

To a solution of Compound 86_1 (80 mg, 0.16 mmol) in acetonitrile (5 mL)/AcOH(0.3 mL)/H₂O (0.2 mL) at 0° C. was added drop wise a solution of 1,3-dichloro-5,5-dimethylhydantoin in acetonitrile (79 mg/2 mL, 0.40 mmol). The mixture was stirred under 0° C. for 30 min. The reaction mixture was quenched with water and the aqueous layer was extracted with DCM (10 mL×3). The combined organic layer was concentrated under reduced pressure. The residue was dissolved in acetonitrile (5 mL) and the mixture was cooled to 0° C. Ammonium hydroxide (0.5 mL) was added and the reaction mixture was stirred at 0° C. for 3 min. The reaction mixture was concentrated under reduced pressure. The obtained residue was purified by Prep-HPLC to afford the corresponding example 86 (17 mg, 23.2% yield). LCMS: [M+H]⁺=467.3. ¹H NMR: (400 MHZ, DMSO-d₆) δ 8.79 (s, 1H), 8.46 (s, 1H), 7.77 (dd, J=9.2, 1.6 Hz, 1H), 7.37 (t, J=51.6 Hz, 1H), 7.29 (t, J=7.6 Hz, 1H), 7.18 (t, J=7.6 Hz, 2H), 7.00 (brs, 2H), 6.91 (d, J=7.2 Hz, 2H), 5.33 (s, 2H).

### Example A: Enzymatic Assay

### ATP-Glo Assay:

1. Diluted compounds in DMSO by hand for 11 points, 3 folds dilution. Then transferred 0.02 μL compounds to 384 assay plate by ECHO.
2. Added 2 μL specified concentration of ENPP1 to 384 assay plate. Centrifuged 1000 RPM for 1 min.
3. Added 2 μL specified concentration of ATP to the assay plate. Centrifuged 1000 RPM for 1 min.
4. Incubated at 25° C. for 60 min.
5. Added 4 μL AMP-Glo Reagent to the assay plates.
6. Centrifuged 1000 RPM for 1 min, incubate at 25° C. for 1 hours.
7. Added 8 μL Kinase Detection Reagent to the assay plates.
8. Centrifuged 1000 RPM for 1 min, incubated at 25° C. for 1 hours. The final assay reaction mixture contained a buffer of 50 mM Tris pH 8.8, 250 mM NaCl and 0.1% BSA
9. Read on Envision for US LUM as RLU.
10. Analyzed the raw data using the equation (V. Data analysis).
   cGAMP-Glo Assay:
1. Diluted compounds in DMSO by hand for 11 points, 3 folds dilution. Then transferred 0.02 μL compounds to 384 assay plate by ECHO.
2. Added 2 μL specified concentration of ENPP1 to 384 assay plate, centrifuged 1000 RPM for 1 min.
3. Added 2 μL specified concentration of 2′3′-cGAMP to the assay plate, centrifuged 1000 RPM for 1 min.
4. Incubated at 25° C. for 60 min.
5. Added 4 μL AMP-Glo Reagent to the assay plates.
6. Centrifuged 1000 RPM for 1 min, incubated at 25° C. for 1 hours.
7. Added 8 μL Kinase Detection Reagent to the assay plates.
8. Centrifuged 1000 RPM for 1 min, incubate at 25° C. for 1 hours. The final assay reaction mixture contained a buffer of 50 mM Tris pH 8.8, 250 mM NaCl and 0.1% BSA
9. Read on Envision for US LUM as RLU.
10. Analyzed the raw data using the equation (V. Data analysis).

The data is shown in Table 2.

TABLE 2

| Ex. | 2′,3′-cGAMP AMP-Glo IC₅₀ (nM) | ATP AMP-Glo IC₅₀ (nM) |
|---|---|---|
| 1 | C | D |
| 2 | D | E |
| 3 | D | E |
| 4 | A | B |
| 5 | B | C |
| 6 | B | B |
| 7 | B | B |
| 8 | C | C |
| 9 | A | A |
| 10 | A | A |
| 11 | D | D |
| 12 | C | C |
| 13 | B | B |
| 14 | A | A |
| 15 | A | A |
| 16 | A | A |
| 17 | B | B |
| 18 | C | D |
| 19 | A | A |
| 20 | A | A |
| 21 | B | C |
| 22 | A | A |
| 23 | A | A |
| 24 | A | A |
| 25 | A | A |
| 26 | A | A |
| 27 | A | A |
| 28 | C | C |
| 29 | A | A |
| 30 | A | A |
| 31 | A | A |
| 32 | A | A |
| 33 | A | A |
| 34 | A | A |
| 35 | B | B |
| 36 | A | A |
| 37 | A | A |
| 38 | A | A |
| 39 | A | A |
| 40 | A | A |
| 41 | A | A |
| 42 | A | A |
| 43 | A | A |
| 44 | A | A |
| 45 | A | A |
| 46 | A | A |
| 47 | A | A |
| 48 | A | A |
| 49 | A | A |
| 50 | A | A |
| 51 | A | A |
| 52 | A | A |
| 53 | A | A |
| 54 | A | A |
| 55 | A | A |
| 56 | A | A |
| 57 | A | A |
| 58 | A | A |
| 59 | A | A |
| 60 | A | A |
| 61 | A | A |
| 62 | A | A |
| 63 | A | A |
| 64 | A | A |
| 65 | A | A |
| 66 | A | A |
| 67 | A | A |
| 68 | A | A |
| 69 | A | A |
| 70 | A | A |
| 71 | A | A |
| 72 | A | A |
| 73 | A | A |
| 74 | A | A |
| 75 | A | A |
| 76 | A | A |
| 77 | A | A |
| 78 | A | A |
| 79 | A | A |
| 80 | A | A |
| 81 | A | A |
| 82 | A | A |
| 84 | A | A |
| 85 | A | A |
| 86 | A | A |

IC₅₀ (nM): 0 < A ≤ 1; 1 < B ≤ 10; 10 < C ≤ 100; 100 < D ≤ 1000; 1000 < E

What is claimed is:

1. A compound of Formula (I), or a pharmaceutically acceptable salt thereof: 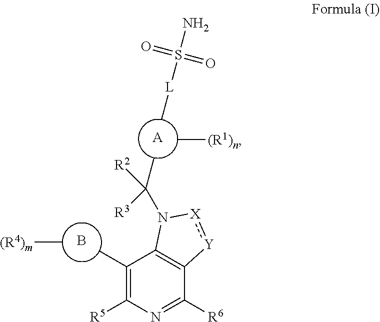
wherein:
Ring A is cycloalkyl, heterocycloalkyl, aryl, or heteroaryl;
L is a bond, —NH—, or —O—;
each R¹ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R¹ on the same atom are taken together to form an oxo;
n is 0-C₆;
R² is hydrogen, halogen, C₁-C₆alkyl, C₁-C₆haloalkyl, C₁-C₆hydroxyalkyl, C₁-C₆aminoalkyl, or C₁-C₆heteroalkyl;
R³ is hydrogen, halogen, C₁-C₆alkyl, C₁-C₆haloalkyl, C₁-C₆ hydroxyalkyl, C₁-C₆aminoalkyl, or C₁-C₆heteroalkyl; [symbol]
is a single bond or a double bond; wherein:
when [symbol] is a double bond, X is N or CR^{X} and Y is N or CR^{Y};
when [symbol] is a single bond, X is C(═O) and Y is NR^{Y1} or C(R^{Y2})₂;
R^{X} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R^{Y} is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
R^{Y1} is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein the alkyl, cycloalkyl, and heterocycloalkyl is optionally substituted with one or more R;
each R^{Y2} is independently hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently and optionally substituted with one or more R;
Ring B is cycloalkyl, heterocycloalkyl, aryl, or heteroaryl;
each R⁴ is independently halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{c}R^{d}(═O) R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R⁴ on the same atom are taken together to form an oxo;
m is 0-4;
R⁵ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R⁶ is hydrogen, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆ alkynyl, cycloalkyl, heterocycloalkyl, aryl, or heteroaryl;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{a} are taken together with the atom to which they are attached to form a heterocycloalkyl optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or two R^{b} are taken together with the atom to which they are attached to form a heterocycloalkyl optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein the alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl;
or two R on the same atom form an oxo.

2. The compound of claim 1, or a pharmaceutically acceptable salt thereof, wherein each R is independently halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆hydroxyalkyl, C₁-C₆aminoalkyl, or C₁-C₆heteroalkyl; or two R on the same atom form an oxo.

3. The compound of claim 1 or 2, or a pharmaceutically acceptable salt thereof, wherein the compound is of Formula (Ia):

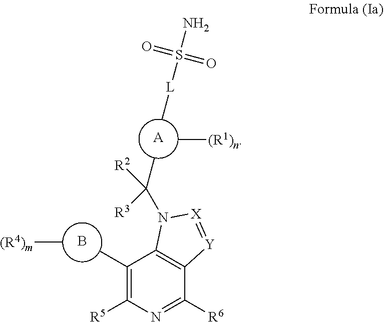

4. The compound of any one of claims 1 to 3, or a pharmaceutically acceptable salt thereof, wherein X is N and Y is N.

5. The compound of any one of claims 1 to 3, or a pharmaceutically acceptable salt thereof, wherein X is CR^{X} and Y is N.

6. The compound of any one of claims 1 to 3, or a pharmaceutically acceptable salt thereof, wherein X is N and Y is CR^{Y}.

7. The compound of any one of claims 1 to 3, or a pharmaceutically acceptable salt thereof, wherein X is CR^{X} and Y is CR^{Y}.

8. The compound of any one of claims 1-7, or a pharmaceutically acceptable salt thereof, wherein R^{X} is hydrogen, halogen, C₁-C₆ alkyl, C₁-C₆haloalkyl, or cycloalkyl.

9. The compound of any one of claims 1-8, or a pharmaceutically acceptable salt thereof, wherein R^{X} is hydrogen, C₁-C₆ alkyl, C₁-C₆haloalkyl, or cycloalkyl.

10. The compound of any one of claims 1-9, or a pharmaceutically acceptable salt thereof, wherein R^{Y} is hydrogen, halogen, C₁-C₆ alkyl, C₁-C₆haloalkyl, or cycloalkyl.

11. The compound of any one of claims 1-10, or a pharmaceutically acceptable salt thereof, wherein R^{Y} is hydrogen or C₁-C₆alkyl.

12. The compound of claim 1 or 2, or a pharmaceutically acceptable salt thereof, wherein the compound is of Formula (Ib):

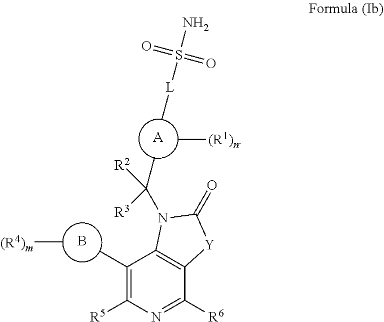

13. The compound of claim 12, or a pharmaceutically acceptable salt thereof, wherein Y is NR^{Y1}.

14. The compound of claim 12, or a pharmaceutically acceptable salt thereof, wherein Y is C(R^{Y2})₂.

15. The compound of any one of claim 1, 2, 12, or 13, or a pharmaceutically acceptable salt thereof, wherein R^{Y1} is hydrogen, C₁-C₆alkyl, or C₁-C₆haloalkyl.

16. The compound of any one of claim 1, 2, 12, or 14, or a pharmaceutically acceptable salt thereof, wherein each R^{Y2} is independently hydrogen, halogen, C₁-C₆alkyl, or C₁-C₆haloalkyl.

17. The compound of any one of claims 1-16, or a pharmaceutically acceptable salt thereof, wherein Ring A is cycloalkyl or heterocycloalkyl.

18. The compound of any one of claims 1-17, or a pharmaceutically acceptable salt thereof, wherein Ring A is cycloalkyl.

19. The compound of any one of claims 1-17, or a pharmaceutically acceptable salt thereof, wherein Ring A is heterocycloalkyl.

20. The compound of any one of claims 1-16, or a pharmaceutically acceptable salt thereof, wherein Ring A is aryl or heteroaryl.

21. The compound of any one of claim 1-16 or 20, or a pharmaceutically acceptable salt thereof, wherein Ring A is aryl.

22. The compound of any one of claim 1-16 or 20, or a pharmaceutically acceptable salt thereof, wherein Ring A is heteroaryl.

23. The compound of any one of claims 1-16, or a pharmaceutically acceptable salt thereof, wherein Ring A is phenyl or pyridinyl.

24. The compound of any one of claims 1-23, or a pharmaceutically acceptable salt thereof, wherein L is a bond.

25. The compound of any one of claims 1-23, or a pharmaceutically acceptable salt thereof, wherein L is —NH—.

26. The compound of any one of claims 1-23, or a pharmaceutically acceptable salt thereof, wherein L is —O—.

27. The compound of any one of claims 1-26, or a pharmaceutically acceptable salt thereof, wherein each R¹ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

28. The compound of any one of claims 1-27, or a pharmaceutically acceptable salt thereof, wherein each R¹ is independently halogen, —CN, —OH, —OR^{a}, —NR^{c}R^{d}, C₁-C₆alkyl, or C₁-C₆haloalkyl.

29. The compound of any one of claims 1-28, or a pharmaceutically acceptable salt thereof, wherein each R¹ is independently halogen, C₁-C₆alkyl, or C₁-C₆haloalkyl.

30. The compound of any one of claims 1-29, or a pharmaceutically acceptable salt thereof, wherein n is 0-2.

31. The compound of any one of claims 1-30, or a pharmaceutically acceptable salt thereof, wherein n is 0 or 1.

32. The compound of any one of claims 1-30, or a pharmaceutically acceptable salt thereof, wherein n is 1 or 2.

33. The compound of any one of claims 1-32, or a pharmaceutically acceptable salt thereof, wherein R² is hydrogen, halogen, C₁-C₆alkyl, or C₁-C₆haloalkyl.

34. The compound of any one of claims 1-33, or a pharmaceutically acceptable salt thereof, wherein R² is hydrogen or C₁-C₆alkyl.

35. The compound of any one of claims 1-34, or a pharmaceutically acceptable salt thereof, wherein R² is hydrogen.

36. The compound of any one of claims 1-35, or a pharmaceutically acceptable salt thereof, wherein R³ is hydrogen, halogen, C₁-C₆alkyl, or C₁-C₆haloalkyl.

37. The compound of any one of claims 1-36, or a pharmaceutically acceptable salt thereof, wherein R³ is hydrogen or C₁-C₆alkyl.

38. The compound of any one of claims 1-37, or a pharmaceutically acceptable salt thereof, wherein R³ is hydrogen.

39. The compound of any one of claims 1-38, or a pharmaceutically acceptable salt thereof, wherein Ring B is cycloalkyl or heterocycloalkyl.

40. The compound of any one of claims 1-39, or a pharmaceutically acceptable salt thereof, wherein Ring B is cycloalkyl.

41. The compound of any one of claims 1-39, or a pharmaceutically acceptable salt thereof, wherein Ring B is heterocycloalkyl.

42. The compound of any one of claims 1-38, or a pharmaceutically acceptable salt thereof, wherein Ring B is aryl or heteroaryl.

43. The compound of any one of claim 1-38 or 42, or a pharmaceutically acceptable salt thereof, wherein Ring B is aryl.

44. The compound of claim 43, or a pharmaceutically acceptable salt thereof, wherein Ring B is phenyl.

45. The compound of any one of claim 1-38 or 42, or a pharmaceutically acceptable salt thereof, wherein Ring B is heteroaryl.

46. The compound of claim 45, or a pharmaceutically acceptable salt thereof, wherein Ring B is 5- or 6-membered heteroaryl containing 1-3 nitrogen and 0-1 oxygen.

47. The compound of any one of claims 1-46, or a pharmaceutically acceptable salt thereof, wherein each R⁴ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, or C₁-C₆haloalkyl.

48. The compound of any one of claims 1-47, or a pharmaceutically acceptable salt thereof, wherein each R⁴ is independently halogen, —CN, —OH, —OR^{a}, —NR^{c}R^{d}, C₁-C₆alkyl, or C₁-C₆haloalkyl.

49. The compound of any one of claims 1-48, or a pharmaceutically acceptable salt thereof, wherein each R⁴ is independently halogen, C₁-C₆alkyl, or C₁-C₆haloalkyl.

50. The compound of any one of claims 1-49, or a pharmaceutically acceptable salt thereof, wherein each R⁴ is independently C₁-C₆alkyl.

51. The compound of any one of claims 1-50, or a pharmaceutically acceptable salt thereof, wherein m is 0-2.

52. The compound of any one of claims 1-51, or a pharmaceutically acceptable salt thereof, wherein m is 0 or 1.

53. The compound of any one of claims 1-52, or a pharmaceutically acceptable salt thereof, wherein R⁵ is hydrogen, halogen, —CN, —OH, —OR^{a}, —NR^{c}R^{d}, C₁-C₆ alkyl, or C₁-C₆haloalkyl.

54. The compound of any one of claims 1-53, or a pharmaceutically acceptable salt thereof, wherein R⁵ is hydrogen, —CN, —OR^{a}, C₁-C₆alkyl, or C₁-C₆haloalkyl.

55. The compound of any one of claims 1-54, or a pharmaceutically acceptable salt thereof, wherein R⁵ is —OR^{a} or C₁-C₆alkyl.

56. The compound of any one of claims 1-55, or a pharmaceutically acceptable salt thereof, wherein R⁵ is C₁-C₆alkoxyl or C₁-C₆alkyl.

57. The compound of any one of claims 1-56, or a pharmaceutically acceptable salt thereof, wherein R⁶ is hydrogen, halogen, —CN, —OH, —OR^{a}, —NR^{c}R^{d}, C₁-C₆alkyl, or C₁-C₆haloalkyl.

58. The compound of any one of claims 1-57, or a pharmaceutically acceptable salt thereof, wherein R⁶ is hydrogen, halogen, C₁-C₆alkyl, or C₁-C₆haloalkyl.

59. The compound of any one of claims 1-58, or a pharmaceutically acceptable salt thereof, wherein R⁶ is hydrogen or C₁-C₆alkyl.

60. The compound of any one of claims 1-59, or a pharmaceutically acceptable salt thereof, wherein one or more of R, R¹, R², R³, R⁴, R⁵, R⁶, R^{X}, R^{Y}, R^{Y1}, R^{Y2}, R^{a}, R^{b}, Re, and R^{d} groups comprise deuterium at a percentage higher than the natural abundance of deuterium.

61. A compound selected from table 1, or a pharmaceutically acceptable salt thereof.

62. A pharmaceutical composition comprising a therapeutically effective amount of a compound of any one of claims 1-61, or a pharmaceutically acceptable salt thereof, and a pharmaceutically acceptable excipient.

63. A method of treating cancer in a subject, the method comprising administering to the subject a compound of any one of claims 1-61, or a pharmaceutically acceptable salt thereof, or a pharmaceutical composition of claim 62.

64. The method of claim 63, wherein the cancer is a solid tumor.

65. The method of claim 64, wherein the solid tumor is breast cancer, lung cancer, ovarian cancer, head and neck cancer, melanoma, pancreatic cancer, liver cancer, gastric cancer, colorectal cancer, or sarcoma.

66. The method of claim 64, wherein the cancer is a hematologic malignancy.

67. The method of claim 66, wherein the hematologic malignancy is a leukemia, a lymphoma, or a myeloma.

68. The method of claim 66, wherein the hematologic malignancy is a B-cell malignancy.

69. The method of claim 66, wherein the hematologic malignancy is multiple myeloma.

70. The method of any one of the claims 63-69, wherein the cancer is a relapsed or refractory cancer.

71. The method of any one of the claims 63-70, wherein the cancer is a metastatic cancer.

72. A method of treating an infection in a subject in need thereof comprising administering a compound of any one of claims 1-61, or a pharmaceutically acceptable salt thereof, or a pharmaceutical composition of claim 62.

73. The method of claim 72, wherein the infection is a viral infection.

74. The method of claim 73, wherein the viral infection is due to a DNA virus.

75. The method of claim 73 or 74, wherein the viral infection is due to a herpesvirus.

76. The method of claim 75, wherein the herpesvirus is selected from herpes simplex viruses 1 (HSV-1), herpes simplex viruses 2 (HSV-2), varicella-zoster virus (VZV), Epstein-Barr virus (EBV), human cytomegalovirus (HCMV), human herpesvirus 6A (HHV-6A), human herpesvirus 6B (HHV-6B), human herpesvirus 7 (HHV-7), and Kaposi's sarcoma-associated herpesvirus (KSHV).

77. The method of claim 75 or 76, wherein the herpesvirus is herpes simplex viruses 1 (HSV-1).

78. The method of claim 73 or 74, wherein the viral infection is due to a retrovirus.

79. The method of claim 78, wherein the retrovirus is human immunodeficiency virus (HIV).

80. The method of claim 73 or 74, wherein the viral infection is due to a hepatitis virus.

81. The method of claim 80, wherein the hepatitis virus is hepatitis B virus (HBV) or hepatitis D virus (HDV).

82. The method of claim 73 or 74, wherein the viral infection is due to vaccinia virus (VACV), adenovirus, or human papillomaviruses (HPV).

83. The method of claim 82, wherein the viral infection is due to a RNA virus.

84. The method of claim 73 or 74, wherein the viral infection is due to dengue fever virus, yellow fever virus, Ebola virus, Marburg virus, Venezuelan encephalitis virus, or zika virus.
